SEQUENCE LISTING (121 sequences)

SEQ ID NO: 1 - protein, 257 aa, Homo sapiens
MAQRMTTQLLLLLVWVAVVGEAQTRIAWARTELLNVCMNAKHHKEKPGPEDKLHEQCRPWRKNACCSTNTSQEAHKDVSYLYRFNWNHCGEMAPACKRHFIQDTCLYECSPNLGPWIQQVDQSWRKERVLNVPLCKEDCEQWWEDCRTSYTCKSNWHKGWNWTSGFNKCAVGAACQPFHFYFPTPTVLCNEIWTHSYKVSNYSRGSGRCIQMWFDPAQGNPNEEVARFYAAAMSGAGPWAAWPFLLSLALMLLWLLS

SEQ ID NO: 2 - protein, 114 aa, synthetic construct
QVQLQESGGGLVQAGGSLRLSCAASGSGFSGMIMGWYRQVPGKQRELVATVTSHGTTNYADSVKGRFTISRDNAKNTVLLQMNSLKPEDTAVYYCNAHFPGIYWGKGTQVTVSS

SEQ ID NO: 3 - protein, 10 aa, synthetic construct
GSGFSGMIMG

SEQ ID NO: 4 - protein, 16 aa, synthetic construct
TVTSHGTTNYADSVKG

SEQ ID NO: 5 - protein, 8 aa, synthetic construct
NAHFPGIY

SEQ ID NO: 6 - protein, 114 aa, synthetic construct
QVQLQESGGGLVQAGGSLRLSCAASGSGFSGMIMGWYRQAPGKQRELVATITSGGTTNYADSVKGRFTISRDNAKNTVLLQMNSLKPEDTAVYYCNAHFPGIYWGKGTQVTVSS

SEQ ID NO: 7 - protein, 16 aa, synthetic construct
TITSGGTTNYADSVKG

SEQ ID NO: 8 - protein, 127 aa, synthetic construct
QVQLQESGGGLVQPGGSLRVSCAASGFPFSTVYMSWVRQAPGKGLEWVSGINNDGGVIDYADSVKGRFTISRDNAKNTLYLQLNSLKPEDTALYFCARGRSFVATLSSSLRGQGTQVTVSSHHHHHH

SEQ ID NO: 9 - protein, 10 aa, synthetic construct
GFPFSTVYMS

SEQ ID NO: 10 - protein, 17 aa, synthetic construct
GINNDGGVIDYADSVKG

SEQ ID NO: 11 - protein, 15 aa, synthetic construct
ARGRSFVATLSSSLR

SEQ ID NO: 12 - protein, 121 aa, synthetic construct
QVQLQESGGGLVQAGGSLRVSCAASGFPFSTVYMSWVRQAPGKGLEWVSGINSNGEIIDYADSVKGRFTISRDNAKNTLYLQLNSLKPEDTALYFCARGRAFVATLPSSLRGQGTQVTVSS

SEQ ID NO: 13 - protein, 17 aa, synthetic construct
GINSNGEIIDYADSVKG

SEQ ID NO: 14 - protein, 15 aa, synthetic construct
ARGRAFVATLPSSLR

SEQ ID NO: 15 - protein, 121 aa, synthetic construct
QVQLQESGGGLVQPGGSLRVSCAASGFPFSTYYMSWVRRAPGKGLEWVSGINSDGGVIDYADSVKGRFTISRDNAKNTLYLQLSSLKPEDTALYFCARGRAFVATLPSSLRGQGTQVTVSS

SEQ ID NO: 16 - protein, 10 aa, synthetic construct
GFPFSTYYMS

SEQ ID NO: 17 - protein, 17 aa, synthetic construct
GINSDGGVIDYADSVKG

SEQ ID NO: 18 - protein, 121 aa, synthetic construct
QVQLQESGGGLVQAGGSLRLSCAASGFPFSTYYMSWVRQAPGKGLEWVSGINSNGGVIDYADSVKGRFTISRDNAKNTLYLQLNSLKPEDTALYFCARGRAFVATLPSSLRGQGTQVTVSS

SEQ ID NO: 19 - protein, 17 aa, synthetic construct
GINSNGGVIDYADSVKG

SEQ ID NO: 20 - protein, 121 aa, synthetic construct
QVQLQESGGGLVQPGGSLRITCVASGFPFSTYYMSWVRQAPGKGLEWVSGINSNGGVIDYADSVKGRFTISRDNAKNTLYLQLNSLKPEDTALYFCARGRAFVATMPSSLRGQGTQVTVSS

SEQ ID NO: 21 - protein, 15 aa, synthetic construct
ARGRAFVATMPSSLR

SEQ ID NO: 22 - protein, 121 aa, synthetic construct
QVQLQESGGGLVQPGGSLRLSCAASGFPFSTYYMSWVRQAPGKGLEWVSGINSNGGVIDYADSVKGRFTISRDNAKNTLYLQLNSLKPEDTALYFCARGRAFVATMPSSLRGQGTQVTVSS

SEQ ID NO: 23 - protein, 121 aa, synthetic construct
QVQLQESGGGLVQPGGSLRLSCAASGSTLDRYEIGWFRQAPGKGRERVSCISTTSSTYEDSVKGRFTISRDNAKNTVYLQMNSLKPEDTAVYYCATVRVAWSTCFPTGDSWGKGTQVTVSS

SEQ ID NO: 24 - protein, 10 aa, synthetic construct
GSTLDRYEIG

SEQ ID NO: 25 - protein, 20 aa, synthetic construct
CISTTSSTYEDSVKGRFTIS

SEQ ID NO: 26 - protein, 16 aa, synthetic construct
ATVRVAWSTCFPTGDS

SEQ ID NO: 27 - protein, 117 aa, synthetic construct
QVQLQESGGGLVQAGGSLRVSCAASGIIFSMNGMGWFRQAPGKQRELVASITSGGSTKYADSVKGRFTISRDNAKNTVYLQMNSLKPEDTAVYYCNIQFGSRWSTYWGQGTQVTVSS

SEQ ID NO: 28 - protein, 10 aa, synthetic construct
GIIFSMNGMG

SEQ ID NO: 29 - protein, 16 aa, synthetic construct
SITSGGSTKYADSVKG

SEQ ID NO: 30 - protein, 12 aa, synthetic construct
NIQFGSRWSTYW

SEQ ID NO: 31 - protein, 16 aa, synthetic construct
  VARIANT 2: note = V or I
TXTSHGTTNYADSVKG

SEQ ID NO: 32 - protein, 16 aa, synthetic construct
  VARIANT 2: note = V or I
  VARIANT 5: note = H or G
TXTSXGTTNYADSVKG

SEQ ID NO: 33 - protein, 10 aa, synthetic construct
  VARIANT 7: note = V or Y
GFPFSTXYMS

SEQ ID NO: 34 - protein, 17 aa, synthetic construct
  VARIANT 4: note = N or S
  VARIANT 5: note = D or N
  VARIANT 7: note = G or E
  VARIANT 8: note = V or I
GINXXGXXIDYADSVKG

SEQ ID NO: 35 - protein, 15 aa, synthetic construct
  VARIANT 5: note = S or A
  VARIANT 10: note = L or M
  VARIANT 11: note = S or P
ARGRXFVATXXSSLR

SEQ ID NO: 36 - protein, 114 aa, synthetic construct
QVQLQESGGGLVQAGGSLRLSCAASGSGFSGMIMGWYRQAPGKQRELVATVTSHGTTNYADSVKGRFTISREDSKNTVLLQMNSLKPEDTAVYYCNAHFPGIYWGKGTQVTVSS

SEQ ID NO: 37 - protein, 123 aa, synthetic construct
EVQLVESGGGLVQPGGSLRLSCAASGSGFSGMIMGWYRQAPGKQRELVATVTSHGTTNYADSVKGRFTISRDNAKNTVYLQMNSLRPEDTAVYYCNAHFPGIYWGKGTLVTVSSAAAHHHHHH

SEQ ID NO: 38 - protein, 123 aa, synthetic construct
EVQLVESGGGLVQPGGSLRLSCAASGSGFSGMIMGWYRQAPGKQRELVATVTSHGTTNYADSVKGRFTISRDNAKNTVYLQMNSLRPEDTAVYYCNAHFPGIYWGQGTLVTVSSAAAHHHHHH

SEQ ID NO: 39 - protein, 130 aa, synthetic construct
EVQLVESGGGLVQPGGSLRVSCAASGFPFSTVYMSWVRQAPGKGLEWVSGINNDGGVIDYADSVKGRFTISRDNAKNTLYLQLNSLKPEDTALYFCARGRSFVATLSSSLRGQGTLVTVSSAAAHHHHHH

SEQ ID NO: 40 - protein, 123 aa, synthetic construct
EVQLVESGGGLVQAGGSLRLSCAASGSGFSGMIMGWYRQVPGKQRELVATVTSHGTTNYADSVKGRFTISRDNAKNTVLLQMNSLKPEDTAVYYCNAHFPGIYWGKGTLVTVSSAAAHHHHHH

SEQ ID NO: 41 - protein, 123 aa, synthetic construct
EVQLVESGGGLVQAGGSLRLSCAASGSGFSGMIMGWYRQAPGKQRELVATVTSHGTTNYADSVKGRFTISREDSKNTVLLQMNSLKPEDTAVYYCNAHFPGIYWGKGTLVTVSSAAAHHHHHH

SEQ ID NO: 42 - protein, 148 aa, synthetic construct
EVQLVESGGGLVQPGGSLRLSCAASGSGFSGMIMGWYRQAPGKQRELVATVTSHGTTNYADSVKGRFTISRDNAKNTVYLQMNSLRPEDTAVYYCNAHFPGIYWGKGTLVTVSSAAADYKDHDGDYKDHDIDYKDDDDKGAAHHHHHH

SEQ ID NO: 43 - protein, 148 aa, synthetic construct
EVQLVESGGGLVQPGGSLRLSCAASGSGFSGMIMGWYRQAPGKQRELVATVTSHGTTNYADSVKGRFTISRDNAKNTVYLQMNSLRPEDTAVYYCNAHFPGIYWGQGTLVTVSSAAADYKDHDGDYKDHDIDYKDDDDKGAAHHHHHH

SEQ ID NO: 44 - protein, 148 aa, synthetic construct
EVQLVESGGGLVQAGGSLRLSCAASGSGFSGMIMGWYRQAPGKQRELVATVTSHGTTNYADSVKGRFTISREDSKNTVLLQMNSLKPEDTAVYYCNAHFPGIYWGKGTLVTVSSAAADYKDHDGDYKDHDIDYKDDDDKGAAHHHHHH

SEQ ID NO: 45 - protein, 123 aa, synthetic construct
EVQLVESGGGLVQPGGSLRLSCAASESPFSGMIMGWYRQAPGKQRELVATVTSHGTTNYADSVKGRFTISREGAKNTVYLQMNSLRPEDTAVYYCNAHFPGIYWGKGTLVTVSSAAAHHHHHH

SEQ ID NO: 46 - protein, 159 aa, synthetic construct
EVQLVESGGGLVQPGGSLRLSCAASGSGFSGMIMGWYRQAPGKQRELVATVTSHGTTNYADSVKGRFTISRDNAKNTVYLQMNSLRPEDTAVYYCNAHFPGIYWGKGTLVTVSSAAADYKDHDGDYKDHDIDYKDDDDKGAAHHHHHHGGGGSRRPYIL

SEQ ID NO: 47 - protein, 148 aa, synthetic construct
EVQLVESGGGLVQPGGSLRLSCAASGSGFSGMIMGWYRQAPGKQRELVATVTSHGTTNYADSVKGRFTISRPGAKNTVYLQMNSLRPEDTAVYYCNAHFPGIYWGKGTLVTVSSAAADYKDHDGDYKDHDIDYKDDDDKGAAHHHHHH

SEQ ID NO: 48 - protein, 148 aa, synthetic construct
EVQLVESGGGLVQPGGSLRLSCAASGSTFIGMIMGWYRQAPGKQRELVATVTSHGTTNYADSVKGRFTISRDNAKNTVYLQMNSLRPEDTAVYYCNAHFPGIYWGKGTLVTVSSAAADYKDHDGDYKDHDIDYKDDDDKGAAHHHHHH

SEQ ID NO: 49 - protein, 148 aa, synthetic construct
EVQLVESGGGLVQPGGSLRLSCAASGSTFSGMLMGWYRQAPGKQRELVATVTSHGTTNYADSVKGRFTISRENAKNTVYLQMNSLRPEDTAVYYCNAHFPGIYWGKGTLVTVSSAAADYKDHDGDYKDHDIDYKDDDDKGAAHHHHHH

SEQ ID NO: 50 - protein, 148 aa, synthetic construct
EVQLVESGGGLVQPGGSLRLSCAASGSGFIGMIMGWYRQAPGKQRELVATVTSHGTTNYADSVKGRFTISREGAKNTVYLQMNSLRPEDTAVYYCNAHFPGIYWGKGTLVTVSSAAADYKDHDGDYKDHDIDYKDDDDKGAAHHHHHH

SEQ ID NO: 51 - protein, 148 aa, synthetic construct
EVQLVESGGGLVQPGGSLRLSCAASESGFSGMLMGWYRQAPGKQRELVATVTSHGTTNYADSVKGRFTISRDNAKNTVYLQMNSLRPEDTAVYYCNAHFPGIYWGKGTLVTVSSAAADYKDHDGDYKDHDIDYKDDDDKGAAHHHHHH

SEQ ID NO: 52 - protein, 148 aa, synthetic construct
EVQLVESGGGLVQPGGSLRLSCAASGSGFSGMIMGWYRQAPGKQRELVATVTSHGTTNYADSVKGRFTISRDPAKNTVYLQMNSLRPEDTAVYYCNAHFPGIYWGKGTLVTVSSAAADYKDHDGDYKDHDIDYKDDDDKGAAHHHHHH

SEQ ID NO: 53 - protein, 148 aa, synthetic construct
EVQLVESGGGLVQPGGSLRLSCAASESTFIGMLMGWYRQAPGKQRELVATVTSHGTTNYADSVKGRFTISRDNAKNTVYLQMNSLRPEDTAVYYCNAHFPGIYWGKGTLVTVSSAAADYKDHDGDYKDHDIDYKDDDDKGAAHHHHHH

SEQ ID NO: 54 - protein, 148 aa, synthetic construct
EVQLVESGGGLVQPGGSLRLSCAASGSGFIGMIMGWYRQAPGKQRELVATVTSHGTTNYADSVKGRFTISRPNAKNTVYLQMNSLRPEDTAVYYCNAHFPGIYWGKGTLVTVSSAAADYKDHDGDYKDHDIDYKDDDDKGAAHHHHHH

SEQ ID NO: 55 - protein, 148 aa, synthetic construct
EVQLVESGGGLVQPGGSLRLSCAASESGFSGMIMGWYRQAPGKQRELVATVTSHGTTNYADSVKGRFTISRDNAKNTVYLQMNSLRPEDTAVYYCNAHFPGIYWGKGTLVTVSSAAADYKDHDGDYKDHDIDYKDDDDKGAAHHHHHH

SEQ ID NO: 56 - protein, 148 aa, synthetic construct
EVQLVESGGGLVQPGGSLRLSCAASGSTFSGMIMGWYRQAPGKQRELVATVTSHGTTNYADSVKGRFTISRPNAKNTVYLQMNSLRPEDTAVYYCNAHFPGIYWGKGTLVTVSSAAADYKDHDGDYKDHDIDYKDDDDKGAAHHHHHH

SEQ ID NO: 57 - protein, 148 aa, synthetic construct
EVQLVESGGGLVQPGGSLRLSCAASGSGFSGMIMGWYRQAPGKQRELVATVTSHGTTNYADSVKGRFTISREGAKNTVYLQMNSLRPEDTAVYYCNAHFPGIYWGKGTLVTVSSAAADYKDHDGDYKDHDIDYKDDDDKGAAHHHHHH

SEQ ID NO: 58 - protein, 148 aa, synthetic construct
EVQLVESGGGLVQPGGSLRLSCAASGSGFSGMIMGWYRQAPGKQRELVATRTSHGTTNYADSVKGRFTISRENAKNTVYLQMNSLRPEDTAVYYCNAHFPGIYWGKGTLVTVSSAAADYKDHDGDYKDHDIDYKDDDDKGAAHHHHHH

SEQ ID NO: 59 - protein, 148 aa, synthetic construct
EVQLVESGGGLVQPGGSLRLSCAASGSGFIGMLMGWYRQAPGKQRELVATVTSHGTTNYADSVKGRFTISRDPAKNTVYLQMNSLRPEDTAVYYCNAHFPGIYWGKGTLVTVSSAAADYKDHDGDYKDHDIDYKDDDDKGAAHHHHHH

SEQ ID NO: 60 - protein, 148 aa, synthetic construct
EVQLVESGGGLVQPGGSLRLSCAASGSGFSGMLMGWYRQAPGKQRELVATVTSHGTTNYADSVKGRFTISRDPAKNTVYLQMNSLRPEDTAVYYCNAHFPGIYWGKGTLVTVSSAAADYKDHDGDYKDHDIDYKDDDDKGAAHHHHHH

SEQ ID NO: 61 - protein, 148 aa, synthetic construct
EVQLVESGGGLVQPGGSLRLSCAASGSGFSGMIMGWYRQAPGKQRELVATRTSHGTTNYADSVKGRFTISREGAKNTVYLQMNSLRPEDTAVYYCNAHFPGIYWGKGTLVTVSSAAADYKDHDGDYKDHDIDYKDDDDKGAAHHHHHH

SEQ ID NO: 62 - protein, 148 aa, synthetic construct
EVQLVESGGGLVQPGGSLRLSCAASGSGFSGMLMGWYRQAPGKQRELVATVTSHGTTNYADSVKGRFTISRPGAKNTVYLQMNSLRPEDTAVYYCNAHFPGIYWGKGTLVTVSSAAADYKDHDGDYKDHDIDYKDDDDKGAAHHHHHH

SEQ ID NO: 63 - protein, 148 aa, synthetic construct
EVQLVESGGGLVQPGGSLRLSCAASESPFSGMIMGWYRQAPGKQRELVATVTSHGTTNYADSVKGRFTISREGAKNTVYLQMNSLRPEDTAVYYCNAHFPGIYWGKGTLVTVSSAAADYKDHDGDYKDHDIDYKDDDDKGAAHHHHHH

SEQ ID NO: 64 - protein, 148 aa, synthetic construct
EVQLVESGGGLVQPGGSLRLSCAASESPFSGMIMGWYRQAPGKQRELVATVTSHGTTNYADSVKGRFTISRDNAKNTVYLQMNSLRPEDTAVYYCNAHFPGIYWGKGTLVTVSSAAADYKDHDGDYKDHDIDYKDDDDKGAAHHHHHH

SEQ ID NO: 65 - protein, 148 aa, synthetic construct
EVQLVESGGGLVQPGGSLRLSCAASESGFSGMIMGWYRQAPGKQRELVATVTSHGTTNYADSVKGRFTISRENAKNTVYLQMNSLRPEDTAVYYCNAHFPGIYWGKGTLVTVSSAAADYKDHDGDYKDHDIDYKDDDDKGAAHHHHHH

SEQ ID NO: 66 - protein, 255 aa, Homo sapiens
MVWKWMPLLLLLVCVATMCSAQDRTDLLNVCMDAKHHKTKPGPEDKLHDQCSPWKKNACCTASTSQELHKDTSRLYNFNWDHCGKMEPACKRHFIQDTCLYECSPNLGPWIQQVNQSWRKERFLDVPLCKEDCQRWWEDCHTSHTCKSNWHRGWDWTSGVNKCPAGALCRTFESYFPTPAALCEGLWSHSYKVSNYSRGSGRCIQMWFDSAQGNPNEEVARFYAAAMHVNAGEMLHGTGGLLLSLALMLQLWLLG

SEQ ID NO: 67 - protein, 257 aa, synthetic construct
MAQRMTTQLLLLLVWVAVVGEAQTRIAWARTDLLNVCMDAKHHKTKPGPEDKLHDQCSPWKKNACCSTNTSQEAHKDVSYLYRFNWNHCGEMAPACKRHFIQDTCLYECSPNLGPWIQQVDQSWRKERVLNVPLCKEDCEQWWEDCRTSYTCKSNWHKGWNWTSGFNKCAVGAACQPFHFYFPTPTVLCNEIWTHSYKVSNYSRGSGRCIQMWFDSAQGNPNEEVARFYAAAMSGAGPWAAWPFLLSLALMLLWLLS

SEQ ID NO: 68 - protein, 257 aa, synthetic construct
MAQRMTTQLLLLLVWVAVVGEAQTRIAWARTELLNVCMNAKHHKEKPGPEDKLHEQCRPWRKNACCTASTSQELHKDTSRLYNFNWDHCGKMEPACKRHFIQDTCLYECSPNLGPWIQQVDQSWRKERVLNVPLCKEDCEQWWEDCRTSYTCKSNWHKGWNWTSGFNKCAVGAACQPFHFYFPTPTVLCNEIWTHSYKVSNYSRGSGRCIQMWFDPAQGNPNEEVARFYAAAMSGAGPWAAWPFLLSLALMLLWLLS

SEQ ID NO: 69 - protein, 257 aa, synthetic construct
MAQRMTTQLLLLLVWVAVVGEAQTRIAWARTELLNVCMNAKHHKEKPGPEDKLHEQCRPWRKNACCSTNTSQEAHKDVSYLYRFNWNHCGEMAPACKRHFIQDTCLYECSPNLGPWIQQVNQSWRKERFLDVPLCKEDCEQWWEDCRTSYTCKSNWHKGWNWTSGFNKCAVGAACQPFHFYFPTPTVLCNEIWTHSYKVSNYSRGSGRCIQMWFDPAQGNPNEEVARFYAAAMSGAGPWAAWPFLLSLALMLLWLLS

SEQ ID NO: 70 - protein, 257 aa, synthetic construct
MAQRMTTQLLLLLVWVAVVGEAQTRIAWARTELLNVCMNAKHHKEKPGPEDKLHEQCRPWRKNACCSTNTSQEAHKDVSYLYRFNWNHCGEMAPACKRHFIQDTCLYECSPNLGPWIQQVDQSWRKERVLNVPLCKEDCQRWWEDCHTSHTCKSNWHKGWNWTSGFNKCAVGAACQPFHFYFPTPTVLCNEIWTHSYKVSNYSRGSGRCIQMWFDPAQGNPNEEVARFYAAAMSGAGPWAAWPFLLSLALMLLWLLS

SEQ ID NO: 71 - protein, 257 aa, synthetic construct
MAQRMTTQLLLLLVWVAVVGEAQTRIAWARTELLNVCMNAKHHKEKPGPEDKLHEQCRPWRKNACCSTNTSQEAHKDVSYLYRFNWNHCGEMAPACKRHFIQDTCLYECSPNLGPWIQQVDQSWRKERVLNVPLCKEDCEQWWEDCRTSYTCKSNWHKGWDWTSGVNKCPAGALCQPFHFYFPTPTVLCNEIWTHSYKVSNYSRGSGRCIQMWFDPAQGNPNEEVARFYAAAMSGAGPWAAWPFLLSLALMLLWLLS

SEQ ID NO: 72 - protein, 257 aa, synthetic construct
MAQRMTTQLLLLLVWVAVVGEAQTRIAWARTELLNVCMNAKHHKEKPGPEDKLHEQCRPWRKNACCSTNTSQEAHKDVSYLYRFNWNHCGEMAPACKRHFIQDTCLYECSPNLGPWIQQVDQSWRKERVLNVPLCKEDCEQWWEDCRTSYTCKSNWHKGWNWTSGFNKCAVGAACRTFESYFPTPTVLCNEIWTHSYKVSNYSRGSGRCIQMWFDPAQGNPNEEVARFYAAAMSGAGPWAAWPFLLSLALMLLWLLS

SEQ ID NO: 73 - protein, 257 aa, synthetic construct
MAQRMTTQLLLLLVWVAVVGEAQTRIAWARTELLNVCMNAKHHKEKPGPEDKLHEQCRPWRKNACCSTNTSQEAHKDVSYLYRFNWNHCGEMAPACKRHFIQDTCLYECSPNLGPWIQQVDQSWRKERVLNVPLCKEDCEQWWEDCRTSYTCKSNWHRGWNWTSGFNKCAVGAACQPFHFYFPTPAALCEGLWSHSYKVSNYSRGSGRCIQMWFDPAQGNPNEEVARFYAAAMSGAGPWAAWPFLLSLALMLLWLLS

SEQ ID NO: 74 - protein, 257 aa, synthetic construct
MAQRMTTQLLLLLVWVAVVGEAQTRIAWARTDLLNVCMDAKHHKEKPGPEDKLHEQCRPWRKNACCSTNTSQEAHKDVSYLYRFNWNHCGEMAPACKRHFIQDTCLYECSPNLGPWIQQVDQSWRKERVLNVPLCKEDCEQWWEDCRTSYTCKSNWHKGWNWTSGFNKCAVGAACQPFHFYFPTPTVLCNEIWTHSYKVSNYSRGSGRCIQMWFDPAQGNPNEEVARFYAAAMSGAGPWAAWPFLLSLALMLLWLLS

SEQ ID NO: 75 - protein, 257 aa, synthetic construct
MAQRMTTQLLLLLVWVAVVGEAQTRIAWARTELLNVCMNAKHHKTKPGPEDKLHEQCRPWRKNACCSTNTSQEAHKDVSYLYRFNWNHCGEMAPACKRHFIQDTCLYECSPNLGPWIQQVDQSWRKERVLNVPLCKEDCEQWWEDCRTSYTCKSNWHKGWNWTSGFNKCAVGAACQPFHFYFPTPTVLCNEIWTHSYKVSNYSRGSGRCIQMWFDPAQGNPNEEVARFYAAAMSGAGPWAAWPFLLSLALMLLWLLS

SEQ ID NO: 76 - protein, 257 aa, synthetic construct
MAQRMTTQLLLLLVWVAVVGEAQTRIAWARTELLNVCMNAKHHKEKPGPEDKLHDQCSPWKKNACCSTNTSQEAHKDVSYLYRFNWNHCGEMAPACKRHFIQDTCLYECSPNLGPWIQQVDQSWRKERVLNVPLCKEDCEQWWEDCRTSYTCKSNWHKGWNWTSGFNKCAVGAACQPFHFYFPTPTVLCNEIWTHSYKVSNYSRGSGRCIQMWFDPAQGNPNEEVARFYAAAMSGAGPWAAWPFLLSLALMLLWLLS

SEQ ID NO: 77 - protein, 257 aa, synthetic construct
MAQRMTTQLLLLLVWVAVVGEAQTRIAWARTELLNVCMNAKHHKEKPGPEDKLHEQCRPWRKNACCSTNTSQEAHKDVSYLYRFNWNHCGEMAPACKRHFIQDTCLYECSPNLGPWIQQVDQSWRKERVLNVPLCKEDCQQWWEDCRTSYTCKSNWHKGWNWTSGFNKCAVGAACQPFHFYFPTPTVLCNEIWTHSYKVSNYSRGSGRCIQMWFDPAQGNPNEEVARFYAAAMSGAGPWAAWPFLLSLALMLLWLLS

SEQ ID NO: 78 - protein, 257 aa, synthetic construct
MAQRMTTQLLLLLVWVAVVGEAQTRIAWARTELLNVCMNAKHHKEKPGPEDKLHEQCRPWRKNACCSTNTSQEAHKDVSYLYRFNWNHCGEMAPACKRHFIQDTCLYECSPNLGPWIQQVDQSWRKERVLNVPLCKEDCERWWEDCRTSYTCKSNWHKGWNWTSGFNKCAVGAACQPFHFYFPTPTVLCNEIWTHSYKVSNYSRGSGRCIQMWFDPAQGNPNEEVARFYAAAMSGAGPWAAWPFLLSLALMLLWLLS

SEQ ID NO: 79 - protein, 257 aa, synthetic construct
MAQRMTTQLLLLLVWVAVVGEAQTRIAWARTELLNVCMNAKHHKEKPGPEDKLHEQCRPWRKNACCSTNTSQEAHKDVSYLYRFNWNHCGEMAPACKRHFIQDTCLYECSPNLGPWIQQVDQSWRKERVLNVPLCKEDCEQWWEDCHTSYTCKSNWHKGWNWTSGFNKCAVGAACQPFHFYFPTPTVLCNEIWTHSYKVSNYSRGSGRCIQMWFDPAQGNPNEEVARFYAAAMSGAGPWAAWPFLLSLALMLLWLLS

SEQ ID NO: 80 - protein, 257 aa, synthetic construct
MAQRMTTQLLLLLVWVAVVGEAQTRIAWARTELLNVCMNAKHHKEKPGPEDKLHEQCRPWRKNACCSTNTSQEAHKDVSYLYRFNWNHCGEMAPACKRHFIQDTCLYECSPNLGPWIQQVDQSWRKERVLNVPLCKEDCEQWWEDCRTSHTCKSNWHKGWNWTSGFNKCAVGAACQPFHFYFPTPTVLCNEIWTHSYKVSNYSRGSGRCIQMWFDPAQGNPNEEVARFYAAAMSGAGPWAAWPFLLSLALMLLWLLS

SEQ ID NO: 81 - protein, 255 aa, synthetic construct
MVWKWMPLLLLLVCVATMCSAQDRTDLLNVCMDAKHHKTKPGPEDKLHDQCSPWKKNACCTASTSQELHKDTSRLYNFNWDHCGKMEPACKRHFIQDTCLYECSPNLGPWIQQVNQSWRKERFLDVPLCKEDCQQWWEDCHTSHTCKSNWHRGWDWTSGVNKCPAGALCRTFESYFPTPAALCEGLWSHSYKVSNYSRGSGRCIQMWFDSAQGNPNEEVARFYAAAMHVNAGEMLHGTGGLLLSLALMLQLWLLG

SEQ ID NO: 82 - protein, 159 aa, synthetic construct
EVQLVESGGGLVQPGGSLRLSCAASGSGFSGILMGWYRQAPGKQRELVATVTSHGTTNYADSVKGRFTISRPGAKNTVYLQMNSLRPEDTAVYYCNAHFPGIYWGKGTLVTVSSAAADYKDHDGDYKDHDIDYKDDDDKGAAHHHHHHGGGGSRRPYIL

SEQ ID NO: 83 - protein, 159 aa, synthetic construct
EVQLVESGGGLVQPGGSLRLSCAASGSGFSGPLMGWYRQAPGKQRELVATVTSHGTTNYADSVKGRFTISRPGAKNTVYLQMNSLRPEDTAVYYCNAHFPGIYWGKGTLVTVSSAAADYKDHDGDYKDHDIDYKDDDDKGAAHHHHHHGGGGSRRPYIL

SEQ ID NO: 84 - protein, 123 aa, synthetic construct; identical to SEQ ID NO: 45

SEQ ID NO: 85 - protein, 159 aa, synthetic construct
EVQLVESGGGLVQPGGSLRLSCAASESPFSGIIMGWYRQAPGKQRELVATVTSHGTTNYADSVKGRFTISREGAKNTVYLQMNSLRPEDTAVYYCNAHFPGIYWGKGTLVTVSSAAADYKDHDGDYKDHDIDYKDDDDKGAAHHHHHHGGGGSRRPYIL

SEQ ID NO: 86 - protein, 159 aa, synthetic construct
EVQLVESGGGLVQPGGSLRLSCAASESPFSGPIMGWYRQAPGKQRELVATVTSHGTTNYADSVKGRFTISREGAKNTVYLQMNSLRPEDTAVYYCNAHFPGIYWGKGTLVTVSSAAADYKDHDGDYKDHDIDYKDDDDKGAAHHHHHHGGGGSRRPYIL

SEQ ID NO: 87 - protein, 159 aa, synthetic construct
DVQLVESGGGLVQPGGSLRLSCAASGSGFSGMFMGWYRQAPGKQRELVATVTSHGTTNYADSVKGRFTISRDPAKNTVYLQMNSLRPEDTAVYYCNAHFPGIYWGKGTLVTVSSAAADYKDHDGDYKDHDIDYKDDDDKGAAHHHHHHGGGGSRRPYIL

SEQ ID NO: 88 - protein, 159 aa, synthetic construct
DVQLVESGGGLVQPGGSLRLSCAASESGFSGMFMGWYRQAPGKQRELVATVTSHGTTNYADSVKGRFTISRDNAKNTVYLQMNSLRPEDTAVYYCNAHFPGIYWGKGTLVTVSSAAADYKDHDGDYKDHDIDYKDDDDKGAAHHHHHHGGGGSRRPYIL

SEQ ID NO: 89 - protein, 159 aa, synthetic construct
DVQLVESGGGLVQPGGSLRLSCAASESPFSGMIMGWYRQAPGKQRELVATVTSHGTTNYADSVKGRFTISREGAKNTVYLQMNSLRPEDTAVYYCNAHFPGIYWGKGTLVTVSSAAADYKDHDGDYKDHDIDYKDDDDKGAAHHHHHHGGGGSRRPYIL

SEQ ID NO: 90 - protein, 159 aa, synthetic construct
DVQLVESGGGLVQPGGSLRLSCAASGSTFIGMIMGWYRQAPGKQRELVATVTSHGTTNYADSVKGRFTISRDNAKNTVYLQMNSLRPEDTAVYYCNAHFPGIYWGKGTLVTVSSAAADYKDHDGDYKDHDIDYKDDDDKGAAHHHHHHGGGGSRRPYIL

SEQ ID NO: 91 - protein, 159 aa, synthetic construct
DVQLVESGGGLVQPGGSLRLSCAASESGFSGMLMGWYRQAPGKQRELVATVTSHGTTNYADSVKGRFTISRDNAKNTVYLQMNSLRPEDTAVYYCNAHFPGIYWGKGTLVTVSSAAADYKDHDGDYKDHDIDYKDDDDKGAAHHHHHHGGGGSRRPYIL

SEQ ID NO: 92 - protein, 159 aa, synthetic construct
DVQLVESGGGLVQPGGSLRLSCAASGSGFSGMLMGWYRQAPGKQRELVATVTSHGTTNYADSVKGRFTISRDPAKNTVYLQMNSLRPEDTAVYYCNAHFPGIYWGKGTLVTVSSAAADYKDHDGDYKDHDIDYKDDDDKGAAHHHHHHGGGGSRRPYIL

SEQ ID NO: 93 - protein, 159 aa, synthetic construct
DVQLVESGGGLVQPGGSLRLSCAASESTFIGMLMGWYRQAPGKQRELVATVTSHGTTNYADSVKGRFTISRDNAKNTVYLQMNSLRPEDTAVYYCNAHFPGIYWGKGTLVTVSSAAADYKDHDGDYKDHDIDYKDDDDKGAAHHHHHHGGGGSRRPYIL

SEQ ID NO: 94 - protein, 159 aa, synthetic construct
DVQLVESGGGLVQPGGSLRLSCAASGSTFIGMIMGWYRQAPGKQRELVATVTSHGTTNYADSVKGRFTISRDPAKNTVYLQMNSLRPEDTAVYYCNAHFPGIYWGKGTLVTVSSAAADYKDHDGDYKDHDIDYKDDDDKGAAHHHHHHGGGGSRRPYIL

SEQ ID NO: 95 - protein, 159 aa, synthetic construct
DVQLVESGGGLVQPGGSLRLSCAASGSGFSGMIMGWYRQAPGKQRELVATVTSHGTTNYADSVKGRFTISRDNAKNTVYLQMNSLRPEDTAVYYCNAHFPGIYWGKGTLVTVSSAAADYKDHDGDYKDHDIDYKDDDDKGAAHHHHHHGGGGSRRPYIL

SEQ ID NO: 96 - protein, 140 aa, synthetic construct
QVQLQESGGGLVQAGGSLRVSCAASGFPFSTVYMSWVRQAPGKGLEWVSGINSNGGVIDYADSVKGRFTISRDNANNTLYLQLNSLKPEDTALYFCAKGRAFVATMPSSLRGQGTQVTVSSAAAYPYDVPDYGSHHHHHH

SEQ ID NO: 97 - protein, 255 aa, Mus musculus
MAHLMTVQLLLLVMWMAECAQSRATRARTELLNVCMDAKHHKEKPGPEDNLHDQCSPWKTNSCCSTNTSQEAHKDISYLYRFNWNHCGTMTSECKRHFIQDTCLYECSPNLGPWIQQVDQSWRKERILDVPLCKEDCQQWWEDCQSSFTCKSNWHKGWNWSSGHNECPVGASCHPFTFYFPTSAALCEEIWSHSYKLSNYSRGSGRCIQMWFDPAQGNPNEEVARFYAEAMSGAGFHGTWPLLCSLSLVLLWVIS

SEQ ID NO: 98 - protein, 123 aa, synthetic construct
EVQLVESGGGLVQPGGSLRLSCAASGSGFSGPIMGWYRQAPGKQRELVATVTSHGTTNYADSVKGRFTISRDNAKNTVYLQMNSLRPEDTAVYYCNAHFPGIYWGKGTLVTVSSAAAHHHHHH

SEQ ID NO: 99 - protein, 123 aa, synthetic construct
EVQLVESGGGLVQPGGSLRLSCAASGSGFSGIIMGWYRQAPGKQRELVATVTSHGTTNYADSVKGRFTISRDNAKNTVYLQMNSLRPEDTAVYYCNAHFPGIYWGKGTLVTVSSAAAHHHHHH

SEQ ID NO: 100 - protein, 148 aa, synthetic construct
EVQLVESGGGLVQPGGSLRLSCAVSGLGFSGIIMGWYRQAPGKQRELVATVTSHGTTNYADSVKGRFTISRDNAKNTVYLQMNSLRPEDTAVYYCNAHFPGIYWGKGTLVTVSSAAADYKDHDGDYKDHDIDYKDDDDKGAAHHHHHH

SEQ ID NO: 101 - protein, 148 aa, synthetic construct
EVQLVESGGGLVQPGGSLRLSCAVSGLGFSGIIMGWYRQAPGKQRELVATVTSHGTTNYADSVKGRFTISRDNAKDTVYLQMNSLRPEDTAVYYCNAHFPGIYWGKGTLVTVSSAAADYKDHDGDYKDHDIDYKDDDDKGAAHHHHHH

SEQ ID NO: 102 - protein, 148 aa, synthetic construct
EVQLVESGGGLVQPGGSLRLSCAVSGTGFSGIIMGWYRQAPGKQRELVATVTSHGTTNYADSVKGRFTISRDNAKDTVYLQMNSLRPEDTAVYYCNAHFPGIYWGKGTLVTVSSAAADYKDHDGDYKDHDIDYKDDDDKGAAHHHHHH

SEQ ID NO: 103 - protein, 148 aa, synthetic construct
EVQLVESGGGLVQPGGSLRLSCAVSGLGFSGPIMGWYRQAPGKQRELVATVTSHGTTNYADSVKGRFTISRDNAKNTVYLQMNSLRPEDTAVYYCNAHFPGIYWGKGTLVTVSSAAADYKDHDGDYKDHDIDYKDDDDKGAAHHHHHH

SEQ ID NO: 104 - protein, 148 aa, synthetic construct
EVQLVESGGGLVQPGGSLRLSCALSGAGFSGPIMGWYRQAPGKQRELVATVTSHGTTNYADSVKGRFTISRDNAKNTVYLQMNSLRPEDTAVYYCNAHFPGIYWGKGTLVTVSSAAADYKDHDGDYKDHDIDYKDDDDKGAAHHHHHH

SEQ ID NO: 105 - protein, 148 aa, synthetic construct
EVQLVESGGGLVQPGGSLRLSCALSGAGFSGPIMGWYRQAPGKQRELVATVTSHGTTNYADSVKGRFTISRDNAKETVYLQMNSLRPEDTAVYYCNAHFPGIYWGKGTLVTVSSAAADYKDHDGDYKDHDIDYKDDDDKGAAHHHHHH

SEQ ID NO: 106 - protein, 148 aa, synthetic construct
EVQLVESGGGLVQPGGSLRLSCAASGTGFSGIIMGWYRQAPGKQRELVATVTSHGTTNYADSVKGRFTISRDNAKNTVYLQMNSLRPEDTAVYYCNAHFPGIYWGKGTLVTVSSAAADYKDHDGDYKDHDIDYKDDDDKGAAHHHHHH

SEQ ID NO: 107 - protein, 148 aa, synthetic construct
EVQLVESGGGLVQPGGSLRLSCAASGTGFSGIIMGWYRQAPGKQRELVATVTSHGTTNYADSVKGRFTISRDNAKDTVYLQMNSLRPEDTAVYYCNAHFPGIYWGKGTLVTVSSAAADYKDHDGDYKDHDIDYKDDDDKGAAHHHHHH

SEQ ID NO: 108 - protein, 148 aa, synthetic construct
EVQLVESGGGLVQPGGSLRLSCAVSGLGFSGPIMGWYRQAPGKQRELVATVTSHGTTNYADSVKGRFTISRDNAKETVYLQMNSLRPEDTAVYYCNAHFPGIYWGKGTLVTVSSAAADYKDHDGDYKDHDIDYKDDDDKGAAHHHHHH

SEQ ID NO: 109 - protein, 123 aa, synthetic construct
EVQLVESGGGLVQPGGSLRLSCAASGSGFSGLIMGWYRQAPGKQRELVATVTSHGTTNYADSVKGRFTISRDNAKNTVYLQMNSLRPEDTAVYYCNAHFPGIYWGKGTLVTVSSAAAHHHHHH

SEQ ID NO: 110 - protein, 123 aa, synthetic construct
EVQLVESGGGLVQPGGSLRLSCAASESPFSGIIMGWYRQAPGKQRELVATVTSHGTTNYADSVKGRFTISREGAKNTVYLQMNSLRPEDTAVYYCNAHFPGIYWGKGTLVTVSSAAAHHHHHH

SEQ ID NO: 111 - protein, 123 aa, synthetic construct
EVQLVESGGGLVQPGGSLRLSCAASESPFSGPIMGWYRQAPGKQRELVATVTSHGTTNYADSVKGRFTISREGAKNTVYLQMNSLRPEDTAVYYCNAHFPGIYWGKGTLVTVSSAAAHHHHHH

SEQ ID NO: 112 - protein, 123 aa, synthetic construct
EVQLVESGGGLVQAGGSLRLSCAASGSGFSGIIMGWYRQAPGKQRELVATVTSHGTTNYADSVKGRFTISREDSKNTVLLQMNSLKPEDTAVYYCNAHFPGIYWGKGTLVTVSSAAAHHHHHH

SEQ ID NO: 113 - protein, 10 aa, synthetic construct
  VARIANT 1: note = G or E
  VARIANT 3: note = G, T, or P
  VARIANT 5: note = S or I
  VARIANT 8: note = I or L
XSXFXGMXMG

SEQ ID NO: 114 - protein, 25 aa, synthetic construct
  VARIANT 1: note = Q, E, D
  VARIANT 5: note = Q or V
  VARIANT 14: note = A or P
XVQLXESGGGLVQXGGSLRLSCAAS

SEQ ID NO: 115 - protein, 14 aa, synthetic construct
  VARIANT 5: note = V or A
WYRQXPGKQRELVA

SEQ ID NO: 116 - protein, 30 aa, synthetic construct
  VARIANT 7: note = D, E, or P
  VARIANT 8: note = N or G
  VARIANT 14: note = L or Y
  VARIANT 21: note = K or R
RFTISRXXAKNTVXLQMNSLXPEDTAVYYC

SEQ ID NO: 117 - protein, 11 aa, synthetic construct
  VARIANT 3: note = K or Q
  VARIANT 6: note = Q or L
WGXGTXVTVSS

SEQ ID NO: 118 - protein, 114 aa, synthetic construct
DVQLVESGGGLVQPGGSLRLSCAASESTFIGMLMGWYRQAPGKQRELVATVTSHGTTNYADSVKGRFTISRDNAKNTVYLQMNSLRPEDTAVYYCNAHFPGIYWGKGTLVTVSS

SEQ ID NO: 119 - protein, 114 aa, synthetic construct
EVQLVESGGGLVQPGGSLRLSCAASESTFIGMLMGWYRQAPGKQRELVATVTSHGTTNYADSVKGRFTISRDNAKNTVYLQMNSLRPEDTAVYYCNAHFPGIYWGKGTLVTVSS

SEQ ID NO: 120 - protein, 114 aa, synthetic construct
DVQLVESGGGLVQPGGSLRLSCAASESTFIGMLMGWYRQAPGKQRELVATVTSHGTTNYADSVKGRFTISREGAKNTVYLQMNSLRPEDTAVYYCNAHFPGIYWGKGTLVTVSS

SEQ ID NO: 121 - protein, 114 aa, synthetic construct
EVQLVESGGGLVQPGGSLRLSCAASESTFIGMLMGWYRQAPGKQRELVATVTSHGTTNYADSVKGRFTISREGAKNTVYLQMNSLRPEDTAVYYCNAHFPGIYWGKGTLVTVSS

(19) United States
(12) Patent Application Publication
(10) Pub. No.: US 2025/0304677 A1
(43) Pub. Date: Oct. 2, 2025

(54) Blood-Cerebrospinal Fluid Barrier Crossing Antibodies

(71) Applicants: VIB VZW, Gent (BE); Universiteit Gent, Gent (BE); Katholieke Universiteit Leuven, Leuven (BE)

(72) Inventors: Roosmarijn Vandenbroucke, Gent (BE); Florencia Linero, Gent (BE); Sophie Steeland, Gent (BE); Catelijne Stortelers, Gent (BE); Katrien Vanderheyden, Gent (BE); Nele Plehiers, Gent (BE)

(21) Appl. No.: 18/881,215

(22) PCT Filed: Jul. 4, 2023
(86) PCT No.: PCT/EP2023/068461

§ 371 Date: Jan. 3, 2025

(30) Foreign Application Priority Data
Jul. 4, 2022 (EP) 22182730.6
Aug. 31, 2022 (EP) 22193314.6

(51) Int. Cl. C07K 16/28

(52) U.S. Cl. CPC C07K 16/28; C07K 2317/22; C07K 2317/24; C07K 2317/33; C07K 2317/34; C07K 2317/569; C07K 2317/92

(57) ABSTRACT

The present invention relates to binding agents specifically binding to the folate transport complex. More specifically, antibodies or antibody fragments including immunoglobulin single variable domain (ISVD) antibodies are disclosed that bind the human folate receptor alpha (hFOLRα) present at the choroid plexus epithelial cells. The invention further relates to the antibodies and the methods herein described for use to increase the delivery of pharmaceutical compounds to the central nervous system via the process of receptor mediated endocytosis and/or transcytosis.

### CROSS REFERENCE TO RELATED APPLICATIONS

This application is a national phase entry under 35 U.S.C. § 371 of International Patent Application PCT/EP2023/068461, filed Jul. 4, 2023, designating the United States of America and published in English as International Patent Publication WO2024/008755 on Jan. 11, 2024, which claims the benefit under Article 8 of the Patent Cooperation Treaty to European Patent Application Serial No. 22182730.6, filed Jul. 4, 2022 and European Patent Application Serial No. 22193314.6, filed Aug. 31, 2022, the entireties of which are hereby incorporated by reference.

### INCORPORATION BY REFERENCE

The ST.26 XML Sequence listing named “10488-10706-US.XML”, created on Dec. 3, 2024, and having a size of 131,072 bytes, is hereby incorporated herein by this reference in its entirety.

### FIELD OF THE INVENTION

The present invention relates to binding agents specifically binding to the folate transport complex. More specifically, antibodies or antibody fragments including immunoglobulin single variable domain ^{∘}ISVD) antibodies are disclosed that bind the human folate receptor alpha ^{∘}hFOLRα) present at the choroid plexus epithelial cells. The invention further relates to the antibodies and the methods herein described for use to increase the delivery of pharmaceutical compounds to the central nervous system via the process of receptor mediated endocytosis and/or transcytosis.

### BACKGROUND

The development of central nervous system ^{∘}CNS) therapeutics has proven to be very challenging. New CNS drugs have historically suffered from considerably lower success rates during development than those for non-CNS indications. One of the major reasons is the presence of the blood-brain barrier ^{∘}BBB) proper, located at the endothelium of the cerebral microvessels, and the blood-cerebrospinal fluid barrier ^{∘}BCSFB). These blood-brain interfaces severely restrict the cerebral bioavailability of pharmaceutical compounds. Because of the limited penetration of for example antibodies or small molecules, high amounts of those compounds need to be administered to see some ^{∘}if any) effect. Besides the risk of high dosing on inducing peripheral side effects in the patient, it negatively impacts the cost to society. It also puts a pressure on the production capacities of for example antibodies, especially in larger indications such as Alzheimer's disease ^{∘}AD) and multiple sclerosis ^{∘}MS) with millions of patients, where the antibody production capacity can become an important limiting factor. While numerous attempts have been done to find means and methods to efficiently shuttle compounds over the BBB ^{∘}e.g. WO2015031673A2; WO2014033074A1; WO2015124540A1; WO2015191934A2), less work has been done on BCSFB crossers. The BCSFB is located at the choroid plexus, a highly vascularized structure protruding in the cerebrospinal fluid ^{∘}CSF) filled ventricles of the brain. It consists of a single layer of choroid plexus epithelial ^{∘}CPE) cells, surrounding stroma and fenestrated capillaries. The CPE cells are tightly connected with tight junctions and form the blood-CSF barrier which restricts the passage of molecules that can freely diffuse from the fenestrated capillaries into the stroma, towards the brain parenchyma. The cells are polarized and contain microvilli at the apical side and numerous infoldings at the basolateral side to increase the surface area with the CSF and plasma ultrafiltrate, respectively. The choroid plexus' most important functions next to the formation of a barrier is the transport of nutrients, ions, gases, proteins and metabolites between the fenestrated choroidal blood vessels and the CNS.

Transcytosis pathways ^{∘}e.g. via receptor mediated transcytosis) have raised considerable interest in the field of CNS delivery for their potential to deliver large cargoes including pharmacological agents. One of the possible targets found at the basolateral side of the CPE cells is the folate receptor α ^{∘}FRα) ^{∘}Grapp et al 2013 Nat Comm 4:2123; Strazielle & Ghersi-Egea 2016 Curr Pharmaceut Design 22: 5463-5476). It would thus be advantageous to highjack the folate transporting system at the BCSFB to increase the bioavailability of pharmacological compounds in the brain.

### SUMMARY

In current application single domain antibodies, more particularly VHHs, are disclosed that bind the human folate receptor alpha ^{∘}FRα), including the human FRα present at the CPE cells. The herein described antibodies may thus be applied to deliver compounds including therapeutic and/or diagnostic antibodies and small molecules across the BCSFB after a single systemic administration in mice.

Therefore, in a first aspect a folate receptor alpha ^{∘}FRα) binding agent capable of binding to human FRα with a dissociation constant k_{off} of less than 3×10⁻²/s, more particularly a koff of between 3×10⁻² and 1×10⁻³/s is provided. The koff is as determined by biolayer interferometry. In one embodiment, the binding agent specifically binds to the human FRα epitope comprising amino acid Q141 of SEQ ID NO: 1, more particularly binds an epitope on FRα which comprises at least one or more of the following residues, or all of the residues: R98, H99, E137, D138, Q141, E144, D145, R204, G205, Q211, W213, F214, D215, A217 and/or Q218 of SEQ ID NO: 1. In the present invention, those epitope binding ISVD are characterized in that they comprises a CDR3 sequence as depicted in SEQ ID No. 5. In another embodiment, the binding of the FRα binding agent to human FRα does not interfere with folate binding and/or folate transport by said human FRα. In another embodiment, the binding agent is capable of cross reacting with primate and mouse FRα. In addition, the present invention has revealed surprisingly that the FR3 region, more specifically the part of the so-called CDR4 loop, is important for the conformational requirements as to obtain BCSFB crossing, wherein said region is limited to those FRs wherein position 72 and 73 are defined as amino acids D, E, P and N, G, resp.

So in a particular embodiment, to provide for a BCSFB crossing agent, the ISVD comprises a paratope comprising amino acid residues F29, S30, G31 and I133 in CDR1, and T52, S53, H54 and T56 in CDR2, and H95, F96, P97, G98, I101, and Y102 in CDR3, and/or D72 and/or N73 in CDR4 according to Kabat numbering.

In a particular embodiment, any of the FRα binding agents listed above is also provided to facilitate, enable or improve the uptake of a biological or chemical entity to which it is coupled into the cerebrospinal fluid ^{∘}CSF) across the blood CSF barrier ^{∘}BCSFB). In another particular embodiment, the FRα binding agents also facilitates transport of a moiety to which it is coupled into FRα expressing cancer cells or improves the binding of the moiety to FRα expressing cancer cells. In another particular embodiment, the FRα binding agent comprises or consists of an immunoglobulin single variable domain or VHH.

In a second aspect, a blood-central nervous system ^{∘}CNS)-barrier shuttle is provided comprising any of said above FRα binding agents and in a third aspect, any of the FRα binding agents and any of the blood CNS barrier shuttles described above are provided for use as a medicament, more particularly for use in transporting one or more compounds to the CNS, more particularly across the BCSFB. Any of the FRα binding agents and any of the blood CNS barrier shuttles described above are also provided for use in treating a neurological disorder. In a particular embodiment, the neurological disorder is selected from the list consisting of Alzheimer's disease, stroke, dementia, muscular dystrophy, multiple sclerosis, amyotrophic lateral sclerosis, Charcot-Marie-Tooth disease, dystonia, Parkinson's disease, viral or microbial infections, inflammation, brain cancer, neuropathic pain and traumatic brain injury.

In a fourth aspect, a composition for use in the treatment or diagnosis of a neurological disorder is provided, the composition comprising a human FRα binding agent coupled to a neurological disorder drug or an imaging compound, wherein the composition binds the human FRα with a dissociation constant koff of less than 3×10⁻²/s as determined by biolayer interferometry, more particular with a koff of between 3×10⁻² and 1×10⁻³/s. In a particular embodiment, said neurological disorder drug is a biological, small molecule, therapeutic agent, an antisense oligonucleotide or test compound. In one embodiment, said binding to human FRα does not interfere with folate binding and/or transport by said human FRα. In another embodiment, the human FRα binding agent from said composition is capable of cross reacting with primate and mouse FRα. In yet another embodiment, said human FRα binding agent recognizes the same epitope in the human FRα as the FRα binding agent consisting of the sequence as depicted in SEQ ID No. 2. In another particular embodiment, said composition is a multispecific antibody comprising said human FRα binding agent and a second antigen binding site which binds a brain antigen. In a more particular embodiment, said brain antigen is selected from the group consisting of beta-secretase 1 ^{∘}BACE1), amyloid beta, epidermal growth factor receptor ^{∘}EGFR), human epidermal growth factor receptor 2 ^{∘}HER2), Tau, apolipoprotein E4 ^{∘}ApoE4), alpha-synuclein, CD20, huntingtin, prion protein ^{∘}PrP), leucine rich repeat kinase 2 ^{∘}LRRK2), parkin, presenilin 1, presenilin 2, gamma secretase, death receptor 6 ^{∘}DR6), amyloid precursor protein ^{∘}APP), p75 neurotrophin receptor ^{∘}p75NTR) and caspase 6. In yet another embodiment, the FRα binding agent from the composition comprises or consists of an immunoglobulin single variable domain or VHH.

### Note

The project leading to this application has received funding from the European Union's Horizon 2020 research and innovation programme under grant agreement No 721058.

### BRIEF DESCRIPTION OF THE FIGURES

The patent or application file contains at least one drawing executed in color. Copies of this patent or patent application publication with color drawing^{∘}s) will be provided by the Office upon request and payment of the necessary fee.

The drawings described are only schematic and are non-limiting. In the drawings, the size of some of the elements may be exaggerated and not drawn on scale for illustrative purposes.

FIGS. 1A-1E shows the in vitro characterization of anti-folate receptor alpha ‘FRα’ or ‘FOLR1’, as used interchangeably herein) VHHs from the different CDR3 families; ^{∘}FIG. 1A and FIG. 1B) Determination of the binding affinity of anti-FOLR1 VHHs for recombinant mouse ^{∘}mFOLR1, FIG. 1A) and human ^{∘}hFOL1, FIG. 1B) FOLR1 using ELISA. Data were normalized and technical replicates were plotted as mean±SD. Data were fitted by non-linear regression and the half-maximal effective concentration ^{∘}EC50) was calculated using GraphPad Prism 9 software. ^{∘}FIG. 1C and FIG. 1D) Binding affinity determination of anti-FOLR1 VHHs for native FOLR1. Mouse ^{∘}FIG. 1C) or human FOLR1 FIG. 1D) was overexpressed on HEK293T cells and binding of the anti-FOLR1 VHHs was assessed using flow cytometry. The mean fluorescence intensity ^{∘}MFI) of the phycoerythrin signal was plotted for each VHH concentration. Data were fitted by non-linear regression using GraphPad Prism 9 software. ^{∘}FIG. 1E) Determination of the competition of representative family members with folic acid ^{∘}FA) using ELISA. Mouse FOLR1 was coated on the plate and was pre-incubated with or without 100 μM FA before adding the VHHs. Data were fitted by non-linear regression using GraphPad Prism 9 software.

FIGS. 2A-2C illustrates the epitope mapping of anti-FOLR1 VHHs on human FOLR1/2 chimeras. ^{∘}FIG. 2A) To narrow down the epitope of the anti-folate receptor alpha ^{∘}FOLR1) VHHs, chimeric constructs were generated by exchanging a sequence block in human FOLR1 ^{∘}hFOLR1) by the corresponding human folate receptor beta ^{∘}hFOLR2) sequence, since the VHHs are able to bind both hFOLR1 and mouse FOLR1 ^{∘}mFOLR1; SEQ ID No.97), but not hFOLR2. These regions are indicated by the green arrows. Chimera 1 SEQ ID No. 67) and 7 ^{∘}Ch7; SEQ ID No. 73) also contain an additional residue change at a separate location. Identical residues as the reference hFOLR1 are displayed as dots. ^{∘}FIG. 2B, FIG. 2C) Binding of the anti-FOLR1 VHHs to the chimeric constructs was assessed via flow cytometry. HEK293T cells were transfected with hFOLR1, hFOLR2, one of the hFOLR1/2 chimeric constructs ^{∘}FIG. 2B) or single residue mutants ^{∘}FIG. 2C). Binding of VHHs 2HFO19, 2HFO42, 3MFR60, and a mouse anti-FOLR1 monoclonal antibody ^{∘}mAb) was detected using a mouse anti-His tag antibody for the VHHs followed by an anti-mouse-phycoerythrin antibody. All VHHs were tested at a concentration of 200 nM and untransfected cells were used as a negative control. The colors used for the VHHs represent the different CDR3 families; blue: family 1, green: family 3, grey: family 10.

FIGS. 3A-3D shows the in vivo blood-cerebrospinal fluid ^{∘}CSF) barrier crossing capacity of anti-FOLR1 VHHs. ^{∘}FIG. 3A) To study the in vivo crossing potential, the VHHs were fused to neurotensin ^{∘}NT). NT as such cannot enter the central nervous system ^{∘}CNS), but causes a measurable drop in temperature upon engagement of neuronal NT receptor ^{∘}NTSR) 1 and astrocytic NTSR2 in the CNS. A non-blood-CSF barrier crossing VHH fused to NT thus will not cause a drop in body temperature after peripheral administration, whereas a VHH that can cross the blood-CSF barrier will induce hypothermia. ^{∘}FIG. 3B) VHHs from the different anti-folate receptor alpha ^{∘}FOLR1) VHH families or an irrelevant anti-eGFP VHH were injected intravenously ^{∘}iv) in TLR4−/− mice at a dose of 250 nmol/kg. The body temperature of the mice was measured continuously after injection using intraperitoneally ^{∘}ip) implanted TA-F10 telemetry probes and Dataquest Art software ^{∘}Data Sciences International). The plotted data points represent the average change in body temperature every 15 min and biological replicates ^{∘}n=2-10) are displayed as median±95% confidence interval. The dotted red line represents the lowest average value of the anti-eGFP control VHH. The colors of the graphs represent the different CDR3 families; blue: family 1, green: family 3, grey: family 10. ^{∘}FIGS. 3C and 3D) Determination of the dose-response of 2HFO42 ^{∘}FIG. 3C) and the control VHH anti-eGFP ^{∘}D). TLR4−/− mice were injected iv with different doses of the VHHs followed by continuous monitoring of the body temperature using ip implanted TA-F10 telemetry probes and Dataquest Art software ^{∘}Data Sciences International). The average temperature change was plotted every 15 min and biological replicates ^{∘}n=2-3) are displayed as mean±SD. The dotted red line represents the lowest average value of the anti-eGFP control VHH. Figure partially created with Biorender.com.

FIGS. 4A-4C illustrates the in vivo blood-cerebrospinal fluid ^{∘}CSF) barrier crossing capacity of 2HFO9 and 2MFR67 mutants. ^{∘}FIG. 4A) Sequence alignment of family 3 members 2HFO42, 2HFO9, and 2MFR67. Identical residues as the reference 2HFO42 are displayed as dots. ^{∘}FIGS. 4B and 4C) Determination of the blood-CSF barrier crossing of 2HFO9 and 2MFR67 mutants. TLR4−/− mice were injected intravenously with mutant forms of 2HFO9 ^{∘}n=2-10) ^{∘}FIG. 4B) or 2MFR67 ^{∘}n=2-4) ^{∘}FIG. 4C) fused to neurotensin ^{∘}NT) at a dose of 250 nmol/kg followed by continuous monitoring of the body temperature using intraperitoneally implanted TA-F10 telemetry probes and Dataquest Art software ^{∘}Data Sciences International). The average temperature change was plotted every 15 min and biological replicates are displayed as median±95% confidence interval ^{∘}FIG. 4B) or mean±SD ^{∘}FIG. 4C). The dotted red line represents the lowest average value of the anti-eGFP control VHH.

FIGS. 5A and 5B shows the fold change in the off-rates of 2HFO9 and 2MFR67 mutants. The off-rates of the 2HFO9 ^{∘}FIG. 5A) and 2MFR67 ^{∘}FIG. 5B) mutants and the three wild-type ^{∘}WT) VHHs from family 3 were determined by biolayer interferometry ^{∘}BLI) on both mouse and human biotinylated folate receptor alpha ^{∘}FOLR1) that was immobilized on streptavidin biosensors. The off-rates were calculated using a 1:1 binding model. The fold change compared to the off-rates of the corresponding WT VHH is represented as the WT VHH off-rate divided by the off-rate of the mutant for the VHHs that show an improved off-rate compared to the WT. For the VHHs that show a worse off-rate compared to the WT, the fold change is represented as the negative value of the mutant VHH off-rate divided by the WT off-rate. The red bars represent the WT 2HFO9 and 2MFR67 in the upper and lower panel respectively.

FIGS. 6A-6E shows the plasma and cerebrospinal fluid ^{∘}CSF) kinetics in non-human primate. A rhesus macaque was injected intravenously with a 1:1 mixture of 2HFO42 ^{∘}A-B) and control VHH or of 2MFR67 ^{∘}FIGS. 6C-6E) and control VHH ^{∘}8 mg/kg each). Plasma ^{∘}FIGS. 6A-6C) and CSF ^{∘}FIGS. 6B-6D) were sampled before injection, 5 min and 30 min after injection followed by hourly sampling up to 7 h after injection. Final samples were taken 24 h after injection. VHH levels were determined by ELISA using a rabbit anti-cMyc tag antibody or a mouse anti-His tag antibody for 2HFO42, 2MFR67 or control VHHs respectively. A standard curve was generated for all VHHs after fitting by non-linear regression and VHH levels in the samples were interpolated based on this standard curve using GraphPad Prism 9 software and the CSF/plasma ratio ^{∘}FIG. 6E) was calculated. Technical replicates are displayed as mean±SD. The 24 h plasma samples were below the lower limit of quantification and are not displayed on the graph.

FIG. 7 shows the inhibition of VHHs for binding of 2MFR67 VHH to human FRα in competition ALPHALISA.

FIGS. 8A and 8B illustrates the binding of 2HFO42 family 3 VHHs to mouse ^{∘}FIG. 8A) and cynomolgus ^{∘}FIG. 8B) FRα in ELISA using anti-VHH detection.

FIGS. 9A and 9B shows the screening of affinity optimized libraries of the CDR1, CDR2, CDR4 and combinatorial library in competition AlphaLISA on human FRα ^{∘}FIG. 9A) and off-rate analysis of variants with single amino acid substitutions in the CDR1, CDR2, or FR 3/CDR4 regions ^{∘}FIG. 9B). Non-redundant clones were tested as crude periplasmatic extracts for binding analysis to human FRα and mouse FRα proteins captured on streptavidin biosensors in BLI. Variants P0150001 and P0150005 grown in the same 96-well plates were included as controls.

FIG. 10A shows the binding of the lead panel of affinity optimized variants to human and mouse FOLR1, as the average equilibrium dissociation constants ^{∘}Kdis) of purified affinity optimized 2HF042 ^{∘}family 3) variants for binding to human FRα ^{∘}hu) and mouse FRα ^{∘}ms) in BLI. Purified VHHs ^{∘}100 nM) were analysed for binding to avi-tagged biotinylated FRα protein captured on streptavidin-biosensors via BLI.

FIG. 10B shows the binding of affinity optimized variants to cell-expressed human FOLR1. Example of dose-dependent binding of affinity optimized variants of 2HFO42 to HeLa ^{∘}cervix carcinoma) cells that endogenously express human FOLR1. EC₅₀ values are found in Table 9. Detection of VHH cell binding was done through anti-Flag M2 mIgG1 followed by anti-mFc-PE in flow cytometry. Results are depicted as mean fluorescence intensity ^{∘}MFI).

FIGS. 11A-11D shows the amount of VHH collected in the apical compartment during the in vitro transcytosis assay at different timepoints on HIBCPP cells ^{∘}FIG. 11A). Representative transcytosis assay in the inverse setup with different 2HFO42 variants tested at 100 nM for 4 hours. VHH levels are shown as normalised values to the anti-TFR NB188 ^{∘}P01500022) that was included as system control are shown, with the TEER values of the respective inserts shown on the right Y-axis ^{∘}FIG. 11B). Comparison of transcytosis of different affinity optimised 2HFO42 variants across assays, with inserts of TEER values between 500-900 Ohm ^{∘}FIG. 11C) and TEER values higher than 900 Ohm, ^{∘}FIG. 11D).

FIGS. 12A and 12B—FIG. 12A. shows the interface of family 3 VHH 2HF042 ^{∘}Nb42) with human FRα protein determined in co-crystal structure. FIG. 12B. Zoom-in of the hydrogen-bonding network surrounding FRα Q141.

FIG. 13 shows the interface of family 1 VHH 2HF019 ^{∘}Nb19) with human FRα protein determined in co-crystal structure. Zoom-in of the hydrogen-bonding network surrounding FRα Q141.

FIGS. 14A and 14B illustrates the in vivo blood-cerebrospinal fluid ^{∘}CSF) barrier crossing capacity of P01500024, P01500031 and P01500032. ^{∘}FIGS. 14A and 14B) Determination of the blood-CSF barrier crossing of P01500024, P01500031 and P01500032 ^{∘}all with short NT8-13) compared to 2HFO24 with long ^{∘}NT) and short NT ^{∘}NT8-13). TLR4−/− mice were injected intravenously with VHHs fused to neurotensin ^{∘}NT) at a dose of 250 nmol/kg followed by continuous monitoring of the body temperature using intraperitoneally implanted TA-F10 telemetry probes and Dataquest Art software Data Sciences International). The average temperature change was plotted every 15 min and biological replicates are displayed as mean±SD. The dotted red ^{∘}lower) line represents the lowest average value of the anti-eGFP control VHH.

FIG. 15 shows the binding to FRα expressing breast cancer derived cell lines. Quantification of dot blot results of VHHs on human FOLR1-expressing MCF7 and MDA-MB-231 breast tumor derived cell lysates revealed that the family 1 and family 3 FRα binding VHHs showed improved binding to MCF7 and MDA-MD-231 cells as compared to the positive control antibody.

FIG. 16 shows the co-crystal structure of 2MFR67 with human FOLR1.

FIG. 17 shows the overlay of the 2HFO42 ^{∘}Nb42)-FOLR1 and 2MFR67 ^{∘}Nb67)-FOLR1 crystals structures. The orientation of the CDR4 region in 2MFR67 is directed away from the CDR1-3 regions in an open conformation, whereas this CDR4 region in 2HFO42 is flexible and found in closed and open conformations ^{∘}arrow).

FIG. 18 shows a comparison of the FOLR1 binding sites of 2HFO19 ^{∘}Nb19, black) and 2HFO42 ^{∘}Nb42, gray). The CDR3 region of 2HFO42 makes additional interactions with FOLR1 residues 204, 213-218 ^{∘}C), compared to 2HFO19.

FIGS. 19A and 19B shows the ITC of humanised 2HFO42 ^{∘}P0150001, upper panel) and 2MFR67 ^{∘}P01500005, lower panel) with hFRα.

FIG. 20 demonstrates that the affinity optimized variant P01500076 is inducing hypothermia in human FRa tg/tg mice ^{∘}upper panel), but not in TLR4 KO mice that express mouse FOLR1 lower panel).

FIG. 21 shows the in vivo blood-cerebrospinal fluid ^{∘}CSF) barrier crossing capacity of 2HFO42-NT, compared to 2HFO42-^{∘}eGFP)-NT8-13^{∘}=2HFO42 Nb fused to a Nb against eGFP fused to Neurotensin) ^{∘}A) and bivalent 2HFO42-2HFO42-NT ^{∘}B) in TLR4 KO mice.

FIG. 22 provides the CDR1-3 annotations according to MacCallum, AbM, Chothia, Kabat, IMGT, and the CDR sequences as annotated herein, in grey labeled boxes, as well as the ‘CDR4’ or FR3 loop region, referred to in the application, as for the sequence of VHH 2HFO42 ^{∘}SEQ ID No.2). A similar annotation for all other VHH sequences and sequence variants can be performed by the skilled person based on this template and/or by aligning to SEQ ID No.2.

### DETAILED DESCRIPTION

### Definitions

In order that the present description can be more readily understood, certain terms are first defined. Additional definitions are set forth throughout the detailed description. The present invention is described with respect to particular embodiments and with reference to certain drawings but the invention is not limited thereto but only by the claims. Any reference signs in the claims shall not be construed as limiting the scope. The drawings described are only schematic and are non-limiting. In the drawings, the size of some of the elements may be exaggerated and not drawn on scale for illustrative purposes. It is to be noted that the term “a” or “an” entity refers to one or more of that entity; for example, “a nucleotide sequence”, is understood to represent one or more nucleotide sequences. As such, the terms “a” or “an”), “one or more” and “at least one” can be used interchangeably herein. Furthermore, “and/or” where used herein is to be taken as specific disclosure of each of the two specified features or components with or without the other. Thus, the term “and/or” as used in a phrase such as “A and/or B” herein is intended to include “A and B”, “A or B”, “A” ^{∘}alone), and “B” ^{∘}alone). Likewise, the term “and/or” as used in a phrase such as “A, B, and/or C” is intended to encompass each of the following aspects: A, B, and C; A, B, or C; A or C; A or B; B or C; A and C; A and B; B and C; A ^{∘}alone); B ^{∘}alone); and C ^{∘}alone). Where an indefinite or definite article is used when referring to a singular noun e.g. “a” or “an”, “the”, this includes a plural of that noun unless something else is specifically stated. Furthermore, the terms first, second, third and the like in the description and in the claims, are used for distinguishing between similar elements and not necessarily for describing a sequential or chronological order. It is to be understood that the terms so used are interchangeable under appropriate circumstances and that the embodiments of the invention described herein are capable of operation in other sequences than described or illustrated herein.

It is understood that wherever aspects or embodiments are described herein with the language “comprising”, otherwise analogous aspects or embodiments described in terms of “consisting of” and/or “consisting essentially of” are also provided. Where the term “comprising” is used in the present description and claims, it does not exclude other elements or steps. Unless specifically defined herein, all terms used herein have the same meaning as they would to one skilled in the art of the present invention.

Unless defined otherwise, all technical and scientific terms used herein have the same meaning as commonly understood by one of ordinary skill in the art to which this disclosure is related. For example, the Concise Dictionary of Biomedicine and Molecular Biology, Juo, Pei-Show, 2nd ed., 2002, CRC Press; The Dictionary of Cell and Molecular Biology, 3rd ed., 1999, Academic Press; and the Oxford Dictionary of Biochemistry and Molecular Biology, Revised, 2000, Oxford University Press, provide one of skill with a general dictionary of many of the terms used in this disclosure. Practitioners are particularly directed to Sambrook et al., Molecular Cloning: A Laboratory Manual, 4th ed., Cold Spring Harbor Press, Plainsview, New York ^{∘}2012); and Ausubel et al., current Protocols in Molecular Biology ^{∘}Supplement 100), John Wiley & Sons, New York ^{∘}2012), for definitions and terms of the art. The definitions provided herein should not be construed to have a scope less than understood by a person of ordinary skill in the art.

Units, prefixes, and symbols are denoted in their Système International de Unites ^{∘}SI) accepted form. Numeric ranges are inclusive of the numbers defining the range. Unless otherwise indicated, nucleotide sequences are written left to right in 5′ to 3′ orientation. Amino acid sequences are written left to right in amino to carboxy orientation. The headings provided herein are not limitations of the various aspects of the disclosure, which can be had by reference to the specification as a whole. Accordingly, the terms defined immediately below are more fully defined by reference to the specification in its entirety.

The term “about” is used herein to mean approximately, roughly, around, or in the regions of. When the term “about” is used in conjunction with a numerical range, it modifies that range by extending the boundaries above and below the numerical values set forth. In general, the term “about” can modify a numerical value above and below the stated value by a variance. For example a dissociation constant koff of about 1.50×10⁻²/s implies that the koff is within the range between 1.45×10⁻² to 1.55×10⁻²/s.

The present application relates to antibodies binding the mouse, primate and human folate receptor.

The term “antibody” as used herein, refers to an immunoglobulin ^{∘}Ig) molecule or a molecule comprising an immunoglobulin ^{∘}Ig) domain, which specifically binds with an antigen. “Antibodies” can be intact immunoglobulins derived from natural sources or from recombinant sources and can be immunoreactive portions of intact immunoglobulins. Antibodies are typically tetramers of immunoglobulin molecules. The term “immunoglobulin ^{∘}Ig) domain” as used herein refers to a globular region of an antibody chain, or to a polypeptide that essentially consists of such a globular region. Immunoglobulin domains are characterized in that they retain the immunoglobulin fold ^{∘}Ig fold as named herein) characteristic of antibody molecules, which consists of a two-layer sandwich of about seven to nine antiparallel β-strands arranged in two β-sheets, optionally stabilized by a conserved disulphide bond. The term “immunoglobulin ^{∘}Ig) domain”, includes “immunoglobulin constant domain”, and “immunoglobulin variable domain” ^{∘}abbreviated as “IVD”), wherein the latter means an immunoglobulin domain essentially consisting of four “framework regions” which are referred to in the art and herein below as “framework region 1” or “FR1”; as “framework region 2” or “FR2”; as “framework region 3” or “FR3”; and as “framework region 4” or “FR4”, respectively; which framework regions are interrupted by three “complementarity determining regions” or “CDRs”, which are referred to in the art and herein below as “complementarity determining region 1” or “CDR1”; as “complementarity determining region 2” or “CDR2”; and as “complementarity determining region 3” or “CDR3”, respectively. Thus, the general structure or sequence of an immunoglobulin variable domain can be indicated as follows: FR1-CDR1-FR2-CDR2-FR3-CDR3-FR4. It is the immunoglobulin variable domain's) ^{∘}IVDs) that confer specificity to an antibody for the antigen by carrying the antigen-binding site.

Determination of CDR regions may be done according to different methods, such as the designation based on contact analysis and binding site topography as described in MacCallum et al. ^{∘}J. Mol. Biol. ^{∘}1996) 262, 732-745). Or alternatively the annotation of CDRs may be done according to AbM ^{∘}AbM is Oxford Molecular Ltd.'s antibody modelling package as described on http://www.bioinf.org.uk/abs/index.html), Chothia ^{∘}Chothia and Lesk, 1987; Mol Biol. 196:901-17), Kabat ^{∘}Kabat et al., 1991; 5^{th} edition, NIH publication 91-3242), and IMGT ^{∘}LeFranc, 2014; Frontiers in Immunology. 5 ^{∘}22): 1-22). Said annotations further include delineation of CDRs and framework regions ^{∘}FRs) in immunoglobulin-domain-containing proteins, and are known methods and systems to a skilled artisan who thus can apply these annotations onto any immunoglobulin protein sequences without undue burden. These annotations differ slightly, but each intend to comprise the regions of the loops involved in binding the target.

An “immunoglobulin domain” of this application also includes “immunoglobulin single variable domains” ^{∘}abbreviated as “ISVD”), equivalent to the term “single variable domains”, and defines molecules wherein the antigen binding site is present on, and formed by, a single immunoglobulin domain. This sets immunoglobulin single variable domains apart from “conventional” immunoglobulins or their fragments, wherein two immunoglobulin domains, in particular two variable domains, interact to form an antigen binding site. Typically, in conventional immunoglobulins, a heavy chain variable domain ^{∘}VH) and a light chain variable domain ^{∘}VL) interact to form an antigen binding site. In this case, the complementarity determining regions ^{∘}CDRs) of both VH and VL will contribute to the antigen binding site, i.e. a total of 6 CDRs will be involved in antigen binding site formation. In view of the above definition, the antigen-binding domain of a conventional 4-chain antibody ^{∘}such as an IgG, IgM, IgA, IgD or IgE molecule; known in the art) or of a Fab fragment, a F^{∘}ab′)2 fragment, an Fv fragment such as a disulphide linked Fv or a scFv fragment, or a diabody ^{∘}all known in the art) derived from such conventional 4-chain antibody, would normally not be regarded as an immunoglobulin single variable domain, as, in these cases, binding to the respective epitope of an antigen would normally not occur by one ^{∘}single) immunoglobulin domain but by a pair of ^{∘}associated) immunoglobulin domains such as light and heavy chain variable domains, i.e., by a VH-VL pair of immunoglobulin domains, which jointly bind to an epitope of the respective antigen. In contrast, immunoglobulin single variable domains are capable of specifically binding to an epitope of the antigen without pairing with an additional immunoglobulin variable domain. The binding site of an immunoglobulin single variable domain is formed by a single VH/VHH or VL domain. Hence, the antigen binding site of an immunoglobulin single variable domain is formed by no more than three CDRs. As such, the single variable domain may be a light chain variable domain sequence ^{∘}e.g., a VL-sequence) or a suitable fragment thereof; or a heavy chain variable domain sequence ^{∘}e.g., a VH-sequence or VHH sequence) or a suitable fragment thereof; as long as it is capable of forming a single antigen binding unit ^{∘}i.e., a functional antigen binding unit that essentially consists of the single variable domain, such that the single antigen binding domain does not need to interact with another variable domain to form a functional antigen binding unit). In one embodiment of the invention, the immunoglobulin single variable domains are heavy chain variable domain sequences ^{∘}e.g., a VH-sequence); more specifically, the immunoglobulin single variable domains can be heavy chain variable domain sequences that are derived from a conventional four-chain antibody or heavy chain variable domain sequences that are derived from a heavy chain antibody. For example, the immunoglobulin single variable domain may be a ^{∘}single) domain antibody ^{∘}or an amino acid sequence that is suitable for use as a ^{∘}single) domain antibody), a “dAb” or dAb ^{∘}or an amino acid sequence that is suitable for use as a dAb) or a Nanobody ^{∘}as defined herein, and including but not limited to a VHH); other single variable domains, or any suitable fragment of any one thereof. In particular, the immunoglobulin single variable domain may be a Nanobody ^{∘}as defined herein) or a suitable fragment thereof. Note: Nanobody®, Nanobodies® and Nanoclone® are registered trademarks of Ablynx N.V. For a general description of Nanobodies, reference is made to the further description below, as well as to the prior art cited herein, such as e.g. described in WO2008/020079.

Immunoglobulin domains herein also include “VHH domains”, also known as VHHs, VHH domains, VHH antibody fragments, and VHH antibodies, have originally been described as the antigen-binding immunoglobulin ^{∘}Ig) ^{∘}variable) domain of “heavy chain antibodies” ^{∘}i.e., of “antibodies devoid of light chains”; Hamers-Casterman et al ^{∘}1993) Nature 363: 446-448). The term “VHH domain” has been chosen to distinguish these variable domains from the heavy chain variable domains that are present in conventional 4-chain antibodies ^{∘}which are referred to herein as “VH domains”) and from the light chain variable domains that are present in conventional 4-chain antibodies ^{∘}which are referred to herein as “VL domains”). For a further description of VHHs and Nanobody, reference is made to the review article by Muyldermans ^{∘}Reviews in Molecular Biotechnology 74: 277-302, 2001), as well as to the following patent applications, which are mentioned as general background art: WO 94/04678, WO 95/04079 and WO 96/34103 of the Vrije Universiteit Brussel; WO 94/25591, WO 99/37681, WO 00/40968, WO 00/43507, WO 00/65057, WO 01/40310, WO 01/44301, EP 1134231 and WO 02/48193 of Unilever; WO 97/49805, WO 01/21817, WO 03/035694, WO 03/054016 and WO 03/055527 of the Vlaams Instituut voor Biotechnologie ^{∘}VIB); WO 03/050531 of Algonomics N.V. and Ablynx N.V.; WO 01/90190 by the National Research Council of Canada; WO 03/025020^{∘}=EP 1433793) by the Institute of Antibodies; as well as WO 04/041867, WO 04/041862, WO 04/041865, WO 04/041863, WO 04/062551, WO 05/044858, WO 06/40153, WO 06/079372, WO 06/122786, WO 06/122787 and WO 06/122825, by Ablynx N.V. and the further published patent applications by Ablynx N.V. As described in these references, Nanobody ^{∘}in particular VHH sequences and partially humanized Nanobody) can in particular be characterized by the presence of one or more “Hallmark residues” in one or more of the framework sequences. A further description of the Nanobody, including humanization and/or camelization of Nanobody, as well as other modifications, parts or fragments, derivatives or “Nanobody fusions”, multivalent constructs ^{∘}including some non-limiting examples of linker sequences) and different modifications to increase the half-life of the Nanobody and their preparations can be found e.g. in WO 08/101985 and WO 08/142164.

“Domain antibodies”, also known as “Dabs”, “Domain Antibodies”, and “dAbs” ^{∘}the terms “Domain Antibodies” and “dAbs” being used as trademarks by the GlaxoSmithKline group of companies) have been described in e.g., EP 0368684, Ward et al. ^{∘}Nature 341: 544-546, 1989), Holt et al. ^{∘}Tends in Biotechnology 21: 484-490, 2003) and WO 03/002609 as well as for example WO 04/068820, WO 06/030220, WO 06/003388 and other published patent applications of Domantis Ltd. Domain antibodies essentially correspond to the VH or VL domains of non-camelid mammalians, in particular human 4-chain antibodies. In order to bind an epitope as a single antigen binding domain, i.e., without being paired with a VL or VH domain, respectively, specific selection for such antigen binding properties is required, e.g. by using libraries of human single VH or VL domain sequences. Domain antibodies have, like VHHs, a molecular weight of approximately 13 to approximately 16 kDa and, if derived from fully human sequences, do not require humanization for e.g. therapeutical use in humans. It should also be noted that single variable domains can be derived from certain species of shark ^{∘}for example, the so-called “IgNAR domains”, see for example WO 05/18629).

Immunoglobulin single variable domains such as Domain antibodies and Nanobody ^{∘}including VHH domains and humanized VHH domains), represent in vivo matured macromolecules upon their production, but can be further subjected to affinity maturation by introducing one or more alterations in the amino acid sequence of one or more CDRs, which alterations result in an improved affinity of the resulting immunoglobulin single variable domain for its respective antigen, as compared to the respective parent molecule. Affinity-matured immunoglobulin single variable domain molecules of the invention may be prepared by methods known in the art, for example, as described by Marks et al. ^{∘}Biotechnology 10:779-783, 1992), Barbas et al. ^{∘}Proc. Nat. Acad. Sci, USA 91: 3809-3813, 1994), Shier et al. ^{∘}Gene 169: 147-155, 1995), Yelton et al. ^{∘}Immunol. 155: 1994-2004, 1995), Jackson et al. ^{∘}J. Immunol. 154: 3310-9, 1995), Hawkins et al. ^{∘}J. Mol. Biol. 226: 889 896, 1992), Johnson and Hawkins ^{∘}Affinity maturation of antibodies using phage display, Oxford University Press, 1996). The process of designing/selecting and/or preparing a polypeptide, starting from an immunoglobulin single variable domain such as a Domain antibody or a Nanobody, is also referred to herein as “formatting” said immunoglobulin single variable domain; and an immunoglobulin single variable domain that is made part of a polypeptide is said to be “formatted” or to be “in the format of” said polypeptide. Examples of ways in which an immunoglobulin single variable domain can be formatted and examples of such formats for instance to avoid glycosylation will be clear to the skilled person based on the disclosure herein.

Immunoglobulin single variable domains such as Domain antibodies and Nanobody ^{∘}including VHH domains) can be subjected to humanization, i.e. increase the degree of sequence identity with the closest human germline sequence. In particular, humanized immunoglobulin single variable domains, such as Nanobody ^{∘}including VHH domains) may be immunoglobulin single variable domains that are as generally defined for in the previous paragraphs, but in which at least one amino acid residue is present ^{∘}and in particular, at least one framework residue) that is and/or that corresponds to a humanizing substitution ^{∘}as defined herein). Potentially useful humanizing substitutions can be ascertained by comparing the sequence of the framework regions of a naturally occurring VHH sequence with the corresponding framework sequence of one or more closely related human VH sequences, after which one or more of the potentially useful humanizing substitutions ^{∘}or combinations thereof) thus determined can be introduced into said VHH sequence ^{∘}in any manner known per se, as further described herein) and the resulting humanized VHH sequences can be tested for affinity for the target, for stability, for ease and level of expression, and/or for other desired properties. In this way, by means of a limited degree of trial and error, other suitable humanizing substitutions ^{∘}or suitable combinations thereof) can be determined by the skilled person. Also, based on what is described before, ^{∘}the framework regions of) an immunoglobulin single variable domain, such as a Nanobody ^{∘}including VHH domains) may be partially humanized or fully humanized. It should be noted that the immunoglobulin single variable domains, as well as the antigen-binding chimeric protein of the invention in their broadest sense are not limited to a specific biological source or to a specific method of preparation. For example but without the purpose of being limiting, the immunoglobulin single variable domains, in particular the antigen-binding chimeric proteins of the invention, can generally be obtained: ^{∘}1) by isolating the VHH domain of a naturally occurring heavy chain antibody, and further engineering of the sequence to obtain the antigen-binding chimeric protein; ^{∘}2) by expression of a nucleotide sequence encoding a naturally occurring VHH domain, in a format fused to said scaffold protein of the antigen-binding chimeric protein; ^{∘}3) by “humanization” of a naturally occurring VHH domain and/or scaffold protein or by expression of a nucleic acid encoding a such humanized VHH domain and/or scaffold protein, and/or antigen-binding chimeric protein; ^{∘}4) by “mutation” of a naturally occurring VHH domain to reduce binding to pre-existing antibodies or by engineering of the scaffold protein fusion sites to obtain an antigen-binding chimeric protein of the invention with reduced binding to pre-existing antibodies as compared to the natural VHH; or ^{∘}5) by using synthetic or semisynthetic techniques for preparing proteins, polypeptides or other amino acid sequences known per se.

For numbering of the amino acid residues of an IVD different numbering schemes can be applied. For example, numbering can be performed according to the AHo numbering scheme for all heavy ^{∘}VH) and light chain variable domains ^{∘}VL) given by Honegger, A. and Pluckthun, A. ^{∘}J. Mol. Biol. 309, 2001), as applied to VHH domains from camelids. Alternative methods for numbering the amino acid residues of VH domains, which can also be applied in an analogous manner to VHH domains, are known in the art. For example, the delineation of the FR and CDR sequences can be done by using the Kabat numbering system as applied to VHH domains from camelids in the article of Riechmann, L. and Muyldermans, S., 231^{∘}1-2), J Immunol Methods. 1999. It should be noted that—as is well known in the art for VH domains and for VHH domains—the total number of amino acid residues in each of the CDRs may vary and may not correspond to the total number of amino acid residues indicated by the Kabat numbering ^{∘}that is, one or more positions according to the Kabat numbering may not be occupied in the actual sequence, or the actual sequence may contain more amino acid residues than the number allowed for by the Kabat numbering). This means that, generally, the numbering according to Kabat may or may not correspond to the actual numbering of the amino acid residues in the actual sequence. The total number of amino acid residues in a VH domain and a VHH domain will usually be in the range of from 110 to 120, often between 112 and 115. It should however be noted that smaller and longer sequences may also be suitable for the purposes described herein.

An “epitope”, as used herein, refers to an antigenic determinant of a polypeptide, constituting a binding site or binding pocket on a target molecule. An epitope could comprise 1, 2 or 3 amino acids in a spatial conformation, which is unique to the epitope. Generally, an epitope consists of at least 4, 5, 6, 7 such amino acids, and more usually, consists of at least 8, 9, 10 such amino acids. Methods of determining the spatial conformation of amino acids are known in the art, and include, for example, X-ray crystallography and multi-dimensional nuclear magnetic resonance. A “conformational epitope”, as used herein, refers to an epitope comprising amino acids in a spatial conformation that is unique to a folded 3-dimensional conformation of a polypeptide. Generally, a conformational epitope consists of amino acids that are discontinuous in the linear sequence but that come together in the folded structure of the protein. However, a conformational epitope may also consist of a linear sequence of amino acids that adopts a conformation that is unique to a folded 3-dimensional conformation of the polypeptide ^{∘}and not present in a denatured state). In protein complexes, conformational epitopes consist of amino acids that are discontinuous in the linear sequences of one or more polypeptides that come together upon folding of the different folded polypeptides and their association in a unique quaternary structure. Similarly, conformational epitopes may here also consist of a linear sequence of amino acids of one or more polypeptides that come together and adopt a conformation that is unique to the quaternary structure. The term “conformation” or “conformational state” of a protein refers generally to the range of structures that a protein may adopt at any instant in time. One of skill in the art will recognize that determinants of conformation or conformational state include a protein's primary structure as reflected in a protein's amino acid sequence ^{∘}including modified amino acids) and the environment surrounding the protein. The conformation or conformational state of a protein also relates to structural features such as protein secondary structures ^{∘}e.g., α-helix, β-sheet, among others), tertiary structure ^{∘}e.g., the 3-dimensional folding of a polypeptide chain), and quaternary structure ^{∘}e.g., interactions of a polypeptide chain with other protein subunits). Posttranslational and other modifications to a polypeptide chain such as ligand binding, phosphorylation, sulfation, glycosylation, or attachments of hydrophobic groups, among others, can influence the conformation of a protein. Furthermore, environmental factors, such as pH, salt concentration, ionic strength, and osmolality of the surrounding solution, and interaction with other proteins and co-factors, among others, can affect protein conformation. The conformational state of a protein may be determined by either functional assay for activity or binding to another molecule or by means of physical methods such as X-ray crystallography, NMR, or spin labelling, among other methods. For a general discussion of protein conformation and conformational states, one is referred to Cantor and Schimmel, Biophysical Chemistry, Part I: The Conformation of Biological. Macromolecules, W.H. Freeman and Company, 1980, and Creighton, Proteins: Structures and Molecular Properties, W.H. Freeman and Company, 1993.

The term “affinity”, as used herein, generally refers to the degree to which an antibody or other binding protein ^{∘}as defined further herein) binds to a target protein so as to shift the equilibrium of target protein and binding protein toward the presence of a complex formed by their binding. Thus, for example, where an antibody and an antigen are combined in relatively equal concentration, an antibody of high affinity will bind to the antigen so as to shift the equilibrium toward high concentration of the resulting complex. The equilibrium dissociation constant K_{dis} ^{∘}also referred to herein as K_{D}) is commonly used to describe the affinity between a ligand and a target protein, or an antibody and its antigen. K_{dis} is the calculated ratio of k_{off}/kₒₙ, between the antibody and its antigen. The association constant ^{∘}kₒₙ) is used to characterize how quickly the antibody binds to its target. The dissociation constant ^{∘}k_{off}) is used to measure how quickly an antibody dissociates from its target and is expressed as number of units that dissociated from a target per second. Hence, the lower k_{off} is, the higher the affinity towards the target. k_{off} and thus also K_{dis} is inversely related to affinity. A high affinity interaction is characterized by a low K_{dis}, a fast recognizing ^{∘}high kₒₙ) and a strong stability of formed complexes ^{∘}low k_{off}).

It will be appreciated that within the scope of the present application, the term “affinity” is used in the context of the antibody or antibody fragment that binds an epitope of the folate receptor FRα, more particularly the antibody or antibody fragment is “functional” in binding its target via the paratope, which typically involves one or more CDRs, of its immunoglobulin ^{∘}Ig) domain.

“Amino acids” as used herein refer to the structural units ^{∘}monomers) that make up proteins. They join together to form short polymer chains called peptides or longer chains called either polypeptides or proteins. These chains are linear and unbranched, with each amino acid residue within the chain attached to two neighbouring amino acids. Twenty amino acids encoded by the universal genetic code are naturally incorporated into polypeptides and are called proteinogenic or natural amino acids. Natural amino acids or naturally occurring amino acids are glycine ^{∘}Gly or G), Alanine ^{∘}Ala or A), Valine ^{∘}Val or V), Leucine ^{∘}Leu or L), Isoleucine ^{∘}Ile or I), Methionine ^{∘}Met or M), Proline ^{∘}Pro or P), Phenylalanine ^{∘}Phe or F), Tryptophan ^{∘}Trp or W), Serine ^{∘}Ser or S), Threonine ^{∘}Thr or T), Asparagine ^{∘}Asn or N), Glutamine ^{∘}Gln or Q), Tyrosine ^{∘}Tyr or Y), Cysteine ^{∘}Cys or C), Lysine ^{∘}Lys or K), Arginine ^{∘}Arg or R), Histidine ^{∘}His or H), Aspartic Acid ^{∘}Asp or D) and Glutamic Acid ^{∘}Glu or E).

As used herein, the terms “nucleic acid”, “nucleic acid sequence” or “nucleic acid molecule” are used interchangeably and refer to a polymeric form of nucleotides of any length, either deoxyribonucleotides or ribonucleotides, or analogs thereof. Nucleic acids may have any three-dimensional structure, and may perform any function, known or unknown. Non-limiting examples of nucleic acids include a gene, a gene fragment, exons, introns, messenger RNA ^{∘}mRNA), transfer RNA, ribosomal RNA, ribozymes, cDNA, recombinant polynucleotides, branched polynucleotides, plasmids, vectors, isolated DNA of any sequence, control regions, isolated RNA of any sequence, nucleic acid probes, and primers. The nucleic acid molecule may be linear or circular. The nucleic acid may comprise a promoter, an intron, an enhancer region, a polyadenylation site, a translation initiation site, 5′ or 3′ untranslated regions, a reporter gene, a selectable marker or the like. The nucleic acid may comprise single stranded or double stranded DNA or RNA. The nucleic acid may comprise modified bases or a modified backbone. A nucleic acid that is up to about 100 nucleotides in length, is often also referred to as an oligonucleotide. “Nucleotides” as used herein refer to the building blocks of oligonucleotides and polynucleotides, and for the purposes of the present invention include both naturally occurring and non-naturally occurring nucleotides. In nature, nucleotides, such as DNA and RNA nucleotides comprise a ribose sugar moiety, a nucleobase moiety and one or more phosphate groups ^{∘}which are absent in nucleosides). A nucleotide without a phosphate group is called a “nucleoside” and is thus a compound comprising a nucleobase moiety and a sugar moiety. As used herein, “nucleobase” means a group of atoms that can be linked to a sugar moiety to create a nucleoside that is capable of incorporation into an oligonucleotide, and wherein the group of atoms is capable of bonding with a complementary naturally occurring nucleobase of another oligonucleotide or nucleic acid. Naturally occurring nucleobases of RNA or DNA comprise the purine bases adenine ^{∘}A) and guanine ^{∘}G), and the pyrimidine bases thymine ^{∘}T), cytosine ^{∘}C) and uracil ^{∘}U).

“Nucleotide sequence”, “DNA sequence” or “nucleic acid molecule^{∘}s)” as used herein refers to a polymeric form of nucleotides of any length, either ribonucleotides or deoxyribonucleotides. This term refers only to the primary structure of the molecule. Thus, this term includes double- and single-stranded DNA, the ^{∘}reverse) complement DNA, and RNA. It also includes known types of modifications, for example, methylation, “caps” substitution of one or more of the naturally occurring nucleotides with an analogue. By “nucleic acid construct” it is meant a nucleic acid sequence that has been constructed to comprise one or more functional units not found together in nature. Examples include circular, linear, double-stranded, extrachromosomal DNA molecules ^{∘}plasmids), cosmids ^{∘}plasmids containing COS sequences from lambda phage), viral genomes comprising non-native nucleic acid sequences, and the like. “Coding sequence” is a nucleotide sequence, which is transcribed into mRNA and/or translated into a polypeptide when placed under the control of appropriate regulatory sequences. The boundaries of the coding sequence are determined by a translation start codon at the 5′-terminus and a translation stop codon at the 3′-terminus. A coding sequence can include, but is not limited to mRNA, cDNA, recombinant nucleotide sequences or genomic DNA, while introns may be present as well under certain circumstances.

An “expression cassette” as used herein comprises any nucleic acid construct capable of directing the expression of a gene/coding sequence of interest, which is operably linked to a promoter of the expression cassette. Expression cassettes are generally DNA constructs preferably including ^{∘}5′ to 3′ in the direction of transcription): a promoter region, a polynucleotide sequence, homologue, variant or fragment thereof operably linked with the transcription initiation region, and a termination sequence including a stop signal for RNA polymerase and a polyadenylation signal. It is understood that all of these regions should be capable of operating in biological cells, such as prokaryotic or eukaryotic cells, to be transformed. The promoter region comprising the transcription initiation region, which preferably includes the RNA polymerase binding site, and the polyadenylation signal may be native to the biological cell to be transformed or may be derived from an alternative source, where the region is functional in the biological cell. Such cassettes can be constructed into a “vector”. The term “vector” or alternatively “vector construct”, “expression vector” or “gene transfer vector” is intended to refer to a nucleic acid molecule capable of transporting another nucleic acid molecule to which it has been linked, and includes any vector known to the skilled person, including any suitable type, but not limited to, for instance, plasmid vectors, cosmid vectors, phage vectors, such as lambda phage, viral vectors, such as adenoviral, AAV or baculoviral vectors, or artificial chromosome vectors such as bacterial artificial chromosomes ^{∘}BAC), yeast artificial chromosomes ^{∘}YAC), or P1 artificial chromosomes ^{∘}PAC). Expression vectors comprise plasmids as well as viral vectors and generally contain a desired coding sequence and appropriate DNA sequences necessary for the expression of the operably linked coding sequence in a particular host organism ^{∘}e.g., bacteria, yeast, plant, insect, or mammal) or in in vitro expression systems. Cloning vectors are generally used to engineer and amplify a certain desired DNA fragment and may lack functional sequences needed for expression of the desired DNA fragments. The construction of expression vectors for use in transfecting cells is also well known in the art, and thus can be accomplished via standard techniques ^{∘}see, for example, Sambrook, Fritsch, and Maniatis, in: Molecular Cloning, A Laboratory Manual, Cold Spring Harbor Laboratory Press, 1989; Gene Transfer and Expression Protocols, pp. 109-128, ed. E. J. Murray, The Humana Press Inc., Clif ton, N.J.), and the Ambion 1998 Catalog ^{∘}Ambion, Austin, Tex.).

The terms “identical” or percent “identity” in the context of two or more nucleic acid or amino acid sequences refer to two or more sequences that are the same or have a specified percentage of nucleotides or amino acid residues respectively that are the same, when compared and aligned ^{∘}introducing gaps, if necessary) for maximum correspondence, not considering any conservative amino acid substitutions as part of the sequence identity. The percent identity can be measured using sequence comparison software or algorithms or by visual inspection. Various algorithms and software are known in the art that can be used to obtain alignments of nucleotide or amino acid sequences.

The term “percent sequence identity” or “% sequence identity” or “percent identity” or “% identity” between two polynucleotide or polypeptide sequences refers to the number of identical matched positions shared by the sequences over a comparison window, taking into account additions or deletions ^{∘}i.e. gaps) that must be introduced for optimal alignment of the two sequences. A matched position is any position where an identical nucleotide or amino acid is presented in both the target and reference sequence. Gaps presented in the target sequence are not counted since gaps are not nucleotides or amino acids. Likewise, gaps presented in the reference sequence are not counted since target sequence nucleotides or amino acids are counted, not nucleotides or amino acids from the reference sequence.

One such non-limiting example of a sequence alignment algorithm is the algorithm described in Karlin et al., 1990, Proc. Natl. Acad. Sci., 87:2264-2268, as modified in Karlin et al., 1993, Proc. Natl. Acad. Sci., 90:5873-5877, and incorporated into the NBLAST and XBLAST programs ^{∘}Altschul et al., 1991, Nucleic Acids Res., 25:3389-3402). In certain aspects, Gapped BLAST can be used as described in Altschul et al., 1997, Nucleic Acids Res. 25:3389-3402. BLAST-2, WU-BLAST-2 ^{∘}Altschul et al., 1996, Methods in Enzymology, 266:460-480), ALIGN, ALIGN-2 ^{∘}Genentech, South San Francisco, California) or Megalign ^{∘}DNASTAR) are additional publicly available software programs that can be used to align sequences. In certain aspects, the percent identity between two nucleotide sequences is determined using the GAP program in the GCG software package ^{∘}e.g., using a NWSgapdna.CMP matrix and a gap weight of 40, 50, 60, 70, or 90 and a length weight of 1, 2, 3, 4, 5, or 6). In certain alternative aspects, the GAP program in the GCG software package, which incorporates the algorithm of Needleman and Wunsch J. Mol. Biol. ^{∘}48):444-453 ^{∘}1970)) can be used to determine the percent identity between two amino acid sequences ^{∘}e.g., using either a BLOSUM 62 matrix or a PAM250 matrix, and a gap weight of 16, 14, 12, 10, 8, 6, or 4 and a length weight of 1, 2, 3, 4, 5). Alternatively, in certain aspects, the percent identity between nucleotide or amino acid sequences is determined using the algorithm of Myers and Miller ^{∘}CABIOS, ^{∘}4:11-17 ^{∘}1989)). For example, the percent identity can be determined using the ALIGN program ^{∘}version 2.0) and using a PAM120 with residue table, a gap length penalty of 12 and a gap penalty of 4. One skilled in the art can determine appropriate parameters for maximal alignment by particular alignment software. In certain aspects, the default parameters of the alignment software are used.

One skilled in the art will appreciate that the generation of a sequence alignment for the calculation of a percent sequence identity is not limited to binary sequence-sequence comparisons exclusively driven by primary sequence data. Sequence alignments can be derived from multiple sequence alignments. One suitable program to generate multiple sequence alignments is ClustalW2, available from www.clustal.org. Another suitable program is MUSCLE, available from www.drive5.com/muscle/. ClustalW2 and MUSCLE are alternatively available, e.g., from the EBI ^{∘}European Bioinformatics Institute).

In certain aspects, the percentage identity “X” of a first nucleotide sequence to a second nucleotide sequence is calculated as 100×^{∘}Y/Z), where Y is the number of nucleotide residues scored as identical matches in the alignment of the first and second sequences as aligned by visual inspection or a particular sequence alignment program) and Z is the total number of residues in the second sequence. If the length of a first sequence is longer than the second sequence, the percent identity of the first sequence to the second sequence will be higher than the percent identity of the second sequence to the first sequence. Different regions within a single polynucleotide target sequence that align with a polynucleotide reference sequence can each have their own percent sequence identity. It is noted that the percent sequence identity value is rounded to the nearest tenth. For example, 80.11, 80.12, 80.13, and 80.14 are rounded down to 80.1, while 80.15, 80.16, 80.17, 80.18, and 80.19 are rounded up to 80.2. It also is noted that the length value will always be an integer.

According to the present application, the degree of identity, between a given reference nucleotide sequence and a nucleotide sequence which is a homologue of said given nucleotide sequence will preferably be at least 60%, 65%, 70%, 75%, 80%, 81%, 82%, 83%, 84%, 85%, 86%, 87%, 88%, 89%, 90%, 91%, 92%, 93%, 94%, 95%, 96%, 97%, 98%, or 99%. The degree of identity is given preferably for a nucleic acid region which is at least 10%, at least 20%, at least 30%, at least 40%, at least 50%, at least 60%, at least 70%, at least 80%, at least 90% or 100% of the entire length of the reference nucleic acid sequence.

For example, if the reference nucleic acid sequence consists of 200 nucleotides, the degree of identity is given preferably for at least 20, at least 40, at least 60, at least 80, at least 100, at least 120, at least 140, at least 160, at least 180, or 200 nucleotides, preferably contiguous nucleotides. In a particular embodiment, the degree/percentage of similarity or identity is given for the entire length of the reference nucleic acid sequence.

The term “amino acid identity” as used herein refers to the extent that sequences are identical on an amino acid-by-amino acid basis over a window of comparison. Thus, a “percentage of sequence identity” is calculated by comparing two optimally aligned sequences over the window of comparison, determining the number of positions at which the identical amino acid residue e.g., Ala, Pro, Ser, Thr, Gly, Val, Leu, Ile, Phe, Tyr, Trp, Lys, Arg, His, Asp, Glu, Asn, Gln, Cys and Met) occurs in both sequences to yield the number of matched positions, dividing the number of matched positions by the total number of positions in the window of comparison ^{∘}i.e., the window size), and multiplying the result by 100 to yield the percentage of sequence identity. According to the present application, the degree of identity, between a given reference amino acid sequence and an amino acid sequence which is a homologue of said given amino acid sequence will preferably be at least 60%, 65%, 70%, 75%, 80%, 81%, 82%, 83%, 84%, 85%, 86%, 87%, 88%, 89%, 90%, 91%, 92%, 93%, 94%, 95%, 96%, 97%, 98%, or 99%. The degree of identity is given preferably for an amino acid region which is at least 10%, at least 20%, at least 30%, at least 40%, at least 50%, at least 60%, at least 70%, at least 80%, at least 90% or 100% of the entire length of the reference amino acid sequence. For example, if the reference amino acid sequence consists of 200 amino acids, the degree of identity is given preferably for at least 20, at least 40, at least 60, at least 80, at least 100, at least 120, at least 140, at least 160, at least 180, or 200 amino acids, preferably contiguous amino acids. In a particular embodiment, the degree/percentage of similarity or identity is given for the entire length of the reference amino acid sequence.

“Homologue” or “homologues” of a protein encompass peptides, oligopeptides, polypeptides, proteins and enzymes having amino acid substitutions, deletions and/or insertions relative to the unmodified protein in question and having similar biological and functional activity as the unmodified protein from which they are derived.

The term “defined by SEQ ID No. X”, “as present in SEQ ID No. X”, or “as depicted in SEQ ID No. X” as used herein refers to a biological sequence consisting of the sequence of amino acids or nucleotides given in the SEQ ID No. X. For instance, a protein defined in/by SEQ ID No. X consists of the amino acid sequence given in SEQ ID No. X. A further example is an amino acid sequence comprising SEQ ID No. X, which refers to an amino acid sequence longer than the amino acid sequence given in SEQ ID No. X but entirely comprising the amino acid sequence given in SEQ ID No. X ^{∘}wherein the amino acid sequence given in SEQ ID No. X can be located N-terminally or C-terminally in the longer amino acid sequence, or can be embedded in the longer amino acid sequence), or to an amino acid sequence consisting of the amino acid sequence given in SEQ ID No. X.

The term “in vivo medical imaging” refers to the technique and process that is used to visualize the inside of an organism's body ^{∘}or parts and/or functions thereof), for clinical purposes ^{∘}e.g. disease diagnosis, prognosis or therapy monitoring) or medical science ^{∘}e.g. study of anatomy and physiology). Examples of medical imaging methods include invasive techniques, such as intravascular ultrasound ^{∘}IVUS), as well as non-invasive techniques, such as magnetic resonance imaging ^{∘}MRI), ultrasound ^{∘}US) and nuclear imaging. Examples of nuclear imaging include positron emission tomography ^{∘}PET) and single photon emission computed tomography ^{∘}SPECT). In a preferred embodiment, a nuclear imaging approach is used for in vivo medical imaging. According to one specific embodiment, in vivo pinhole SPECT/micro-CT computed tomography) imaging is used as in vivo imaging approach.

As used herein, the term “radionuclide” relates to a radioactive label, which is a chemical compound in which one or more atoms have been replaced by a radioisotope. Radionuclides vary based on their characteristics, which include half-life, energy emission characteristics, and type of decay. This allows one to select radionuclides that have the desired mixture of characteristics suitable for use diagnostically and/or therapeutically. For example, gamma emitters are generally used diagnostically and alpha and beta emitters are generally used therapeutically. However, some radionuclides are both gamma emitters, alpha emitters and/or beta emitters, and thus, may be suitable for both uses. Radionuclides, as used herein, include for example—but not limited to—Actinium-225, Astatine-209, Astatine-210, Astatine-211, Bismuth-212, Bismuth-213, Brome-76, Caesium-137, Carbon-11, Chromium-51, Cobalt-60, Copper-64, Copper-67, Dysprosium-165, Erbium-169, Fermium-255, Fluorine-18, Gallium-67, Gallium-68, Gold-198, Holium-166, Indium-111, Iodine-123, Iodine-124, Iodine-125, Iodine-131, Iridium-192, Iron-59, Krypton-81m, Lead-212, Lutetium-177, Molydenum-99, Nitrogen-13, Oxygen-15, Palladium-103, Phosphorus-32, Potassium-42, Radium-223, Rhenium-186, Rhenium-188, Samarium-153, Technetium-99m, Radium-223, Rubidium-82, Ruthenium-106, Sodium-24, Strontium-89, Terbium-149, Thallium-201, Thorium-227, Xenon-133, Ytterbium-169, Ytterbium-177, Yttrium-86, Yttrium-90, Zirconium-89. In certain embodiments, the radionuclide is selected from the group of radionuclides as described above. In a specific embodiment, the radionuclide is selected from the group consisting of Technetium-99m, Gallium-68, Fluorine-18, Indium-111, Zirconium-89, Iodine-123, Iodine-124, Iodine-131, Astatine-211, Bismuth-213, Lutetium-177 and Yttrium-86.

A “patient” or “subject”, for the purpose of this application, relates to any organism such as a vertebrate, particularly any mammal, including both a human and another mammal, e.g., an animal such as a rodent, a rabbit, a cow, a sheep, a horse, a dog, a cat, a lama, a pig, or a non-human primate ^{∘}e.g., a monkey). In one embodiment, the patient is a human, a rat or a non-human primate. Preferably, the patient is a human. In one embodiment, a patient is a subject with or suspected of having a disease or disorder, or an injury. In the context of this application, the disease is cancer, more particularly cancer characterised by FOLR1 expressing tumor cells.

The terms “treatment” or “treating” or “treat” can be used interchangeably and are defined by a therapeutic intervention that slows, interrupts, arrests, controls, stops, reduces, or reverts the progression or severity of a sign, symptom, disorder, condition, injury, or disease, but does not necessarily involve a total elimination of all disease-related signs, symptoms, conditions, or disorders. Those in need of treatment include those already diagnosed with the disorder as well as those prone or predisposed to contract the disorder or those in whom the disorder is to be prevented. For example, in tumor ^{∘}e.g. cancer) treatment, a therapeutic agent can directly decrease the pathology of tumor cells, or render the tumor cells more susceptible to treatment by other therapeutic agents or by the subject's own immune system.

As used herein, the term “therapeutically effective amount” means the amount needed to achieve the desired result or results when used in therapy.

As used herein, the terms “diagnosis”, “prognosis” and/or “prediction” comprise diagnosing, prognosing and/or predicting a certain disease and/or disorder, thereby predicting the onset and/or presence of a certain disease and/or disorder, and/or predicting the progress and/or duration of a certain disease and/or disorder, and/or predicting the response of a patient suffering from a certain disease and/or disorder to therapy.

The term “statistically significantly” different is well known by the person skilled in the art. Statistical significance plays a pivotal role in statistical hypothesis testing. It is used to determine whether the null hypothesis should be rejected or retained. The null hypothesis is the default assumption that nothing happened or changed. For the null hypothesis to be rejected, an observed result has to be statistically significant, i.e. the observed p-value is less than the pre-specified significance level a. The p-value of a result, p, is the probability of obtaining a result at least as extreme, given that the null hypothesis were true. In one embodiment, a is 0.05. In a more particular embodiment, a is 0.01. In an even more particular embodiment, a is 0.001.

### DETAILED DESCRIPTION

### Folate Receptor Alpha ^{∘}FRα)

Folates are a class of compounds encompassing both natural folates, e.g. Vitamin B9 and folic acid ^{∘}FA). They are essential for cells to generate nucleic acids and metabolic amino acids that are required for cell proliferation and division ^{∘}Kamen 1997 Semin Oncol 24; Goh and Koren 2008 J Obstet Gynaecol 28). Folate is transported across the cellular membrane in three ways. The main route of uptake is through the reduced folate carrier ^{∘}RFC), which is ubiquitously distributed and supports the uptake of dietary folate ^{∘}Matherly and Goldman 2003 Vitam Horm 66:403-456). The second route is through the proton coupled folate transporter ^{∘}PCFT), which utilizes the transmembrane proton gradient to mediate folate transport into the cells ^{∘}Zhao et al 2011 Annu Rev Nutr 31:177-201). Finally, folate can be transported by folate receptors, of which there are four glycopolypeptide members ^{∘}FRα, FRβ, FRγ and FRδ), with molecular weights ranging from 38 to 45 kDa ^{∘}Ledermann et al 2015 Ann Oncol 26:2034-2043). The alpha isoform, Folate Receptor alpha ^{∘}FRα) is a protein that in humans is encoded by the FOLR1 gene. FRα is a glycosylphosphatidylinositol anchored cell surface receptor that mediates endocytosis of the active form of folate ^{∘}5-methyltetrahydrofolate or 5-MTF) in a clathrin-independent manner ^{∘}Salazar and Ratnam 2007 Cancer Metastasis Rev 26:141-152; Kelemen 2006 Int J Cancer 119:243-250). Throughout current application, “folate receptor” or “folate receptor α” or “FoIR” or “FOLR” or “FR” or “FOLR1” or “FR_alpha” or “FRα” are used interchangeably and refer to the human folate receptor α as described above and depicted in SEQ ID No. 1, unless specified otherwise.

SEQ ID NO: 1: human folate receptor alpha °hFOLRα
or hFRα as used interchangeably herein)
MAQRMTTQLLLLLVWVAVVGEAQTRIAWARTELLNVCMNAKHHKEKPGP
EDKLHEQCRPWRKNACCSTNTSQEAHKDVSYLYRFNWNHCGEMAPACKR
HFIQDTCLYECSPNLGPWIQQVDQSWRKERVLNVPLCKEDCEQWWEDCR
TSYTCKSNWHKGWNWTSGFNKCAVGAACQPFHFYFPTPTVLCNEIWTHS
YKVSNYSRGSGRCIQMWFDPAQGNPNEEVARFYAAAMSGAGPWAAWPFL
LSLALMLLWLLS

The current application provides antibodies and antibody fragments that bind the human folate receptor α, more specifically a specific epitope is targeted on FRα, resulting in BCSFB crossing for binders with a specific affinity and conformation in their binding to the receptor. The development of antibodies against the human FRα is part of a promising strategy for targeted treatment and immunotherapy. Indeed, as a sufficient intake of folate is needed in rapidly proliferating cells for the one-carbon metabolic reaction and DNA biosynthesis, repair and methylation, FRα is highly expressed in several solid tumors such as ovarian, breast and lung cancers ^{∘}Cheung et al 2016 Oncotarget 7:52553-52574).

### FRα and Cancer

Approximately one third of human cancers overexpress the folate receptor ^{∘}Paulos, 2004). Various quantitative and semi-quantitative methods have been employed to measure FRα expression in tumor biopsies of patients that could potentially benefit from FRα-targeted therapies ^{∘}Parker, 2005). These methods include: methods using anti-FRα antibodies ^{∘}e.g. IHC, radioimmunoassays, quantitative autoradiography and cytofluorometric analysis), RT-PCR, FISH and radioligand binding assays ^{∘}Parker, 2005). These approaches demonstrated FRα overexpression in ovarian, kidney, lung, brain, endometrial, colorectal, pancreatic, gastric, prostate and breast cancers ^{∘}Parker et al 2005 Anal Biochem 338). Overexpression of FRα in malignant cells confers these cells a growth advantage in low folate media. Indeed, FRα upregulation in tumor tissue correlates with an elevated uptake of folate, a key nutrient for dividing cells ^{∘}Farran, 2019). FRα also seems to have a role in cellular migration and invasion and FRα overexpression is associated with tumor progression in preclinical models ^{∘}Scaranti, 2020). Moreover, FRα might mediate cell division, anchorage-independent growth and adhesive properties of cancer cells ^{∘}Scaranti, 2020).

FRα is thus an attractive and valuable anticancer drug target because of its overexpression in a range of solid epithelial tumors ^{∘}Scaranti, 2020; Meric-Bernstam and Mills 2012 Nat Rev Clin Oncol 9: 542-548). Additionally, FRα has a minimal physiological role in non-malignant tissues after embryogenesis and FRα overexpression in tumors indicates a poor prognosis for the patient ^{∘}Hartmann, 2007). Moreover, FRα has a high level of affinity for non-physiological substrates ^{∘}e.g. folic acid) and possesses immunogenicity ^{∘}Farran, 2019). RFC and PCFT are currently not direct targets of anticancer drugs ^{∘}Scaranti, 2020). The current research into FRα in cancer focusses on three main aspects, ^{∘}1) targeted anticancer therapy, ^{∘}2) tumor imaging ^{∘}enable more precise cancer surgery) and ^{∘}3) predictive biomarkers ^{∘}diagnostic marker) ^{∘}Scaranti, 2020). Exploiting FRα as a diagnostic and therapeutic target offers numerous advantages ^{∘}Popovici, 2020). One aspect is the location of FRα ^{∘}on non-malignant epithelium which express the protein at much lower to negligible levels ^{∘}Parker, 2005), which makes it inaccessible to the circulation ^{∘}Popovici, 2020). Secondly, FRα binds folic acid which is a small molecule that can rapidly penetrate solid tumors and thirdly, internalized FRα will take along folic acid conjugates within the cell, after which it will be rapidly recycled to the cell surface ^{∘}Popovici, 2020).

### FRα and CNS Transport

In normal tissues, the distribution of the folate receptor alpha is low and restricted. Interestingly, of the few specialized epithelia that do have FRα on their surface, the choroid plexus epithelial ^{∘}CPE) cells display the highest level of expression. FRα, which is hypothesized to provide the major route for the blood-CSF transport of folate, can be detected both at the apical and basolateral membranes of the CPE cells ^{∘}Grapp et al 2013 Nat Comm). It was furthermore shown that folate delivery across the BCSFB occurs via exosome-mediated delivery: folate is taken up at the basolateral membrane by FRα-mediated endocytosis, is transported to intraluminal vesicles within multivesicular bodies and is finally released into the CSF in exosomal vesicles ^{∘}Grapp et al 2013 Nat Comm). Given that the CPE cells form the BCSFB, FRα is a potential target for transcytosis-mediated delivery of cargo in the CSF from where it can homogenously spread in the brain. Hence, the FRα binding agents disclosed herein are particularly useful in diagnostic and/or therapeutic approaches wherein imaging compounds or medicaments should be delivered to or in cancer tissues or in the brain, more particular in the CSF.

### FRα Binding Agents

In a first aspect, the present application discloses binding agents, more particularly binding agents comprising antibodies, even more particularly comprising single variable domain antibodies, most particularly comprising VHHs, that recognize and bind to the mouse and/or human folate receptor alpha. These antibodies are thus in itself FRα binding agents. In various embodiments, said FRα binding agents bind to, but do not functionally modulate FRα. In other embodiments, said FRα binding agents are also able to detach from the FRα after binding to it. This is especially useful in the process of folate receptor mediated transcytosis, a process during which the folate receptor binds cargo at the basolateral side of for example the choroid plexus epithelial ^{∘}CPE) cells, transports the cargo through said cells and sets the cargo free at the apical side of the CPE cells. The FRα binding agents of current application are thus extremely helpful in brain delivery of drugs which are directly or indirectly administered in peripheral blood. The FRα binding agents of current application are equally useful in delivery of therapeutic and/or imaging compounds to cancer cells. Therefore, the present application also provides compositions comprising FRα binding agents ^{∘}see later). Said compositions can be pharmaceutical and/or imaging compositions and current application envisages their use in the treatment and/or study of various CNS diseases and/or FRα expressing cancers.

In various embodiments, the FRα binding agents of the application comprise a targeting moiety having an antigen recognition domain that recognizes an epitope present on FRα. In an embodiment, the antigen-recognition domain recognizes one or more linear epitopes present on FRα. As used herein, a linear epitope refers to any continuous sequence of amino acids present on FRα. In another embodiment, the antigen-recognition domain recognizes one or more conformational epitopes present on FRα. As used herein, a conformation epitope refers to one or more sections of amino acids ^{∘}which may be discontinuous) which form a three-dimensional surface with features and/or shapes and/or tertiary structures capable of being recognized by an antigen recognition domain.

In an embodiment, the FRα binding agent of the application comprises a targeting moiety with an antigen recognition domain that recognizes one or more epitopes present on the human FRα. In an embodiment, the human FRα comprises the amino acid sequence of SEQ ID No. 1. In a more particular embodiment, the human FRα consist of the amino acid sequence of SEQ ID No. 1. In an even more particular embodiment the FRα binding agents of the application do not compete with folic acid and thus do not bind to or interfere with the folic acid binding site of the human FRα. In another embodiment, the FRα binding agents of the application bind to the a competing or the same epitope on human FRα as 2HFO42 or alternatively phrased as the FRα binding agent comprising or consisting of the amino acid sequence as set forth in SEQ ID No. 2. In a particular embodiment, the FRα binding agent of the application binds to a conformational epitope present on FRα, wherein said epitope comprises residue Q141 of SEQ ID No. 1, or more particularly comprises at least two or more residues selected from R98, H99, E137, D138, Q141, E144, D145, R204, G205, Q211, W213, F214, D215, and A217 of SEQ ID No. 1. This means that amino acid R on position 98 of SEQ ID No. 1, amino acid H on position 99, amino acid E on position 137, amino acid D on position 138, amino acid Q on position 141, amino acid E on position 144, amino acid D on position 145, amino acid R on position 204, amino acid G on position 205, amino acid Q on position 211, amino acid W on position 213, amino acid F on position 214, amino acid D on position 215, and amino acid A on position 217, are part of the conformational epitope. In another particular embodiment, the FRα binding agent of the application binds to a conformational epitope present on FRα, wherein said epitope comprises or consists of R98, H99, E137, D138, Q141, E144, D145, R204, G205, Q211, W213, F214, D215, and A217 of SEQ ID No. 1.

In one embodiment, the FRα binding agent of the application comprises a full-length multimeric protein that includes two heavy chains and two light chains. Each heavy chain includes one variable region ^{∘}e.g. VH) and at least three constant regions ^{∘}e.g. CH1, CH2 and CH3), and each light chain includes one variable region ^{∘}VL) and one constant region ^{∘}CL). As described above in the definitions section, the variable regions determine the specificity of the antibody and comprise three hypervariable regions also known as complementarity determining regions ^{∘}CDRs) that contribute to the antibody binding specificity.

In some embodiments, the FRα binding agent comprises a targeting moiety which is an antibody fragment. The term “antibody fragment” refers to a portion of any antibody or antibody-like structure that by itself has high affinity for an antigenic determinant or epitope, and contains one or more CDRs accounting for such specificity. In some particular embodiments, the FRα binding agent of the application comprises a targeting moiety which is a single-domain antibody, an immunoglobulin single variable domain, a heavy-chain-only antibody ^{∘}VHH), a single-chain antibody ^{∘}scFv), a shark heavy-chain-only antibody ^{∘}VNAR), a microprotein ^{∘}cysteine knot protein, knottin), a DARPin, a Tetranectin, an Affibody, an Affimer, a Transbody, an Anticalin, an AdNectin, an Affilin, a Microbody, a peptide aptamer, an alterases, a plastic antibodies, a phylomer, a stradobodies, a maxibodies, an evibody, a fynomer, an armadillo repeat protein, a Kunitz domain, an avimer, an atrimer, a probody, an immunobody, a triomab, a troybody, a pepbody, a vaccibody, a UniBody, a DuoBody, a Fv, a Fab, a Fab′, a F^{∘}ab′)2, a peptide mimetic molecule, or a synthetic molecule, as described in US Patent Nos. or Patent Publication Nos. U.S. Pat. No. 7,417,130, US 2004/132094, U.S. Pat. No. 5,831,012, US 2004/023334, U.S. Pat. Nos. 7,250,297, 6,818,418, US 2004/209243, U.S. Pat. Nos. 7,838,629, 7,186,524, 6,004,746, 5,475,096, US 2004/146938, US 2004/157209, U.S. Pat. Nos. 6,994,982, 6,794,144, US 2010/239633, U.S. Pat. No. 7,803,907, US 2010/119446, and/or U.S. Pat. No. 7,166,697, the contents of which are hereby incorporated by reference in their entireties. See also Storz 2011 MAbs 3: 310-317.

In particular embodiments, the FRα binding agent of the application comprises a targeting moiety which is a single-domain antibody, such as a VHH. The VHH may be derived from, for example, an organism that produces VHH antibodies such as a camelid, a shark, or the VHH may be a designed VHH. VHHs are antibody-derived therapeutic proteins that contain the unique structural and functional properties of naturally occurring heavy-chain antibodies ^{∘}see definition section above). In some embodiments, the single domain antibody as described herein is an immunoglobulin single variable domain or ISVD. In most particular embodiments, the FRα binding agent comprises a targeting moiety which is a VHH.

In a specific embodiment, said FRα binding agent more particularly the binding agent comprising the ISVD or VHH of the application comprises a CDR3 having an amino acid sequence with maximally two amino acids different to SEQ ID No. 5 or with maximally one amino acid different to SEQ ID No. 5 or comprises a CDR3 comprising or consisting of the amino acid sequence depicted in SEQ ID No. 5. Said CDR3 sequence represents an essential feature of a family of ISVDs, more particularly VHHs, specifically binding FRα at the same binding site.

VHHs or Nbs are often classified in different sequences families or even superfamilies, as to cluster the clonally related sequences derived from the same progenitor during B cell maturation ^{∘}Deschaght et al. 2017. Front Immunol. 10; 8:420). This classification is often based on the CDR sequence of the Nbs, and wherein for instance each Nb family is defined as a cluster of ^{∘}clonally) related sequences with a sequence identity threshold of the CDR3 region. Within a single VHH family defined herein, the CDR3 sequence is thus identical or very similar in amino acid composition, preferably with at least 80% identity, or at least 85% identity, or at least 90% identity in the CDR3 sequence, resulting in Nbs of the same family binding to the same binding site, having the same effect or functional impact.

An ISVD family is thus defined herein as a group of ISVD amino acid sequences with high similarity, or even identical, in the CDR3 sequence. By default, ISVDs belong to the same family when binding to the same target epitope. Variations in an ISVD family may be interesting if expression/stability/affinity/crystallization of a representative of that family is poor, as small deviations like single amino acid mutations occurring within one family may improve these properties. One embodiment relates to the ISVDs of the application comprising SEQ ID No. 5, or homologues thereof with at least 80%, 85%, 90%, 92%, 95%, 96%, 97%, 98% or 99% homology on amino acid level, or a humanized variant thereof.

In another specific embodiment, the FRα binding agent more particularly the agent comprising the ISVD or VHH of the application comprises a CDR3 having an amino acid sequence with maximally two amino acids different to SEQ ID No. 11, 14, 21, 26 or 30 or with maximally one amino acid different to SEQ ID No. 11, 14, 21, 26 or 30 or comprises a CDR3 comprising or consisting of the amino acid sequence depicted in SEQ ID No. 11, 14, 21, 26 or 30. In another specific embodiment, the FRα binding agent more particularly the ISVD or VHH of the application comprises a CDR3 sequence with at least 80%, 85%, 90%, 92%, 95%, 96%, 97%, 98% or 99% homology on amino acid level of SEQ ID No. 5, 11, 14, 21, 26 or 30, or a humanized variant thereof.

Table 1 provides an overview of full length and CDR sequences of the herein disclosed anti-FRα VHHs. VHH 2HFO42 and VHH 2HFO9 belong to family 3 and have a CDR1 with consensus or conserved sequence SEQ ID No.113: X1SX2FX3GMX4MG wherein X1 is G or E, X2 G, T, or P, X3 S or I and X4 I or L; with CDR2 with conserved sequence SEQ ID No. 31: TX1TSHGTTNYADSVKG, wherein X1 is V or I, or with conserved sequence SEQ ID No. 32: TX1TSX2GTTNYADSVKG, wherein X1 is V or I and X2 is H or G. Further embodiments related to the anti-FRα VHHs wherein the sequence of the FRs are defined as FR1 having a consensus or conserved sequence depicted as SEQ ID No. 114: X1VQLX2ESGGGLVQX3GGSLRLSCAAS wherein X1 is Q, E, D, X2 is Q or V, and X3 is A or P; FR2 having a conserved sequence depicted as SEQ ID No.115: WYRQX1PGKQRELVA, wherein X1 is V or A, FR3 having a conserved sequence depicted as SEQ ID No.116: RFTISRX1X2AKNTVX3LQMNSLX4PEDTAVYYC wherein X1 is D, E, or P, X2 is N or G, X3 is L or Y, X4 is K or R; and FR4 having a conserved sequence depicted as SEQ ID No. 117: WGXIGTX2VTVSS wherein X1 is K or Q, and X2 is Q or L.

TABLE 1
Overview of full length and CDR sequences of obtained FRa binding VHHs

| Family | VHH | Full length | CDR1 | CDR2 | CDR3 |
|---|---|---|---|---|---|
| 3 | 2HFO42 | SEQ ID No. 2 | SEQ ID No. 3 | SEQ ID No. 4 | SEQ ID No. 5 |
| | | | GSGFSGMIMG | TVTSHGTTNYADSVKG | NAHFPGIY |
| | 2HFO9 | SEQ ID No. 6 | SEQ ID No. 3 | SEQ ID No. 7 | SEQ ID No. 5 |
| | | | GSGFSGMIMG | TITSGGTTNYADSVKG | NAHFPGIY |
| | 2MFR67 | SEQ ID No. | SEQ ID No. 3 | SEQ ID No. 4 | SEQ ID No. 5 |
| | | 36 | GSGFSGMIMG | TVTSHGTTNYADSVKG | NAHFPGIY |
| 1 | 2HFO19 | SEQ ID No. 8 | SEQ ID No. 9 | SEQ ID No. 10 | SEQ ID No. 11 |
| | | | GFPFSTVYMS | GINNDGGVIDYADSVKG | ARGRSFVATLSSSLR |
| | 3MFR73 | SEQ ID No. | SEQ ID No. 9 | SEQ ID No. 13 | SEQ ID No. 14 |
| | | 12 | GFPFSTVYMS | GINSNGEIIDYADSVKG | ARGRAFVATLPSSLR |
| | 2MFR84 | SEQ ID No. | SEQ ID No. 16 | SEQ ID No. 17 | SEQ ID No. 14 |
| | | 15 | GFPFSTYYMS | GINSDGGVIDYADSVKG | ARGRAFVATLPSSLR |
| | 2MFR63 | SEQ ID No. | SEQ ID No. 16 | SEQ ID No. 19 | SEQ ID No. 14 |
| | | 18 | GFPFSTYYMS | GINSNGGVIDYADSVKG | ARGRAFVATLPSSLR |
| | 3HFO26 | SEQ ID No. | SEQ ID No.16 | SEQ ID No. 19 | SEQ ID No. 21 |
| | | 20 | GFPFSTYYMS | GINSNGGVIDYADSVKG | ARGRAFVATMPSSLR |
| | 2MFRO7 | SEQ ID No. | SEQ ID No. 16 | SEQ ID No. 19 | SEQ ID No. 21 |
| | | 22 | GFPFSTYYMS | GINSNGGVIDYADSVKG | ARGRAFVATMPSSLR |
| 7 | 3HFO28 | SEQ ID No. | SEQ ID No. 24 | SEQ ID No. 25 | SEQ ID No. 26 |
| | | 23 | GSTLDRYEIG | CISTTSSTYEDSVKGRFTIS | ATVRVAWSTCFPTGDS |
| 10 | 3MFR60 | SEQ ID No. | SEQ ID No. 28 | SEQ ID No. 29 | SEQ ID No. 30 |
| | | 27 | GIIFSMNGMG | SITSGGSTKYADSVKG | NIQFGSRWSTYW |

VHHs 2HFO19, 3MFR73, 2MFR84, 2MFR63, 3HFO26 and 2MFRO7 belong to family 1 and have a CDR1 with conserved sequence SEQ ID No. 33: GFPFSTX1YMS, wherein X1 is V or Y, a CDR2 with conserved sequence SEQ ID No. 34: GINX1X2GX3X4IDYADSVKG, wherein X1 is N or 5, X2 is D or N, X3 is G or E and X4 is V or I and a CDR3 with conserved sequence SEQ ID No. 35: ARGRX1FVATX2X3SSLR, wherein X1 is S or A, X2 is L or M and X3 is S or P.

In some embodiments, the FRα binding agent of the application comprises a targeting moiety which is a VHH comprising a single amino acid chain having four “framework regions” and three “complementary determining regions” or CDRs. As used herein, “framework region” refers to a region in the variable domain which is located between the CDRs. As used herein, “complementary determining region” or “CDR” refers to variable loop regions in VHHs that contains the amino acid sequences capable of specifically binding to antigenic targets, so potentially forming the paratope.

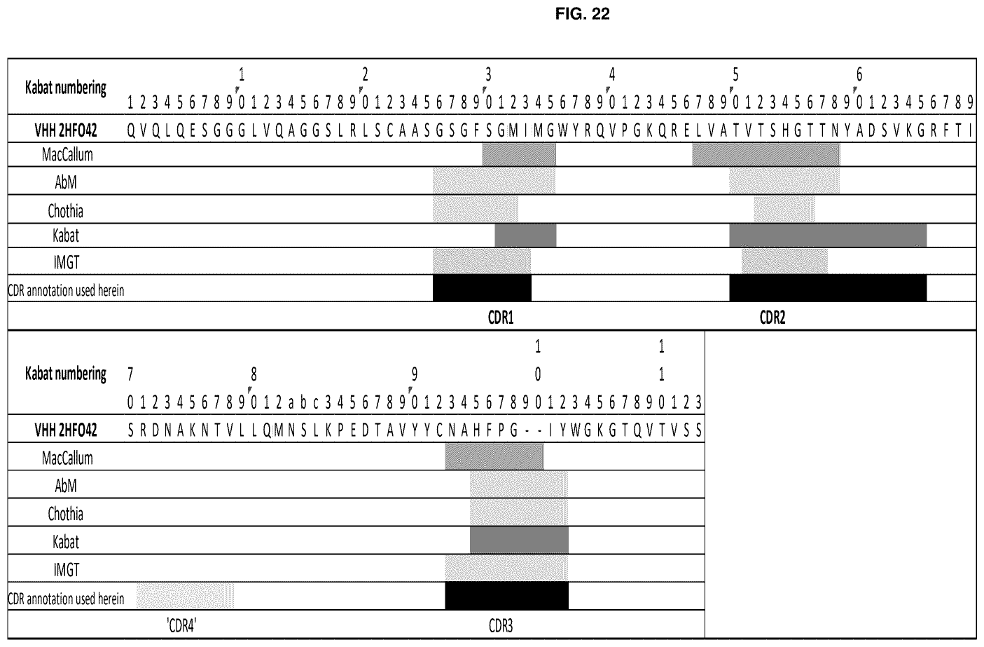

So in a specific embodiment, the FRα binding agent comprises an ISVD which binds the receptor via the residues of the ISVD of family 3 as described herein, at positions 29, 30, 31 and 33 of CDR1, 52, 53, 54 and 56 of CDR2, and 95, 96, 97, 98, 101 and 102 of CDR3, wherein Kabat numbering is used to define the amino acid positions of the ISVD, as illustrated for 2HFO42 SEQ ID No.2 in FIG. 22. In a further embodiment, as described in the examples, the ‘CDR4’ or DE loop region located in FR3 impacting the properties of the ISVD, specifically the ability to cross the BCSFB, providing for ISVDs further limited to a CDR4 sequence wherein position 72 is a D, position 73 is an N, or alternatively position 72 is an E and position 73 is a G, or alternatively, position 72 is a P and position 73 is a G, according to Kabat numbering as referred to in SEQ ID No.2. More specifically said CDR4 may be restricted to amino acids R at position 71, D, E or P at position 72, N or G at position 73, A at position 74, K at position 75, N at position 76, and T at position 77, according to Kabat numbering ^{∘}for instance see FIG. 22).

In various embodiments, the FRα binding agent comprises a VHH having a variable domain comprising at least one CDR1, CDR2, and/or CDR3 sequence. In some embodiments, the CDR sequence of the ISVD of the FRα binding agent is defined by the CDRs from Seq ID No.2 as annotated according to Chothia, AbM, Maccallum, IMGT or Kabat annotations, as known in the art and as described and illustrated herein ^{∘}FIG. 22).

In a further embodiment, the CDR1 sequence is selected from SEQ ID No. 3, 9, 16, 24 or 28. In some embodiments, the CDR2 sequence is selected from SEQ ID No. 4, 7, 10, 13, 17, 19, 25, 29 or 31. In some embodiments, the CDR3 sequence is selected from SEQ ID No. 5, 11, 14, 21, 26 or 30.

In more specific embodiments, the binding agents as described herein relate to anti-FRα VHHs with a sequence that is a humanized variant or affinity variant or sequence optimized variant as described and exemplified herein, and as provided in the sequence listing, wherein said amino acid sequences are not limited to the tagged or fused versions, but only to their CDR and FR sequences, as provided in the format of an ISVD being FR1-CDR1-FR2-CDR2-FR3-‘CDR4’-FR3-CDR3-FR4.

In a particular embodiment, a FRα binding agent is provided, said agent has an amino acid sequence of at least 80%, at least 85%, at least 90%, at least 91%, at least 92%, at least 93%, at least 94%, at least 95%, at least 96%, at least 97%, at least 98%, at least 99% homology to SEQ ID No. 2 or 37, said agent comprising three complementarity determining regions ^{∘}CDR1, CDR2 and CDR3), wherein CDR1 comprises or consist of SEQ ID No. 3, CDR2 comprises or consist of SEQ ID No. 4 and CDR3 comprises or consist of SEQ ID No. 5. In particular embodiments, said differences in amino acid sequence between said homologues and SEQ ID No. 2 are found in the framework regions. The role of framework regions in specific binding to the target is rather limited and variations in the framework sequences are allowed to obtain a similar efficacy of said ISVDs ^{∘}see for instance De Groeve et al 2010 J Nuclear Medicine 51:782; Saerens et al 2005 J Mol Biol 352:597-607). In particular embodiments, said differences in amino acid sequence have been introduced for example for humanization purposes ^{∘}see below). In even more particular embodiments, said differences in amino acid sequences are limited to conserved amino acid substitutions ^{∘}see below). In most particular embodiments, a FRα binding agent is provided wherein said FRα binding agent is represented by SEQ ID No. 2 or 37.

In a particular embodiment, a FRα binding agent is provided, said agent has an amino acid sequence of at least 80%, at least 85%, at least 90%, at least 91%, at least 92%, at least 93%, at least 94%, at least 95%, at least 96%, at least 97%, at least 98%, at least 99% homology to SEQ ID No. 6, said agent comprising three complementarity determining regions ^{∘}CDR1, CDR2 and CDR3), wherein CDR1 comprises or consist of SEQ ID No. 3, CDR2 comprises or consist of SEQ ID No. 7 and CDR3 comprises or consist of SEQ ID No. 5. In particular embodiments, said differences in amino acid sequence between said homologues and SEQ ID No. 6 are found in the framework regions. In most particular embodiments, a FRα binding agent is provided wherein said FRα binding agent is represented by SEQ ID No. 6.

In a particular embodiment, a FRα binding agent is provided, said agent has an amino acid sequence of at least 80%, at least 85%, at least 90%, at least 91%, at least 92%, at least 93%, at least 94%, at least 95%, at least 96%, at least 97%, at least 98%, at least 99% homology to SEQ ID No. 36, said agent comprising three complementarity determining regions ^{∘}CDR1, CDR2 and CDR3), wherein CDR1 comprises or consist of SEQ ID No. 3, CDR2 comprises or consist of SEQ ID No. 4 and CDR3 comprises or consist of SEQ ID No. 5. In particular embodiments, said differences in amino acid sequence between said homologues and SEQ ID No. 36 are found in the framework regions. In most particular embodiments, a FRα binding agent is provided wherein said FRα binding agent is represented by SEQ ID No. 36.

In a particular embodiment, a FRα binding agent is provided, said agent has an amino acid sequence of at least 80%, at least 85%, at least 90%, at least 91%, at least 92%, at least 93%, at least 94%, at least 95%, at least 96%, at least 97%, at least 98%, at least 99% homology to SEQ ID No. 12, said agent comprising three complementarity determining regions ^{∘}CDR1, CDR2 and CDR3), wherein CDR1 comprises or consist of SEQ ID No. 9, CDR2 comprises or consist of SEQ ID No. 13 and CDR3 comprises or consist of SEQ ID No. 14. In particular embodiments, said differences in amino acid sequence between said homologues and SEQ ID No. 12 are found in the framework regions. In most particular embodiments, a FRα binding agent is provided wherein said FRα binding agent is represented by SEQ ID No. 12.

In a particular embodiment, a FRα binding agent is provided, said agent has an amino acid sequence of at least 80%, at least 85%, at least 90%, at least 91%, at least 92%, at least 93%, at least 94%, at least 95%, at least 96%, at least 97%, at least 98%, at least 99% homology to SEQ ID No. 15, said agent comprising three complementarity determining regions ^{∘}CDR1, CDR2 and CDR3), wherein CDR1 comprises or consist of SEQ ID No. 16, CDR2 comprises or consist of SEQ ID No. 17 and CDR3 comprises or consist of SEQ ID No. 14. In particular embodiments, said differences in amino acid sequence between said homologues and SEQ ID No. 15 are found in the framework regions. In most particular embodiments, a FRα binding agent is provided wherein said FRα binding agent is represented by SEQ ID No. 15.

In a particular embodiment, a FRα binding agent is provided, said agent has an amino acid sequence of at least 80%, at least 85%, at least 90%, at least 91%, at least 92%, at least 93%, at least 94%, at least 95%, at least 96%, at least 97%, at least 98%, at least 99% homology to SEQ ID No. 18, said agent comprising three complementarity determining regions ^{∘}CDR1, CDR2 and CDR3), wherein CDR1 comprises or consist of SEQ ID No. 16, CDR2 comprises or consist of SEQ ID No. 19 and CDR3 comprises or consist of SEQ ID No. 14. In particular embodiments, said differences in amino acid sequence between said homologues and SEQ ID No. 18 are found in the framework regions. In most particular embodiments, a FRα binding agent is provided wherein said FRα binding agent is represented by SEQ ID No. 18.

In a particular embodiment, a FRα binding agent is provided, said agent has an amino acid sequence of at least 80%, at least 85%, at least 90%, at least 91%, at least 92%, at least 93%, at least 94%, at least 95%, at least 96%, at least 97%, at least 98%, at least 99% homology to SEQ ID No. 20, said agent comprising three complementarity determining regions ^{∘}CDR1, CDR2 and CDR3), wherein CDR1 comprises or consist of SEQ ID No. 16, CDR2 comprises or consist of SEQ ID No. 19 and CDR3 comprises or consist of SEQ ID No. 21. In particular embodiments, said differences in amino acid sequence between said homologues and SEQ ID No. 20 are found in the framework regions. In most particular embodiments, a FRα binding agent is provided wherein said FRα binding agent is represented by SEQ ID No. 20.

In a particular embodiment, a FRα binding agent is provided, said agent has an amino acid sequence of at least 80%, at least 85%, at least 90%, at least 91%, at least 92%, at least 93%, at least 94%, at least 95%, at least 96%, at least 97%, at least 98%, at least 99% homology to SEQ ID No. 22, said agent comprising three complementarity determining regions ^{∘}CDR1, CDR2 and CDR3), wherein CDR1 comprises or consist of SEQ ID No. 16, CDR2 comprises or consist of SEQ ID No. 19 and CDR3 comprises or consist of SEQ ID No. 21. In particular embodiments, said differences in amino acid sequence between said homologues and SEQ ID No. 22 are found in the framework regions. In most particular embodiments, a FRα binding agent is provided wherein said FRα binding agent is represented by SEQ ID No. 22.

In a particular embodiment, a FRα binding agent is provided, said agent has an amino acid sequence of at least 80%, at least 85%, at least 90%, at least 91%, at least 92%, at least 93%, at least 94%, at least 95%, at least 96%, at least 97%, at least 98%, at least 99% homology to SEQ ID No. 23, said agent comprising three complementarity determining regions ^{∘}CDR1, CDR2 and CDR3), wherein CDR1 comprises or consist of SEQ ID No. 24, CDR2 comprises or consist of SEQ ID No. 25 and CDR3 comprises or consist of SEQ ID No. 26. In particular embodiments, said differences in amino acid sequence between said homologues and SEQ ID No. 23 are found in the framework regions. In most particular embodiments, a FRα binding agent is provided wherein said FRα binding agent is represented by SEQ ID No. 23.

In a particular embodiment, a FRα binding agent is provided, said agent has an amino acid sequence of at least 80%, at least 85%, at least 90%, at least 91%, at least 92%, at least 93%, at least 94%, at least 95%, at least 96%, at least 97%, at least 98%, at least 99% homology to SEQ ID No. 27, said agent comprising three complementarity determining regions ^{∘}CDR1, CDR2 and CDR3), wherein CDR1 comprises or consist of SEQ ID No. 28, CDR2 comprises or consist of SEQ ID No. 29 and CDR3 comprises or consist of SEQ ID No. 30. In particular embodiments, said differences in amino acid sequence between said homologues and SEQ ID No. 27 are found in the framework regions. In most particular embodiments, a FRα binding agent is provided wherein said FRα binding agent is represented by SEQ ID No. 27.

### Humanization

In one embodiment, the FRα binding agent of current application comprises an immunoglobulin single variable domain or a VHH that has been “humanized”, i.e. one or more amino acid residues in the amino acid sequence of the VHH obtained by immunization is replaced by one or more of the amino acid residues that occur at the corresponding position^{∘}s) in a VH domain from a conventional 4-chain antibody from a human being to increase the degree of sequence identity with the closest human germline sequence. Potentially useful humanizing substitutions can be ascertained by comparing the sequence of the framework regions of a naturally occurring VHH sequence with the corresponding framework sequence of one or more closely related human VH sequence^{∘}s), after which one or more of the potentially useful humanizing substitutions ^{∘}or combinations thereof) thus determined can be introduced into said VHH sequence ^{∘}in any manner known per se, as further described herein) and the resulting humanized VHH sequences can be tested for affinity for the target, for stability, for ease and level of expression, and/or for other desired properties. In this way, by means of a limited degree of trial and error, other suitable humanizing substitutions ^{∘}or suitable combinations thereof) can be determined by the skilled person. Also, based on what is described before, ^{∘}the framework regions of) an immunoglobulin single variable domain, such as a VHH domain may be partially humanized or fully humanized.

Therefore, in various embodiments, the FRα binding agents of the application comprise a targeting moiety comprising an amino acid sequence having one or more amino acid mutations with respect to SEQ ID No. 2. In various embodiments, the FRα binding agent comprises a targeting moiety comprising an amino acid sequence having one, or two, or three, or four, or five, or six, or seven, or eight, or nine, or ten, or fifteen, or twenty amino acid mutations with respect to SEQ ID No. 2. In some embodiments, the one or more amino acid mutations may be independently selected from substitutions, insertions, deletions, and truncations. In some embodiments, the amino acid mutations are amino acid substitutions, and may include conservative and/or non-conservative substitutions. In particular embodiments, the one or more amino acid mutations may be in the CDRs of the targeting moiety ^{∘}e.g., the CDR1, CDR2 or CDR3 regions). In other particular embodiments, the one or more amino acid mutations may be in the framework regions of the targeting moiety ^{∘}e.g., the FR1, FR2, FR3, or FR4 regions). In most particular embodiments, said one or more amino acid mutations are only present in the framework regions of said FRα binding agents.

“Conservative substitutions” may be made, for instance, on the basis of similarity in polarity, charge, size, solubility, hydrophobicity, hydrophilicity, and/or the amphipathic nature of the amino acid residues involved. The 20 naturally occurring amino acids can be grouped into the following six standard amino acid groups: ^{∘}1) hydrophobic: Met, Ala, Val, Leu, Ile; ^{∘}2) neutral hydrophilic: Cys, Ser, Thr, Asn, Gln; ^{∘}3) acidic: Asp, Glu; ^{∘}4) basic: His, Lys, Arg; ^{∘}5) residues that influence chain orientation: Gly, Pro; and ^{∘}6) aromatic: Trp, Tyr, Phe. As used herein, “conservative substitutions” are defined as exchanges of an amino acid by another amino acid listed within the same group of the six standard amino acid groups shown above. For example, the exchange of Asp by Glu retains one negative charge in the so modified polypeptide. In addition, glycine and proline may be substituted for one another based on their ability to disrupt α-helices.

As used herein, “non-conservative substitutions” are defined as exchanges of an amino acid by another amino acid listed in a different group of the six standard amino acid groups ^{∘}1) to ^{∘}6) shown above.

In various embodiments, the substitutions may also include non-classical amino acids ^{∘}e.g. selenocysteine, pyrrolysine, N-formylmethionine β-alanine, GABA and δ-Aminolevulinic acid, 4-aminobenzoic acid ^{∘}PABA), D-isomers of the common amino acids, 2,4-diaminobutyric acid, α-amino isobutyric acid, 4-aminobutyric acid, Abu, 2-amino butyric acid, γ-Abu, ε-Ahx, 6-amino hexanoic acid, Aib, 2-amino isobutyric acid, 3-amino propionic acid, ornithine, norleucine, norvaline, hydroxyproline, sarcosme, citrulline, homocitrulline, cysteic acid, t-butylglycine, t-butylalanine, phenylglycine, cyclohexylalanine, β-alanine, fluoro-amino acids, designer amino acids such as β methyl amino acids, C α-methyl amino acids, N α-methyl amino acids, and amino acid analogs in general).

In most particular embodiments, said mutations introduced in VHH_2HFO42 for humanization purposes are deletion of residue M32 and/or substitution of V to A on position 40 of SEQ ID No. 2.

In other most particular embodiments, said mutations introduced in VHH_2MFR67 for humanization purposes are deletion of residue M32, substitution of E to D on position 72 and/or substitution of D to N on position 73 of SEQ ID No. 36.

In other most particular embodiments, said mutations introduced in VHH_2HFO19 for humanization purposes are deletion of residue N52 and/or of residue N53 of SEQ ID No. 8.

A humanized version of 2HFO42 is provided as the VHH with amino acid sequence as depicted in SEQ ID No. 37 or alternatively as depicted in SEQ ID No. 38-65.

Humanization can be performed using humanization techniques known in the art. In some embodiments, possible humanizing substitutions or combinations of humanizing substitutions may be determined by methods known in the art, for example without the purpose of being limiting, by a comparison between the sequence of a VHH and the sequence of a naturally occurring human VH domain. In some embodiments, the humanizing substitutions are chosen such that the resulting humanized VHHs still retain advantageous functional properties. Generally, as a result of humanization, the VHHs of the application may become more “human-like”, while still retaining favourable properties such as a reduced immunogenicity, compared to the corresponding naturally occurring VHH domains. In various embodiments, the humanized VHHs of the application can be obtained in any suitable manner known in the art and thus are not strictly limited to polypeptides that have been obtained using a polypeptide that comprises a naturally occurring VHH domain as a starting material. Indeed, modification of the amino acid sequences may be achieved using any known technique in the art e.g., site-directed mutagenesis or PCR based mutagenesis. Such techniques are described, for example, in Sambrook et al., Molecular Cloning: A Laboratory Manual, Cold Spring Harbor Press, Plainview, N.Y., 1989 and Ausubel et al., Current Protocols in Molecular Biology, John Wiley & Sons, New York, N.Y., 1989.

In various embodiments, the mutations that were introduced for example to humanize the FRα binding agent do not substantially reduce the present FRα binding agent's capability to specifically bind to the human FRα. In various embodiments, the mutations do not substantially reduce the present FRα binding agent's capability to specifically bind to FRα without neutralizing FRα.

### Association Kinetics of the FRα Binding Agents

In various embodiments, the binding affinity of the FRα binding agent of the application for the full-length and/or mature forms and/or isoforms and/or splice variants and/or fragments and/or monomeric and/or dimeric and/or tetrameric forms and/or any other naturally occurring or synthetic analogs, variants, or mutants ^{∘}including monomeric and/or dimeric and/or tetrameric forms) of human FRα may be described by the equilibrium dissociation constant ^{∘}K_{dis}), alternatively by the dissociation constant k_{off}. In various embodiments, the FRα binding agent comprises a targeting moiety that binds to the full-length and/or mature forms and/or isoforms and/or splice variants and/or fragments and/or any other naturally occurring or synthetic analogs, variants, or mutants ^{∘}including monomeric and/or dimeric and/or tetrameric forms) of human FRα with a K_{D} of less than 10 μM or more particularly of less than 1 μM and/or more than 1 nM. In other embodiments, the FRα binding agent of current application comprises a targeting moiety that binds to the full-length and/or mature forms and/or isoforms and/or splice variants and/or fragments and/or any other naturally occurring or synthetic analogues, variants, or mutants ^{∘}including monomeric and/or dimeric and/or tetrameric forms) of human FRα with a K_{dis} between 1 nM and 1 μM or between 5 nM and 950 nM or between 10 nM and 900 nM or between 20 nM and 850 nM or between 30 nM and 800 nM or between 40 nM and 700 nM or between 50 nM and 600 nM or between 50 nM and 500 nM. In a more particular embodiment said K_{dis} for human FRα is between 55 nM and 350 nM. In a most particular embodiment said K_{dis} for human FRα is between 200 nM and 350 nM, even more particularly between 250 and 300 nM. In other embodiments, the FRα binding agent comprises a targeting moiety that binds to the full-length and/or mature forms and/or isoforms and/or splice variants and/or fragments and/or any other naturally occurring or synthetic analogs, variants, or mutants ^{∘}including monomeric and/or dimeric and/or tetrameric forms) of human FRα with a K_{dis} of about 300 nM, about 250 nM, about 275 nM, about 100 nM, about 75 nM or about 50 nM.

According to another embodiment of the application, the FRα binding agent of current application has an affinity for mouse and human FRα in the range from about 1 nM to about 1 μM, or in the range from about 2 nM to about 700 nM, or in the range from about 2 nM to about 60 nM or in the range from about 20 nM to 300 nM, e.g. as measured by biolayer interferometry ^{∘}BLI) and/or ELISA.

In various embodiments, the ISVDs or VHHs of the application are not limited to a specific biological source or to a specific method of preparation. Said ISDV or VHH sequences can generally be generated or obtained by suitably immunizing a species of Camelid with a mouse and/or human FRα molecule ^{∘}i.e. so as to raise an immune response and/or heavy chain antibodies directed against FRα), by obtaining a suitable biological sample from the Camelid ^{∘}such as a blood sample, or any sample of B-cells), and by generating VHH sequences directed against FRα, starting from the sample, using any suitable known technique. VHHs can also be obtained by expressing a nucleotide sequence encoding a naturally occurring VHH domain, by “humanization” of a naturally occurring VHH domain or by expression of a nucleic acid encoding such humanized VHH domain, by using synthetic or semi-synthetic techniques for preparing proteins, polypeptides or other amino acid sequences known in the art, by preparing a nucleic acid encoding a VHH using techniques for nucleic acid synthesis known in the art, followed by expression of the nucleic acid thus obtained, and/or by any combination of one or more of the foregoing.

### Production of Folate Receptor Alpha Binding Agents

The FRα binding agents, particularly FRα antibodies, more particularly the ISVDs or VHHs of the application are not limited to a specific biological source or to a specific method of preparation. Methods for producing the FRα binding agents of the application are described herein. For example, DNA sequences encoding the FRα binding agents of the application can be easily prepared by the art-known techniques such as cloning, hybridization screening and Polymerase Chain Reaction ^{∘}PCR). Standard techniques for cloning, DNA isolation, amplification and purification, for enzymatic reactions involving DNA ligase, DNA polymerase, restriction endonucleases and the like, and various separation techniques are those known and commonly employed by those skilled in the art. A number of standard techniques are described in Sambrook et al. ^{∘}1989), Maniatis et al. ^{∘}1982), Wu ^{∘}ed.) ^{∘}1993) and Ausubel et al. ^{∘}1992). Alternatively, DNA sequences encoding the FRα binding agents of the application can be chemically synthesized using methods known in the art. Synthetic DNA sequences can be ligated to other appropriate nucleotide sequences, including for example expression control sequences, to produce gene expression constructs encoding the desired FRα binding agents.

Accordingly, in various embodiments, the present application provides for isolated nucleic acids comprising a nucleotide sequence encoding the FRα binding agents described in current application. One embodiment further discloses an expression cassette comprising said nucleic acid molecule. More specific embodiments disclose the expression cassette wherein elements for cell- or tissue-specific expression are present. Further embodiments relate to a vector comprising said expression cassette or said nucleic acid molecule. More particular, said vector may be a viral vector, even more particular a lentiviral or AAV vector.

In order to produce the FRα binding agents of current application, expression vectors comprising a nucleic acid sequence encoding said FRα binding agents can then be introduced into host cells through transfection, transformation, or transduction techniques. Hence, in various embodiments, the present application provides for a host cell comprising a nucleic acid encoding one of the FRα binding agents of the present application. For example, nucleic acids encoding the FRα binding agent of the application can be introduced into host cells by retroviral transduction. Illustrative host cells are defined herein, and include for instance E. coli cells, Chinese hamster ovary CHO) cells, yeast cells such as Pichia spp., human embryonic kidney 293 ^{∘}HEK 293) cells, HeLa cells, baby hamster kidney ^{∘}BHK) cells, monkey kidney cells ^{∘}COS), human hepatocellular carcinoma cells ^{∘}e.g., Hep G2), and myeloma cells. Transformed host cells can be grown under conditions that permit the host cells to express the genes that encode the FRα binding agent of the application.

Following expression, the FRα binding agents can be harvested and purified using techniques well known in the art, e.g. affinity tags such as glutathione-S-transferase ^{∘}GST) and histidine ^{∘}His) tags or by chromatography. Specific expression and purification conditions will vary depending upon the expression system employed. For example, if a gene is to be expressed in E. coli, it is first cloned into an expression vector by positioning the engineered gene downstream from a suitable bacterial promoter, e.g. Trp or Tac, and a prokaryotic signal sequence. In another example, if the engineered gene is to be expressed in eukaryotic host cells, e.g. CHO cells, it is first inserted into an expression vector containing for example, a suitable eukaryotic promoter, a secretion signal, enhancers, and various introns. In an embodiment, the FRα binding agent of the application comprises a His tag, a FLAG-tag and/or a Myc tag. In an embodiment, the FRα binding agent of the application comprises a His tag and a proteolytic site to allow cleavage of the His tag.

Current application thus also provides a host cell comprising one of the FRα binding agents described herein. Host cells comprising one of the nucleic acid molecules or the expression cassettes or the vectors of the application are provided herein as well. Host cells can be either prokaryotic or eukaryotic. Representative host cells that may be used with the invention include, but are not limited to, bacterial cells, yeast cells, plant cells and animal cells. Bacterial host cells suitable for use with the invention include Escherichia spp. cells, Bacillus spp. cells, Streptomyces spp. cells, Erwinia spp. cells, Klebsiella spp. cells, Serratia spp. cells, Pseudomonas spp. cells, and Salmonella spp. cells. Yeast host cells suitable for use with the invention include species within Saccharomyces, Schizosaccharomyces, Kluyveromyces, Pichia ^{∘}e.g. Pichia pastoris), Hansenula ^{∘}e.g. Hansenula polymorpha), Yarrowia, Schwaniomyces, Schizosaccharomyces, Zygosaccharomyces and the like. Saccharomyces cerevisiae, S. carlsbergensis and K. lactis are the most commonly used yeast hosts and are convenient fungal hosts. Animal host cells suitable for use with the invention include insect cells and mammalian cells ^{∘}most particularly derived from Chinese hamster ^{∘}e.g. CHO), and human cell lines, such as HeLa). Exemplary insect cell lines include, but are not limited to, Sf9 cells, baculovirus-insect cell systems ^{∘}e.g. review Jarvis 2003 Virology 310:1-7). Non-limiting examples of plant cells include tobacco cells, Arabidopsis cells, tomato cells, maize cells, algae cells, among others. The host cells may be provided in suspension or flask cultures, tissue cultures, organ cultures and the like. Alternatively, the host cells may also be transgenic animals.

Animal or mammalian host cells suitable for harboring, expressing, and producing one of the FRα binding agents of the application include Chinese hamster ovary cells ^{∘}CHO), such as CHO-K1 ^{∘}ATCC CCL-61), DG44 ^{∘}Chasin et al 1986 Som Cell Mol Genet 12:555-556; Kolkekar et al 1997 Biochemistry 36:10901-10909), CHO-K1 Tet-On cell line ^{∘}Clontech), CHO designated ECACC 85050302 ^{∘}CAMR, Salisbury, Wiltshire, UK), CHO clone 13 ^{∘}GEIMG, Genova, IT), CHO clone B ^{∘}GEIMG, Genova, IT), CHO-K1/SF designated ECACC 93061607 ^{∘}CAMR, Salisbury, Wiltshire, UK), RR-CHOK1 designated ECACC 92052129 ^{∘}CAMR, Salisbury, Wiltshire, UK), dihydrofolate reductase negative CHO cells ^{∘}CHO/−DHFR, Urlaub & Chasin 1980 PNAS 77:4216), and dp12.CHO cells ^{∘}U.S. Pat. No. 5,721,121); monkey kidney CV1 cells transformed by SV40 ^{∘}COS cells, COS-7, ATCC CRL-1651); human embryonic kidney cells ^{∘}e.g., 293 cells, or 293T cells, or 293 cells subcloned for growth in suspension culture, Graham et al 1977 J Gen Virol 36:59, or GnTI KO HEK293S cells, Reeves et al 2002 PNAS 99:13419); baby hamster kidney cells ^{∘}BHK, ATCC CCL-10); monkey kidney cells ^{∘}CV1, ATCC CCL-70); African green monkey kidney cells ^{∘}VERO-76, ATCC CRL-1587; VERO, ATCC CCL-81); mouse sertoli cells ^{∘}TM4, Mather 1980 Biol Reprod 23:243-251); human cervical carcinoma cells ^{∘}HELA, ATCC CCL-2); canine kidney cells ^{∘}MDCK, ATCC CCL-34); human lung cells W138, ATCC CCL-75); human hepatoma cells HEP-G2, HB 8065); mouse mammary tumor cells ^{∘}MMT 060562, ATCC CCL-51); buffalo rat liver cells ^{∘}BRL 3A, ATCC CRL-1442); TRI cells ^{∘}Mather, 1982, Annals NYAcad. Sci., 383:44-68); MCR 5 cells; FS4 cells. According to a particular embodiment, the cells are mammalian cells selected from Hek293 cells or COS cells.

The host cells described above can be transiently or stably transfected. Such transfection of DNA, such as nucleic acid molecules, expression cassettes or expression vectors into prokaryotic and eukaryotic cells can be accomplished via any technique known in the art, including but not limited to standard bacterial transformations, calcium phosphate co-precipitation, electroporation, or liposome mediated-, DEAE dextran mediated-, polycationic mediated-, or viral mediated transfection. For all standard techniques see, for example, Molecular Cloning: A Laboratory Manual ^{∘}Sambrook, et al., 1989, Cold Spring Harbor Laboratory Press; Culture of Animal Cells: A Manual of Basic Technique, 2* Ed. ^{∘}R. I. Freshney. 1987. Liss, Inc. New York, N.Y.). The host cell may also be a recombinant host cell, which involves a cell which has been genetically modified to contain an isolated DNA molecule, nucleic acid molecule or expression construct or vector of the invention. The DNA can be introduced by any means known to the art which are appropriate for the particular type of cell, including without limitation, transformation, lipofection, electroporation or viral mediated transduction.

Furthermore, in alternative embodiments, the use of the herein described nucleic acid molecules, expression cassettes, or vectors encoding the FRα binding agents, particularly FRα antibodies, more particularly FRα ISVDs or VHHs is provided for the production of said FRα binding agent, antibodies, ISVDs or VHHs. In a particular embodiment, said use is provided for production of an intrabody. An intracellular antibody or “intrabody” is an antibody or a fragment of an antibody that is heterologously expressed within a designated intracellular compartment, a process which is made possible through the in-frame incorporation of intracellular trafficking signals. An intrabody can be expressed in any shape or form such as an intact IgG molecule or a Fab fragment, more particularly as genetically engineered antibody fragment for example as single domain intrabodies or VHHs. For a review see Zhu, and Marasco, 2008 ^{∘}Therapeutic Antibodies. Handbook of Experimental Pharmacology 181. Ed. Springer-Verlag Berlin Heidelberg).

### Chimeras and Fusions

In various embodiments, any of the FRα binding agents of the application is provided as part of a chimera or fusion with one or more other agents. In particular embodiments, said other agent is a cytotoxic agent, a therapeutic agent, an imaging agent, radionuclide, an antisense oligonucleotide, an antibody or antibody fragment including another VHH ^{∘}also referred to herein as multivalent or multispecific agents). In other particular embodiments, said other agent is a nanoparticle, a lipid nanoparticle or an exosome. Alternatively phrased, a composition, more particularly a pharmaceutical composition is provided comprising any of the FRα binding agents of current application coupled to one or more other agents. In one embodiment, said agent is a chemical entity. The term “chemical entity” as used herein refers to simple or complex organic and inorganic molecules. Non-limiting examples of a chemical entity as used in current application is a peptide, peptidomimetic, protein, antibody ^{∘}incl. antibody fragments such as ISVDs and VHHs), carbohydrate, nucleic acid or derivative thereof, a ligand, a substrate, a phosphate, an agonist, an antagonist, a neurotransmitter, an inhibitor, a drug. In one embodiment, said chemical entity is a biological, a small molecule, a therapeutic agent, an imaging agent or a test compound.

“Biological” as used here refers to a substance that is made from a living organism or its products. A biological can be composed of sugars, proteins, or nucleic acids or complex combinations of these substances, or may be living entities such as cells and tissues. Biologics are isolated from a variety of natural sources—human, animal, or microorganism—and may be produced by biotechnology methods and other cutting-edge technologies. A non-limiting example of a biological is an antibody.

A “small molecule” as used herein ^{∘}as in the field of molecular biology and pharmacology) refers to a low molecular weight ^{∘}<900 daltons) organic compound that may regulate a biological process. Most drugs are small molecules. Larger structures such as nucleic acids and proteins, and many polysaccharides are not small molecules, although their constituent monomers ribo- or deoxyribonucleotides, amino acids, and monosaccharides, respectively) are considered small molecules. Small molecules can have a variety of biological functions or applications, serving as cell signalling molecules, drugs in medicine, pesticides in farming, and in many other roles for example by inhibiting a specific function of a protein or disrupt protein-protein interactions. These compounds can be natural ^{∘}such as secondary metabolites) or artificial ^{∘}such as peptidomimetics).

A “therapeutic agent” as used herein refers to a substance capable of slowing, interrupting, arresting, controlling, stopping, reducing or reverting the progression or severity of a sign, symptom, disorder, condition, injury, or disease, but does not necessarily involve a total elimination of all disease-related signs, symptoms, conditions, or disorders. Non-limiting examples of therapeutic agents are pharmaceutical agents, antibodies, antibody fragments, enzymes, antibiotics, antiproliferative agents, hormones, neurotransmitters, small molecules.

An “imaging agent” is a compound that has one or more properties that permit its presence and/or location to be detected directly or indirectly. Examples of such imaging agents include proteins and small molecule compounds incorporating a labelled moiety that permits detection, e.g. fluorescence or radioactivity.

The term “test compound” is used herein in the context of a “drug candidate compound” or a “candidate compound for lead optimization” in therapeutics, described in connection with the methods of the present invention. A “test compound” is thus not used as such in commercial settings but that can be used for lead optimization. These compounds comprise organic or inorganic compounds, derived synthetically or from natural resources. The compounds include polynucleotides, lipids or hormone analogues that are characterized by low molecular weights. Other biopolymeric organic test compounds include small peptides or peptide-like molecules ^{∘}peptidomimetics) comprising from about 2 to about 40 amino acids and larger polypeptides comprising from about 40 to about 500 amino acids, such as antibodies or antibody conjugates.

The above described coupling between a FRα binding agent of the application and said compound can be achieved by chemical cross-linkers or by generating fusion proteins. Covalent conjugation can either be direct or via a linker. In certain embodiments, direct conjugation is by construction of a protein fusion ^{∘}i.e. by genetic fusion of two or more genes—encoding one of the FRα binding agents of the application and one or more other proteins—and expression as a single protein). In certain embodiments, direct conjugation is by formation of a covalent bond between a reactive group on one or more portions of the FRα binding agent of the application and a corresponding group or acceptor on the chemical entity ^{∘}e.g. a neurological drug). In certain embodiments, direct conjugation is by modification ^{∘}i.e. genetic modification) of one of the two molecules to be conjugated to include a reactive group ^{∘}as non-limiting examples, a sulfhydryl group or a carboxyl group) that forms a covalent attachment to the other molecule to be conjugated under appropriate conditions. As one non-limiting example, a molecule ^{∘}i.e. an amino acid) with a desired reactive group i.e. a cysteine residue) may be introduced into, e.g. the FRα antibody and a disulfide bond formed with the chemical entity ^{∘}e.g. neurological drug). Methods for covalent conjugation of nucleic acids to proteins are also known in the art ^{∘}i.e. photocrosslinking, see e.g. Zatsepin et al 2005 Russ Chem Rev 74:77-95). Non-covalent conjugation can be by any non-covalent attachment means, including hydrophobic bonds, ionic bonds, electrostatic interactions, and the like, as will be readily understood by one of ordinary skill in the art. Conjugation may also be performed using a variety of linkers. For example, an FRα antibody and a neurological drug may be conjugated using a variety of bifunctional protein coupling agents such as Nsuccinimidyl-3-^{∘}2-pyridyldithio) propionate ^{∘}SPDP), succinimidyl-4-^{∘}N-maleimidomethyl) cyclohexane-1-carboxylate ^{∘}SMCC), iminothiolane ^{∘}IT), bifunctional derivatives of imidoesters ^{∘}such as dimethyl adipimidate HCl), active esters ^{∘}such as disuccinimidyl suberate), aldehydes ^{∘}such as glutaraldehyde), bis-azido compounds ^{∘}such as bis ^{∘}p-azidobenzoyl) hexanediamine), bis-diazonium derivatives ^{∘}such as bis-^{∘}p-diazoniumbenzoyl)-ethylenediamine), diisocyanates ^{∘}such as toluene 2,6-diisocyanate), and bis-active fluorine compounds ^{∘}such as 1,5-difluoro-2,4-dinitrobenzene). Peptide linkers, comprised of from one to twenty amino acids joined by peptide bonds, may also be used. In certain such embodiments, the amino acids are selected from the twenty naturally occurring amino acids. In certain other such embodiments, one or more of the amino acids are selected from glycine, alanine, proline, asparagine, glutamine and lysine. The linker may be a “cleavable linker” facilitating release of the chemical entity, for example upon delivery of a neurological drug to the brain or upon delivery of a therapeutic drug to a cancer cell. An acid-labile linker, peptidase-sensitive linker, photolabile linker, dimethyl linker or disulfide-containing linker Chari et al., Cancer Res. 52:127-131 ^{∘}1992); U.S. Pat. No. 5,208,020) are non-limiting examples that may be used.

According to one particular embodiment, the “coupling” can be achieved by generating a multivalent or multi-specific antibody ^{∘}e.g. a bispecific antibody). Multi-specific antibodies are ^{∘}monoclonal) antibodies or antibody fragments that have binding specificities for at least two different sites. In one embodiment, the multi-specific antibody comprises a first antigen binding site which binds the mouse and/or human FOLR1 and a second antigen binding site. In one embodiment, said second antigen binding site is a brain antigen, more particularly a brain antigen selected from the list consisting of beta-secretase 1 ^{∘}BACE1), amyloid beta, epidermal growth factor receptor ^{∘}EGFR), human epidermal growth factor receptor 2 ^{∘}HER2), Tau, apolipoprotein E4 ^{∘}ApoE4), alpha-synuclein, CD20, huntingtin, prion protein ^{∘}PrP), leucine rich repeat kinase 2 ^{∘}LRRK2), parkin, presenilin 1, presenilin 2, gamma secretase, death receptor 6 ^{∘}DR6), amyloid precursor protein ^{∘}APP), p75 neurotrophin receptor ^{∘}p75NTR) and caspase 6. In another embodiment, said second antigen binding site is a cancer antigen. A “cancer antigen” or “tumor antigen” refers to an antigenic substance produced in cancer or tumor cells, i.e. it triggers an immune response in the host. Tumor antigens are useful tumor markers in identifying tumor cells with diagnostic tests and are potential candidates for use in cancer therapy. Non-limiting examples of cancer antigens are MAGE-1, NY-ESO-1a and BAGE ^{∘}see Renkvist et al 2001 Cancer Immunology).

So, in one embodiment the FRα binding agent as described herein is a multivalent or multispecific binding agent. The binding moieties within said multivalent or multispecific agent may be of proteinaceous nature, and/or may be directly linked, or fused by a linker or spacer. The composition or binding agent^{∘}s) as described herein may appear in a “multivalent” or “multispecific” form and thus be formed by bonding, chemically or by recombinant DNA techniques, together two or more identical or different binding agents. Said multivalent forms may be formed by connecting the building blocks directly or via a linker, or through fusing the building block^{∘}s) with an Fc domain encoding sequence.

“Fc domains” or “Fc-regions” or “Fc-tails”, as interchangeably used herein, refer to the single Fc chain and/or the dimeric Fc domain of an Fc-containing proteins. Specifically in antibodies, said Fc domain is thus responsible for antibody function, and Antibody Fc engineering stands for engineering functions of antibodies, which are effector functions, such as antibody-dependent cellular cytotoxicity ^{∘}ADCC) and antibody-dependent cellular phagocytosis ^{∘}ADCP), and controlling serum half-life. Engineered Fc domains may therefore be present in the form of mutants or variants containing amino acid substitutions, insertions or deletions as to allow different modifications of the Fc in post-translational modifications, dimerization behavior, effector function, serum half life, among others. To indicate the variations present in Fc domains based on the sequence of naturally occurring IgGs, conventional antibody numbering annotations are known in the art, such as for instance IMGT numbering LeFranc, 2014; Frontiers in Immunology. 5 ^{∘}22): 1-22), Kabat numbering ^{∘}Kabat, E. A. et al., Sequences of proteins of immunological interest. 5th Edition—US Department of Health and Human Services, NIH publication no 91-3242, pp 662,680,689 ^{∘}1991)), or EU numbering ^{∘}Edelman et al. ^{∘}1969). The covalent structure of an entire gammaG immunoglobulin molecule. Proc Natl Acad Sci USA.; 63:78-85).

Non-limiting examples of multivalent constructs include “bivalent” constructs, “trivalent” constructs, “tetravalent” constructs, and so on. The immunoglobulin single variable domains comprised within a multivalent construct may be identical or different, preferably binding to the same or overlapping binding site. In another particular embodiment, the binding agent^{∘}s) of the invention are in a “multispecific” form and are formed by bonding together two or more building blocks or agents, of which at least one binds to FRα as shown herein, and at least one binds to a further target or alternative molecule, so when present in multispecific fusion, presenting a binding agent or composition that is capable of specifically binding both epitopes or targets, thus comprising binders with a different specificity. Non-limiting examples of multi-specific constructs include “bispecific” constructs, “trispecific” constructs, “tetraspecific” constructs, and so on. To illustrate this further, any multivalent or multispecific ^{∘}as defined herein) form of the invention may be suitably directed against one or more different epitopes on the same FRα antigen, or may be directed against two or more different antigens, for example one building block against FRα and one building block as a half-life extension against Serum Albumin, or another target. Multivalent or multi-specific ISVDs of the invention may also have ^{∘}or be engineered and/or selected for) increased avidity and/or improved selectivity for the desired FRα interaction, and/or for any other desired property or combination of desired properties, such as the affinity or conformational requirement, as demonstrated herein, that may be obtained by the use of such multivalent or multispecific immunoglobulin single variable domains. In another embodiment, the invention provides a polypeptide comprising any of the immunoglobulin single variable domains according to the invention, either in a monovalent, multivalent or multispecific form. Thus, polypeptides comprising monovalent, multivalent or multispecific nanobodies are included here as non-limiting examples. The multivalent or multispecific binders or building blocks may be fused directly or fused by a suitable linker, as to allow that the at least two binding sites can be reached or bound simultaneously by the multivalent or multispecific agent.

### Therapeutic and Diagnostic Application of the FRα Binding Agents of Current Application

### A Blood Brain Barrier Shuttle: FRα Binding Agents as BCSFB Transporting Agents

From all normal tissues expressing FRα, the choroid plexus epithelial cells harbour the highest level of FRα. Additionally, compared to other tissues as lung, retina, placenta, said receptor is present at the basolateral surface of CPEs that is in direct contact with folate and any FRα binding agent in the peripheral circulation. This is particularly advantageous in view of the restricted cerebral bioavailability of systemically administered pharmaceutical compounds. Indeed, transport and delivery of therapeutic agents to the brain is severely restricted by blood-brain interfaces, such as the blood-brain barrier proper or BBB and the blood-CSF barrier or BCSFB. Given that the BCSFB is formed by FRα expressing CPEs, the FRα binding agents of current application can be used to shuttle therapeutic, diagnostic or other compounds ^{∘}to which said FRα binding agents are coupled) across the BCSFB and hence improve the brain delivery of said compounds.

Hence, in one aspect of current application, any of the FRα binding agents of current application is provided for use in transporting a chemical entity across the blood brain barrier, more particularly across the BCSFB or for use in transporting a chemical entity to the brain. In line hereby, the use is provided of the FRα binding agents of current application to transport a chemical entity across the blood brain barrier, more particularly the BCSFB or to the brain. Also the use is provided of the FRα binding agents of current application to facilitate, enable, increase or improve the CNS uptake of a chemical entity across the blood brain barrier, more particularly across the BCSFB. Uptake is improved or increased when said chemical entity is statistically significantly more abundant or at least 10%, 15%, 20%, 25%, 50%, 75%, 100% or at least 2-fold, 5-fold, 10-fold, 20-fold, 50-fold or 100-fold more abundant in the brain or in the CSF in the situation that said chemical entity is coupled to one of the FRα binding agents of current application compared to the situation that said chemical entity is not coupled to one of the FRα binding agents of current application.

The FRα binding agents of current application are also provided for use as a medicament, for use in in vivo medical imaging and for use to treat a neurological disease, neuropathic pain or cancer, particularly FRα expressing cancers or to prevent brain damage after brain injury.

In current application it is demonstrated that FRα binding agents, more particularly FRα antibodies or fragments thereof, most particularly FRα binding VHHs need to fulfil certain criteria before they can be transported across the BCSFB. Concerning the VHHs of family 3 herein disclosed, it was found that a combination of epitope recognition and affinity for FRα is crucial for their BCSFB crossing ability. More particularly, FRα binding VHHs that recognize the same FRα epitope as 2HFO42 as herein disclosed need an affinity for the human FRα of less than 5×10⁻²/s, more particularly less than 4×10⁻², 3.5×10⁻², 3×10⁻², 2.9×10⁻², 2.8×10⁻², 2.7×10⁻², 2.6×10⁻², 2.5×10⁻², 2.4×10⁻², 2.3×10⁻², 2.2×10⁻², 2.15×10⁻², 2.1×10⁻², 2×10⁻², 1.9×10⁻², 1.8×10⁻², 1.7×10⁻², 1.6×10⁻², or 1.5×10⁻²/s as determined by BLI to cross the BCSFB through the FRα mediated transcytosis. In a most particular embodiment, said affinity for the human FRα is between 8×10⁻⁴/s and 4×10⁻²/s, or between 9×10⁻⁴/s and 3×10⁻²/s or between 1×10⁻³ and 2.5×10⁻²/s or between 2×10⁻³ and 2×10⁻²/s as determined by BLI. An antibody including a VHH bind its epitope through the CDR3 region. Hence, the application provides an FRα binding agent comprising a CDR3 sequence with at most two or fewer substitutions in the sequence as depicted in SEQ ID No. 5 and with an affinity for the human FRα of less than 5×10⁻²/s, more particularly less than 4×10⁻², 3×10⁻², 2×10⁻² or 1.5×10⁻²/s or even more particularly between 8×10⁻⁴ and 4×10⁻²/s or between 9×10⁻⁴/s and 3×10⁻²/s or between 1×10⁻³ and 2×10⁻²/s as determined by BLI. In a particular embodiment, said FRα binding agent does not interfere with folate binding and/or folate transport by the human FRα when binding to said FRα. In another particular embodiment, the FRα binding agent is capable of cross reacting with primate and mouse FRα. In another particular embodiment, the FRα binding agent recognizes an epitope in the human FRα comprising Q on position 141 of SEQ ID No. 1 and/or comprises a CDR2 sequence as depicted in SEQ ID No. 31 and/or a CDR1 sequence as depicted in SEQ ID No. 3. In a most particular embodiment, the FRα binding agent comprises or consists of an amino acid sequence with at least 90%, 91%, 92%, 93%, 94%, 95%, 96%, 97%, 98%, 99% or 100% identity over the full length of said sequence to SEQ ID No. 37. The FRα binding agent is also provided when coupled to a chemical entity to facilitate the uptake of the chemical entity into the cerebrospinal fluid ^{∘}CSF) across the blood CSF barrier ^{∘}BCSFB). Said chemical entity can be a biological, small molecule, therapeutic agent, a radionuclide, an antisense oligonucleotide, imaging agent or test compound. In a particular embodiment, said chemical entity is neurotensin or a neurotensin analogue. In a most particular embodiment, said FRα binding agent comprises or consists of an antibody or an antibody fragment, more particularly an immunoglobulin single variable domain or VHH.

Having unravelled the in vivo requirements for BCSFB crossing through FRα mediated transcytosis, the Applicants herein disclose a novel human blood brain barrier shuttle. Said shuttle efficiently delivers a chemical entity to the brain, more particularly to the CSF. Alternatively phrased, the application provides an FRα binding agent suitable for delivery of a chemical entity to the brain, said binding agent is one of the FRα binding agent disclosed in current application. The transport of the chemical entity to the brain is significantly increased when comparing the transport of the chemical entity without being part of the shuttle or without being to the FRα binding agent of the application. In particular embodiments, said chemical entity is a neurological disorder drug.

Said blood brain barrier shuttle comprises an FRα binding agent binding the same epitope as 2HFO42 as depicted in SEQ ID No. 2 ^{∘}more particularly comprising a CDR3 sequence with maximally two amino acids different to SEQ ID No. 5 or with maximally one amino acid different to SEQ ID No. 5 or as depicted in SEQ ID No. 5), wherein the shuttle has a dissociation constant k_{off} for human FRα of less than 5×10⁻²/s, more particularly less than 4×10⁻², 3.5×10⁻², 3×10⁻², 2.9×10⁻², 2.8×10⁻², 2.7×10⁻², 2.6×10⁻², 2.5×10⁻², 2.4×10⁻², 2.3×10⁻², 2.2×10⁻², 2.15×10⁻², 2.1×10⁻², 2×10⁻², 1.9×10⁻², 1.8×10⁻², 1.7×10⁻², 1.6×10⁻², or 1.5×10⁻²/s as determined by BLI. In one embodiment, the shuttle's k_{off} for human FRα is between 8×10⁻⁴ and 4×10⁻²/s or between 9×10⁻⁴/s and 3×10⁻²/s or between 1×10⁻³ and 2.5×10⁻²/s or between 2×10⁻³ and 2×10⁻²/s as determined by BLI. Besides the FRα binding agent, the shuttle comprises a molecule or a moiety that is to be transported to the CNS, more particularly across the BCSFB. In one embodiment said FRα binding agent of the blood-CNS-barrier shuttle comprises an ISVD with said CDR3 and wherein the CDR4 loop ^{∘}located in FR3) as defined herein comprises amino acids D at position 72 and N at position 73, according to Kabat numbering, or amino acid E at 72 and G at 73, or P at 72 and G at 73, according to Kabat numbering. In a further embodiment, said FRα binding agent of the blood-CNS-barrier shuttle comprises an ISVD further comprising a CDR2 sequence with maximally two amino acids different to SEQ ID No. 4 as for instance depicted in SEQ ID No. 32, or with maximally one amino acid different to SEQ ID No. 4, as depicted in SEQ ID No. 31, or as depicted in SEQ ID No. 4; and/or a CDR1 sequence with maximally 4 amino acids different to SEQ ID No. 3, as for instance depicted in SEQ ID No.113, or with maximally three, or two, or one amino acid different to SEQ ID No. 3 or as depicted in SEQ ID No. 3. Specifically, the ISVD of said shuttle comprises or consists of the amino acid sequence with at least 90%, at least 91%, at least 92%, at least 93%, at least 94%, at least 95%, at least 96%, at least 97%, at least 98%, at least 99% or 100% identity over the full length of said sequence to SEQ ID No. 37 ^{∘}excluding the last 9 amino acids ^{∘}3×Ala 6×His sequence). In one particular embodiment, the differences in amino acid sequence are found outside the CDR regions, so preferably in the FR regions, but outside of the CDR4 loop, and preferably different from the amino acid positions shown herein to potentially affect VHH affinity for FRα. The molecule or moiety which is part of the blood brain barrier shuttle can be a neurological disorder drug, an imaging compound, a nanoparticle or an exosome.

The blood brain barrier shuttle as described above can alternatively be phrased as a blood CNS barrier shuttle, a composition or a pharmaceutical composition, or more particularly a blood CSF barrier shuttle.

In another aspect, the blood brain barrier shuttle, blood CNS barrier shuttle, blood CSF barrier shuttle, said composition or said pharmaceutical composition is provided for use as medicament, more particular for use in the treatment or diagnosis of a neurological disorder. In one embodiment, the shuttle or composition comprises besides any of the above described FRα binding agents a neurological disorder drug, a cancer drug, a nanoparticle or an imaging compound. In a particular embodiment, the neurological disorder drug of the shuttle or composition is a biological, a small molecule, a therapeutic agent, a radionuclide, an antisense oligonucleotide or test compound.

In another particular embodiment, the composition or the shuttle is a multispecific antibody comprising said the human FRα binding agent as described above and a second antigen binding site which binds a brain antigen. Non-limiting examples of a brain antigen are beta-secretase 1 ^{∘}BACE1), amyloid beta, epidermal growth factor receptor ^{∘}EGFR), human epidermal growth factor receptor 2 ^{∘}HER2), Tau, apolipoprotein E4 ^{∘}ApoE4), alpha-synuclein, CD20, huntingtin, prion protein ^{∘}PrP), leucine rich repeat kinase 2 ^{∘}LRRK2), parkin, presenilin 1, presenilin 2, gamma secretase, death receptor 6 ^{∘}DR6), amyloid precursor protein ^{∘}APP), p75 neurotrophin receptor ^{∘}p75NTR) and caspase 6. The multispecific antibody can also comprise a second or further antigen binding site which binds a tumor or cancer antigen. Said multispecific antibody is especially advantageous in the treatment and/or diagnosis of brain cancers.

In one particular embodiment, the molecule or moiety that is part of the above-described shuttle or composition is neurotensin or a neurotensin agonist. In current application the neurotensin body temperature assay is used as an elegant system to evaluate the activity of antibodies to cross the BCSFB. However, the VHH-Neurotensin fusion described herein has clinical relevance as well. First, studies show that there is a potential therapeutic use for neurotensin ^{∘}or neurotensin agonist) induced hypothermia. Choi et al ^{∘}2012 FASEB J 26:2799-2810) showed that administration of the NT agonist ABS-201 immediately or up to 60 min after stroke attack significantly reduced infarct formation and brain cell death in an animal model of focal ischemia and was effective in promoting long-term functional recovery in post-stroke animals. Similar studies on regulated hypothermia induced by NT agonists reduce oxidative stress in the brain during reperfusion from asphyxia cardiac arrest ^{∘}Katz et al 2004 Brain Res 1017:85-91). Also, lowering body temperature with neurotensin or with a NT agonist provided a better neurologic outcome than brief external cooling in a rat model of near drowning ^{∘}Katz et al 2004 Crit Care Med 32:806-810). Therefore, the shuttle or composition as described above with a koff of between 8×10⁻⁴ and 4×10⁻²/s or between 9×10⁻⁴/s and 3×10⁻²/s or between 1×10⁻³ and 2.5×10⁻²/s or between 2×10⁻³ and 2×10⁻²/s for human FRα as determined by BLI is also provided for treating or preventing stroke, brain cell death after stroke or brain damage after brain injury. In a particular embodiment, the shuttle or composition comprises neurotensin or a neurotensin agonist.

Besides its potency in inducing hypothermia, Nemeroff et al ^{∘}1979 PNAS 76:5368-5371) demonstrated that neurotensin is an important modulator of nociceptive transmission and on a molar basis is even more potent than morphine as an antinociceptive agent. Neurotensin provides strong analgesia when administered directly into the brain and reverses pain behaviour induced by the development of neuropathic and bone cancer pain in animal models ^{∘}Demeule et al 2014 JIC 124:1199-1213). Neurotensin as part of a brain penetrable neurotherapeutic ^{∘}e.g. by coupling to one of the FRα binding agents of current application) is effective for clinical management of persistent and chronic pain. Therefore, the shuttle or composition as described herein with a koff of between 8×10⁻⁴ and 4×10⁻²/s or between 9×10⁻⁴/s and 3×10⁻²/s or between 1×10⁻³ and 2.5×10⁻²/s or between 2×10⁻³ and 2×10⁻²/s for human FRα as determined by BLI and comprising neurotensin or a neurotensin agonist is provided for use in the treatment of neuropathic pain.

A “neurological disorder” as used herein refers to a disease or disorder which affects the central nervous system or CNS and/or which has an etiology in the CNS. The “central nervous system” or “CNS” refers to the complex of nerve tissues that control physical function, and includes the brain and spinal cord. Exemplary CNS diseases or disorders include, but are not limited to neurodegenerative diseases ^{∘}including, but not limited to Lewy body disease, Parkinson's disease, tauopathies ^{∘}including, but not limited to Alzheimer's disease and supranuclear palsy)), post-poliomyelitis syndrome, Shy-Draeger syndrome, olivopontocerebellar atrophy, multiple system atrophy, striatonigral degeneration, prion diseases ^{∘}including, but not limited to bovine spongiform encephalopathy, scrapie, Creutzfeldt-Jakob syndrome, kuru, Gerstmann-Straussler-Scheinker disease, chronic wasting disease and fatal familial insomnia), bulbar palsy, dystonia ^{∘}including but not limited to DYT1 dystonia), motor neuron diseases ^{∘}including but not limited to multiple sclerosis, Charcot-Marie-Tooth ^{∘}CMT) disease, amyotrophic lateral sclerosis ALS)), and nervous system heterodegenerative disorders ^{∘}including, but not limited to Canavan disease, Huntington's disease, neuronal ceroid-lipofuscinosis, Alexander's disease, Tourette's syndrome, Menkes kinky hair syndrome, Cockayne syndrome, Halervorden-Spatz syndrome, lafora disease, Rett syndrome, hepatolenticular degeneration, Lesch-Nyhan syndrome, and Unverricht-Lundborg syndrome), dementia ^{∘}including, but not limited to Pick's disease, and spinocerebellar ataxia), cancer ^{∘}e.g. of the CNS and/or brain, including brain metastases resulting from cancer elsewhere in the body), neuropathy, amyloidosis, an ocular disease or disorder, viral or microbial infection, inflammation, ischemia, seizure, behavioral disorders, and a lysosomal storage disease.

A “neurological disorder drug” is a drug or therapeutic agent that treats one or more neurological disorder^{∘}s). Neurological disorder drugs envisage in current application include, but are not limited to antibodies, peptides, proteins, natural ligands of one or more CNS target's), modified versions of natural ligands of one or more CNS target's), aptamers, inhibitory nucleic acids or antisense oligonucleotides ^{∘}i.e., small inhibitory RNAs ^{∘}siRNA), short hairpin RNAs ^{∘}shRNA) or gapmers), ribozymes, and small molecules, or active fragments of any of the foregoing that either are themselves or specifically recognize and/or act upon ^{∘}i.e., inhibit, activate, or detect) a CNS antigen or target molecule. A “CNS antigen” or “brain antigen” is an antigen expressed in the CNS, including the brain, which can be targeted with an antibody or small molecule. Non-limiting examples of said CNS antigen or target molecule are amyloid precursor protein or portions thereof, amyloid beta, beta-secretase BACE1, gamma-secretase, Tau, alpha-synuclein, parkin, huntingtin, DR6, presenilin 1, presenilin 2, ApoE, glioma or other CNS cancer markers, and neurotrophins. Non-limiting examples of neurological disorder drugs and disorders they may be used to treat are anti-BACE1 antibodies ^{∘}e.g. WO2009121948, WO2010146058, WO2012064836) and anti-HER2 antibody ^{∘}e.g. trastuzumab) ^{∘}e.g. WO2003087131).

In another aspect of the application, a method of treating a subject is provided, said method comprising the step of administering to said patient the shuttle or ^{∘}pharmaceutical) composition described above having a koff for human FRα of less than 5×10⁻²/s, or less than 4×10⁻²/s, 3.5×10⁻², 3×10⁻², 2.9×10⁻², 2.8×10⁻², 2.7×10⁻², 2.6×10⁻², 2.5×10⁻², 2.4×10⁻², 2.3×10⁻², 2.2×10⁻², 2.15×10⁻², 2.1×10⁻², 2×10⁻², 1.9×10⁻², 1.8×10⁻², 1.7×10⁻², 1.6×10⁻², or 1.5×10⁻²/s as determined by BLI comprising a neurological disorder drug, wherein the subject is suffering from a neurological disorder. Also a method of in vivo medical imaging a body area or tissue of a subject is provided, more particularly a brain region, said method comprises administering to the subject an effective amount of any of the blood brain barrier shuttles herein disclosed comprising an imaging compound, and detecting the imaging compound in body areas of said subject. The method further comprises collecting one or more images of the subject and displaying the one or more images of the subject. The images may be taken over a period of time, including multiple images over a period of time. The collecting and displaying of said images can be done by a commercially available scanner and the accompanying computer hardware and software. For example PET and SPECT scanners may be used. Said imaging compound can be any compound that allows efficient in vivo medical imaging. A non-limiting example is a radionuclide, e.g. Technetium ^{∘}99mTC) or Lutetium-177. Also provided is a method of transporting the composition or shuttle described herein from the peripheral blood stream in a subject to the CSF, more particularly from the basolateral side of the CPE cells to the apical side, said method comprising the step of administering to said subject any one of the shuttles or ^{∘}pharmaceutical) compositions described herein.

In one embodiment of above methods, said composition or shuttle is administered to said patient using a route selected from the list consisting of oral administration, nasal administration, intravenous administration, intramuscular administration, subcutaneous administration, transdermal administration, intradermal administration, topical administration and enteral administration. In one embodiment said composition is not administered intracerebrally or intracerebroventricularly or epidurally or not through any alternative direct administration to the brain.

### Anti-Cancer Approaches: FRα-Targeting Anti-Cancer Therapeutics

Surgery, chemotherapy and radiotherapy have long been considered the best options for cancer treatment. However, these therapies are an indiscriminate warfare, coinciding with damaging side effects and failing to protect against recurring cancer cells ^{∘}Lecocq, 2019). The identification of molecular accelerators of cancer cells, such as HER2, led to the development of molecularly targeted treatments, designed to bind and override faulty molecules in cancer cells ^{∘}Lecocq, 2019). Modern cancer therapy and diagnostic is focused on targeted and thus specific delivery of high doses of chemotherapeutic drugs or diagnostic agents to tumor sites while sparing normal tissue and thus overcoming the drug's high systemic toxicity. Targeted therapies in the clinic requires high affinity, tumor-specific agents and effective targeting vehicles to deliver therapeutics to the tumor site ^{∘}Xing, 2018).

As described earlier, FRα is because of multiple reasons an attractive anti-cancer target. FRα-targeted anticancer therapeutics are primarily developed against ovarian and endometrial cancers, since these non-mucinous ^{∘}serous and endometrioid) adenocarcinomas express FRα most consistently ^{∘}Elnakat, 2004). Antitumor pro-drugs linked to FRα-affinity ligands such as folate itself or anti-FRα binding agents as those described in current application can be absorbed within FRα expressing tumor cells based on the molecular ^{∘}Trojan horses' principle. Because FRα quantitatively recycles between the cell surface and intracellular compartments, the FRα-mediated endocytosis machinery can be used as a portal of entry to deliver large payload of anti-cancer therapeutics ^{∘}Kalim et al 2017 Drug Des Devel Ther 11). Interestingly, in most proliferating healthy tissues except in the kidney, FRα expression is restricted to the luminal or apical surface of the epithelium that is out of direct contact with folate and any folate receptor-targeting agents in the circulation ^{∘}Elnakat and Ratnam 2004 Adv Drug Deliv Rev 56; Low and Kularatne 2009 Curr Opin Chem Biol). Moreover, FRα-targeting drugs do neither accumulate in the epithelial cells of the proximal tubules of the kidney thanks to a process of reabsorption in the circulation, needed to prevent loss of folate in the urine ^{∘}Sega and Low 2008 Cancer Metastasis Rev 27). Thus, systemically administered FRα-targeting drugs should not be nephrotoxic and produce minimal systemic toxicity ^{∘}Salazar and Ratnam 2007 Cancer and Metastasis Reviews 26).

The first folate-conjugated cytotoxic agent to be evaluated in tumor therapy was a maytansinoid conjugate ^{∘}Reddy et al 2007 Cancer Res 67). Since then, a series of chemotherapy agents has been conjugated to folate or anti-FRα mAb for FRα tumor targeting ^{∘}Cheung et al 2016 Oncotarget 7). Non-limiting examples are Vintafolide, folate conjugate of desacetylvin-blastinemonohydrazide ^{∘}DAVLBH), a derivative of the microtubule destabilizing agent vinblastine ^{∘}Vlahov et al 2006 Bioorg Med Chem Lett 16) and IMGN853, an anti-FRα mAb conjugated with the microtubule-stabilizing agent maytansinoid ^{∘}Ab et al 2015 Mol Cancer Ther 14), Mirvetuximab soravtansine and MOR-ab-202. Mirvetuximab soravtansine consists of maytansinoid DM4 conjugated to a humanized anti-FRα mAb via a cleavable linker ^{∘}Scaranti, 2020 #739). Preclinical studies revealed its antitumor activity and a phase I clinical trial proved it to be well-tolerated ^{∘}Scaranti, 2020). MOR-ab-202 is a new generation Ab, composed of farletuzumab conjugated with the microtubule targeting agent eribulin. MOR-ab-202, showed improved in vivo specificity and exerted enhanced durable and potent anti-tumor effects in a xenograft model ^{∘}Farran, 2019 #261).

Secondly, non-conjugated FRα-specific monoclonal antibodies ^{∘}mAbs), e.g. the fully humanized IgG1 antibody Farletuzumab, are studied for their role in passive immunotherapy. Passive anti-FRα immunotherapy is based on the administration of mAb therapy that can selectively target FRα-positive cancers ^{∘}Farran, 2019). The anti-tumor activity is attributed to antibody-dependent cellular cytotoxicity ^{∘}ADCC) ^{∘}Ebel et al 2007 Cancer Immun 7). FRα can be passively targeted with chimeric, mouse and human antibodies, alone or as conjugates for the delivery of T cells, radionuclides and cytokines to cancer tissues ^{∘}Farran, 2019). Farletuzumab has been evaluated in a phase I clinical trial and had a slow rate of clearance owing to a terminal half-life estimated to be between 121 h and 260 h ^{∘}Scaranti, 2020). Farletuzumab has also been evaluated in a phase II clinical trial in women with ovarian cancer in combination with carboplatin and taxane and a farletuzumab only maintenance therapy ^{∘}Scaranti, 2020). MOv18 is another IgG1 antibody that was generated by vaccinating mice with ovarian cancer cells ^{∘}Scaranti, 2020). The radiolabelled chimeric form was administered intravenously or intraperitoneally to ovarian cancer patients to evaluate its feasibility of radioimmunoscintigraphy and in several early phase trials this approach has been proven to be safe ^{∘}Scaranti, 2020). Next, an IgE form of MOv18 has been developed to provoke a rapid allergic hypersensitivity reaction by mast cells, and this was more effective than the IgG1 isotype in preclinical studies ^{∘}Scaranti, 2020).

In another aspect, any of the FRα binding agents of current application are provided for use in in vivo medical imaging or for use to treat cancer, particularly FRα expressing cancers, even more particularly FRα expression cancers selected from the list consisting of ovary, breast, pleura, lung, cervix, endometrium, colon, kidney, bladder and brain cancer. Current application teaches that for transport to the CNS over the BCSFB the FRα binding agents of the application should have a dissociation constant koff within a specific range. However, for binding to the FRα in or for binding at the surface of FRα expressing cancer cells, said specific dissociation constant is not an essential feature. Hence, any FRα binding agent herein disclosed is provided of use in cancer diagnostic and treatment approaches, for example by coupling to anti-cancer agents or imaging compounds.

In one embodiment, an FRα binding agent is provided with a K_{dis} from 50 nM to 500 nM for human FRα, said binding agent when coupled to a chemical entity improves the uptake of the chemical entity into FRα expressing cancer cells or improves the binding of the chemical entity to the surface of FRα expressing cancer cells. In particular embodiments, the FRα binding agent is one of the FRα binding agents from the application. In a most particular embodiment, the FRα binding agent is one of the VHHs from the application.

VHHs have been studied extensively in the context of targeted cancer therapy and immunotherapy. VHHs are embraced by different types of strategies in the fight against cancer: ^{∘}1) dampen oncogenic signals, ^{∘}2) deliver lethal punch to cancer cells, ^{∘}3) design cancer vaccines, ^{∘}4) engage cytolytic cells, and ^{∘}5) prevent immunosuppressive events ^{∘}Lecocq, 2019).

VHHs lacking antagonistic traits, yet target cancer cells, have been coupled to other technology platforms to deliver a targeted, lethal punch to cancer cells ^{∘}Lecocq, 2019). VHHs have been coupled to death inducing ligands ^{∘}e.g. TRAIL), truncated form of Pseudomonas exotoxin A, various drugs and drug-loaded nanoparticles, photosensitisers ^{∘}i.e. hitting a photosensitizer with light of a particular wavelength in an oxygenated environment results in formation of ROS), therapeutic radionuclides ^{∘}i.e. radioactive labels such as Lutetium-177, Iodine-131, Astatine-211, Actinium-225 and Bismuth-213 can be used to release their energy in the proximity of cancer cells, thereby causing irreparable DNA damage), and enzymes for prodrug activation ^{∘}e.g. β-lactamase to convert prodrug 7-^{∘}4-carboxybutanamido) cephalosporin mustard in phenylenediamine mustard) ^{∘}Lecocq, 2019). Similar as photosensitizers, branched gold nanoparticles kill cancer cells when excited by NIR-light, but by generating heat instead of ROS Lecocq, 2019). VHH can also bring these toxic moieties close to cancer cells, while minimizing toxic effects to healthy tissues, hence reducing potential adverse effects ^{∘}Lecocq, 2019).

Several bifunctional molecules have been designed ^{∘}e.g. anti-EGFR VHH coupled to TRAIL) ^{∘}Lecocq, 2019). Among the drugs that are frequently used to treat various cancer types are cisplatin and its analogues, carboplatin and oxaliplatin as well as doxorubicin, RTK inhibitors and death effector molecules. As these drugs lack selectivity, VHHs have been used to target them to cancer cells ^{∘}Lecocq, 2019).

In various embodiments, a pharmaceutical composition is provided comprising any of the FRα binding agents of current application coupled to a chemotherapeutic agent for use as a medicament, more particularly for use to treat cancer, even more particularly for use to treat FRα expressing cancers.

Non-limiting examples of said chemotherapeutic agents are alkylating agents such as thiotepa and CYTOXAN cyclosphosphamide; alkyl sulfonates such as busulfan, improsulfan and piposulfan; aziridines such as benzodopa, carboquone, meturedopa, and uredopa; ethylenimines and methylamelamines including altretamine, triethylenemelamine, trietylenephosphoramide, triethiylenethiophosphoramide and trimethylolomelamine; acetogenins ^{∘}e.g., bullatacin and bullatacinone); a camptothecin ^{∘}including the synthetic analogue topotecan); bryostatin; cally statin; CC-1065 ^{∘}including its adozelesin, carzelesin and bizelesin synthetic analogues); cryptophycins ^{∘}e.g., cryptophycin 1 and cryptophycin 8); dolastatin; duocarmycin including the synthetic analogues, KW-2189 and CB 1-TM1); eleutherobin; pancratistatin; a sarcodictyin; spongistatin; nitrogen mustards such as chlorambucil, chlornaphazine, cholophosphamide, estramustine, ifosfamide, mechlorethamine, mechlorethamine oxide hydrochloride, melphalan, novembichin, phenesterine, prednimustine, trofosfamide, uracil mustard; nitrosureas such as carmustine, chlorozotocin, fotemustine, lomustine, nimustine, and ranimnustine; antibiotics such as the enediyne antibiotics ^{∘}e.g., calicheamicin, especially calicheamicin gammaII and calicheamicin omegaII; dynemicin, including dynemicin A; bisphosphonates, such as clodronate; an esperamicin; as well as neocarzinostatin chromophore and related chromoprotein enediyne antibiotic chromophores), aclacinomysins, actinomycin, authramycin, azaserine, bleomycins, cactinomycin, carabicin, caminomycin, carzinophilin, chromomycinis, dactinomycin, daunorubicin, detorubicin, 6-diazo-5-oxo-L-norleucine, ADRIAMYCIN doxorubicin ^{∘}including morpholino-doxorubicin, cyanomorpholino-doxorubicin, 2-pyrrolino-doxorubicin and deoxy doxorubicin), epirubicin, esorubicin, idarubicin, marcellomycin, mitomycins such as mitomycin C, mycophenolic acid, nogalamycin, olivomycins, peplomycin, potfiromycin, puromycin, quelamycin, rodorubicin, streptonigrin, streptozocin, tubercidin, ubenimex, zinostatin, zorubicin; anti-metabolites such as methotrexate and 5-fluorouracil ^{∘}5-FU); folic acid analogues such as denopterin, methotrexate, pteropterin, trimetrexate; purine analogs such as fludarabine, 6-mercaptopurine, thiamiprine, thioguanine; pyrimidine analogs such as ancitabine, azacitidine, 6-azauridine, carmofur, cytarabine, dideoxyuridine, doxifluridine, enocitabine, floxuridine; androgens such as calusterone, dromostanolone propionate, epitiostanol, mepitiostane, testolactone; anti-adrenals such as minoglutethimide, mitotane, trilostane; folic acid replenisher such as frolinic acid; aceglatone; aldophosphamide glycoside; aminolevulinic acid; eniluracil; amsacrine; bestrabucil; bisantrene; edatraxate; def of amine; demecolcine; diaziquone; elformithine; elliptinium acetate; an epothilone; etoglucid; gallium nitrate; hydroxyurea; lentinan; lonidainine; maytansinoids such as maytansine and ansamitocins; mitoguazone; mitoxantrone; mopidanmol; nitraerine; pentostatin; phenamet; pirarubicin; losoxantrone; podophyllinic acid; 2-ethylhydrazide; procarbazine; PSK polysaccharide complex ^{∘}JHS Natural Products, Eugene, Oreg.); razoxane; rhizoxin; sizofuran; spirogermanium; tenuazonic acid; triaziquone; 2,2′,2″-trichlorotriethylamine; trichothecenes ^{∘}e.g., T-2 toxin, verracurin A, roridin A and anguidine); urethan; vindesine; dacarbazine; mannomustine; mitobronitol; mitolactol; pipobroman; gacytosine; arabinoside ^{∘}“Ara-C”); cyclophosphamide; thiotepa; taxoids, e.g., TAXOL paclitaxel ^{∘}Bristol-Myers Squibb Oncology, Princeton, N.J.), ABRAXANE Cremophor-free, albumin-engineered nanoparticle formulation of paclitaxel American Pharmaceutical Partners, Schaumberg, 111.), and TAXOTERE doxetaxel ^{∘}Rhone-Poulenc Rorer, Antony, France); chloranbucil; GEMZAR gemcitabine; 6-thioguanine; mercaptopurine; methotrexate; platinum analogs such as cisplatin, oxaliplatin and carboplatin; vinblastine; platinum; etoposide ^{∘}VP-16); ifosfamide; mitoxantrone; vincristine; NAVELBINE. vinorelbine; novantrone; teniposide; edatrexate; daunomycin; aminopterin; xeloda; ibandronate; irinotecan ^{∘}Camptosar, CPT-11) ^{∘}including the treatment regimen of irinotecan with 5-FU and leucovorin); topoisomerase inhibitor RFS 2000; difluoromethylornithine ^{∘}DMFO); retinoids such as retinoic acid; capecitabine; combretastatin; leucovorin LV); oxaliplatin, including the oxaliplatin treatment regimen ^{∘}FOLFOX); lapatinib ^{∘}Tykerb); inhibitors of PKC-α, Raf, H-Ras, EGFR ^{∘}e.g., erlotinib ^{∘}Tarceva)) and VEGF-A that reduce cell proliferation and pharmaceutically acceptable salts, acids or derivatives of any of the above. In addition, the methods of treatment can further include the use of photodynamic therapy.

In some embodiments, the FRα binding agents or the pharmaceutical compositions described herein act synergistically when co-administered with another therapeutic agent. In such embodiments, the FRα binding agent and the additional therapeutic agent may be administered at doses that are lower than the doses employed when the agents are used in the context of monotherapy.

### Frα-Targeting for Tumor Imaging

When diagnosing cancer, one would like to know as much as possible about the tumor, such as the presence of targetable tumor antigens and the immune context, to plan and monitor the most effective treatment ^{∘}Lecocq, 2019). FRα-targeting for non-invasive imaging of FRα positive primary and metastatic tumors allows reliable patient selection for personalized anti-cancer treatment with FRα-targeting therapeutics and permits whole-body monitoring of the FRα expression status of tumors throughout treatment ^{∘}Cheung et al 2016 Oncotarget 7). Imaging techniques based on FRα specific agents also assist surgeons in performing better resections in patients with FRα-expressing tumors ^{∘}Scaranti, 2020).

One approach is the use of FRα-targeted contrast-enhanced MRI ^{∘}Scaranti, 2020). Here, targeted folate-conjugated tracers ^{∘}e.g. dendrimer polychelate) accumulate in FRα-expressing tumors and improve contrast enhancement ^{∘}Scaranti, 2020). Other examples available in the art are ^{∘}1) folic acid coupled with a carboxylate bearing iron oxide in breast cancer, ^{∘}2) superparamagnetic iron oxide nanoparticles incorporated into heparin-folic acid micelles and ^{∘}3) radiotracers, such as radiolabelled ^{∘}e.g. 99mTc, cheaper and easier to produce than 111In) folate derivatives used whole-body SPECT analysis ^{∘}Scaranti, 2020). In clinical studies, 99mTc-ertafolitide ^{∘}peptide derivative of folic acid) is well tolerated ^{∘}Scaranti, 2020). Folic acid radioconjugates have been proposed as promising theranostic strategy for patients with FRα-positive cancers ^{∘}see further). Also non-radiolabeled approaches have been demonstrated to be successful, including fluorescent probes linked with folates enabling intraoperative visualization of tumors ^{∘}Scaranti, 2020).

Another approach is the use of labeled FRα binding agents. Antibody-based diagnostics resulted in encouraging levels of tumor-to-background resolution, for example 89Zr-DFO-M9346A ^{∘}Scaranti, 2020). Radioimmunoscintigraphy ^{∘}RIS), a strategy using radiolabeled monoclonal antibodies targeted to FRα was already used in clinical trial and showed to be successful in patients with ovarian cancer ^{∘}Crippa et al 1991 Eur J Cancer 27; van Zanten-Przybysz et al 2001 Int J Cancer 92).

VHH-based imaging has been extensively studied to detect cancer cells in preclinical studies ^{∘}e.g. antigens CEA, EGFR, HER2, PSMA, CD20, CD38) ^{∘}Lecocq, 2019). For clinical purposes, the most advanced VHH-based imaging agent is 68Ga-coupled anti-HER2 nanobody 2Rs15d for PET imaging of BC patients ^{∘}Lecocq, 2019). The first clinical trial in 2016 revealed that HER2 in primary tumors and local or distant metastases could be detected and imaged as soon as 60 min post-injection without adverse effects, such as renal toxicity and tracer-induced antibodies and was highly specific ^{∘}Lecocq, 2019). Moreover, background uptake was very low with the exception of signals observed in the kidneys, intestines and liver ^{∘}Lecocq, 2019). Recently, a phase II clinical trial evaluating the potential of 68Ga-NOTA-2Rs15d to detect brain metastasis has been initiated ^{∘}NCT03924466) ^{∘}Lecocq, 2019). Implementation of VHH-based imaging of cancer markers can be a guide for therapy selection, in particular as targeted therapies have been developed for many of these cancer markers, some of which are based on the use of VHH ^{∘}e.g. anti-HER2 VHH for targeted therapy) ^{∘}Lecocq, 2019). Moreover, VHH-based probes have been developed to image the expression of immune checkpoints ^{∘}Lecocq, 2019).

For noninvasive imaging, VHHs need to be labeled with an imaging probe that can consist of a ^{∘}1) radioisotope, ^{∘}2) fluorescent dye, ^{∘}3) microbubble or ^{∘}4) a chemical like gadolinium, allowing imaging via technologies such as single-photon emission computed tomography ^{∘}SPECT), positron emission tomography ^{∘}PET), optical imaging ^{∘}OI), ultrasound ^{∘}US) and MRI ^{∘}Lecocq, 2019). The majority of VHH-mediated imaging studies use SPECT and PET, because these radioisotope-based techniques have a high sensitivity, resolution and offer quantitative information ^{∘}Lecocq, 2019). In preclinical studies, VHHs often contain a genetically inserted C-terminal hexahistidine tag for purification purposes, which can be complexed with 99mTc ^{∘}CO3), a γ-emitting radionuclide that is easily detectable using SPECT ^{∘}Lecocq, 2019). For PET, which is clinically more relevant, VHHs are labeled with positron-emitting radionucles ^{∘}18F ^{∘}half life 68 min), 64Cu, 68Ga ^{∘}half life 110 min) and 89Zr) ^{∘}Lecocq, 2019). The half-lifes match the biological half-life of VHHs when injected i.v. ^{∘}Lecocq, 2019). Site-specific labelling is desired to obtain homogenous and consistent tracers ^{∘}e.g. transpeptidase sortase A-mediated ligation, catalizing formation of peptide bond between C-terminally expressed LPXTG peptide motif of the VHH and the N-terminal oligo-glycine motif on the label) ^{∘}Lecocq, 2019). An alternative to radiolabelling of VHHs is the use of fluorescent dyes that can be combined with optical imaging ^{∘}OI). For in vivo imaging, NIR emitting fluorophores ^{∘}e.g. IRDye-680RD or -800CW, Cy5 and AlexaFluor 680) are the label of choice, as these provide strong contrast and resolution combined with signal detection in depths ranging from several hundred um to one cm ^{∘}Lecocq, 2019). Advantages of OI are its flexibility, simplicity and cost-effective character, as in contrast to radioisotope-mediated imaging, it does not require dedicated facilities ^{∘}Lecocq, 2019). OI is often used to study surface lesions during surgical or endoscopic procedures, as OI dyes have limited tissue penetrating capacity compared to radioisotope-based imaging ^{∘}Lecocq, 2019). US can be used as an alternative to radiolabelled VHHs while retaining the ability for high-resolution images ^{∘}Lecocq, 2019). US requires conjugation of VHHs to US contrast agents, microbubbles or nanobubbles that allow the molecular characterization of the vascular wall ^{∘}after i.v. administration) ^{∘}Lecocq, 2019). In MRI imaging, VHH-coated superparamagnetic nanoparticles allow antigen detection in xenografted tumors ^{∘}Lecocq, 2019).

### FRα Binding VHHs for Use in Theranostics

A growing modality in precision oncology is the development of theranostics, as this enables patient selection, treatment and monitoring ^{∘}Lecocq, 2019). In this approach, labelled compounds and an imaging technology are used to diagnose patients and select the best treatment option, whereas for therapy, related compounds are used to target cancer cells or the tumor stroma ^{∘}Lecocq, 2019). In this context, VHHs and VHH-directed therapeutics have gained interest ^{∘}Lecocq, 2019). This interest stems from their high antigen specificity, small size, ease of labelling and engineering, allowing specific imaging and design of therapies targeting antigens on tumor cells, immune cells as well as proteins in the TME ^{∘}Lecocq, 2019).

In oncology there is a growing interest in targeted radionuclide therapy ^{∘}TRNT) that selectively delivers radioactivity and kills malignant cells, while minimizing the harm to healthy cells ^{∘}Ersahin et al., 2011). Due to the widespread availability of therapeutic radionuclides, this therapy strategy is gaining more attention ^{∘}Tomblyn et al., 2012). Radioimmunotherapy ^{∘}RIT) is a TRNT strategy that employs radiolabeled monoclonal antibodies ^{∘}mAbs) that interact with tumor-associated proteins that are expressed on the cancer cell surface and thus readily accessible by these circulating agents. For the treatment of B cell Non-Hodgkin's lymphoma ^{∘}NHL) RIT consists of the radiolabeled anti-CD20 mAbs ⁹⁰Y-ibritumomab tiuxetan ^{∘}Zevalin) and ¹³¹-tositumomab ^{∘}Bexxar). Zevalin is now FDA approved as a late-stage add-on to the unlabeled anti-CD20 mAb Rituximab for the treatment of relapse and refractory NHL. Due to the high radiosensitivity of lymphomas only a relatively low absorbed dose is required to obtain an objective response. Although recent clinical trials have shown beneficial effect of the combination of Rituximab and Zevalin versus Rituximab alone ^{∘}Tomblyn et al., 2012), Zevalin has only been approved for late-stage disease ^{∘}patients with disease recurrence or non-responders to chemotherapy and immunotherapy with Rituximab).

VHHs have superior characteristics compared to classical mAbs and their derived fragments for in vivo cell targeting ^{∘}De Vos et al., 2013). In terms of molecular imaging of cancer, VHHs have been directed to a variety of membrane-bound cancer cell biomarkers, such as CEA, EGFR, HER2, and PSMA D'Huyvetter et al., 2014). Because of their exceptional specificity of targeting, and the fact that they show to be functional after labeling with radionuclides, VHHs became valuable vehicles for nuclear imaging and TRNT ^{∘}D'Huyvetter et al., 2014).

Diagnostic tests like IHC are current practice but are unable to portray whole tumor expression levels and this is even worse for metastatic lesions ^{∘}Lecocq, 2019). Indeed, this could explain the failure to accurately predict outcome responses in all patients. Whole body, non-invasive imaging modalities such as PET, SPECT, MRI and OI, using VHH-based tracers, could fulfil these shortcomings and could be implemented repetitively without the need of collecting invasive biopsies ^{∘}Lecocq, 2019). Intriguingly, many of the described VHHs hold the potential to be used as molecular imaging probes as well as therapeutic agents. The term “theranostic” was initially put forward to describe the development of diagnostic tests alongside the application of a therapy targeted towards a specific molecular feature ^{∘}Lecocq, 2019). Currently, the term theranostics is used in a much stricter sense and rather refers to agents that are identical or closely related and that harbour the potential to be used both for diagnostic as well as for therapeutic purposes ^{∘}Lecocq, 2019). VHHs targeting cancer-specific membrane proteins ^{∘}e.g. HER2) have been evaluated for both imaging and therapeutic applications ^{∘}Lecocq, 2019). The clearest example of VHH theranostics is where both diagnostic tracers and therapeutic compounds are radiolabelled, in a TRT approach ^{∘}Lecocq, 2019). The radiolabel can be different ^{∘}Gallium-68 or Fluor-18 for PET imaging and Actinium-225 for α-TRT), but sometimes the radiolabel is the same, such as Iodine-131 labelled VHHs that are first used at low doses in SPECT imaging for diagnosis and dose estimations, and then at higher doses for TRT ^{∘}Lecocq, 2019). Of importance, diagnostic and therapeutic VHH-radiopharmaceuticals have similar pharmacokinetics and biodistribution profile ^{∘}Lecocq, 2019).

In one embodiment, any of the FRα binding agent of the application coupled to a radionuclide is provided. In an embodiment, the FRα binding agent is coupled or fused to the radionuclide either directly or through a coupling agent and/or a linker and/or a tag. In a specific embodiment, the FRα binding agent is fused to the radionuclide via a His-tag. Methods used for radiolabeling the FRα binding agent are conventional methods and are known to persons skilled in the art. Any available method and chemistry may be used for association or conjugation of the radionuclide to the FRα binding agent. As an example, tricarbonyl chemistry may be used for radiolabeling ^{∘}Xavier et al. 2012). In certain embodiments, the FRα binding agent is coupled to a radionuclide that is damaging or otherwise cytotoxic to cells and the FRα binding agent targets the radionuclide to FRα expressing cells, preferentially to cancerous cell. The radiolabelled FRα binding agent is used, for example—but not limited to—to target the damaging radionuclide to cancer tissue to preferentially damage or kill cancer cells.

According to particular embodiments, any of the FRα binding agent described herein is useful for targeted radionuclide therapy. “Targeted radionuclide therapy”, as used herein, refers to the targeted delivery of a radionuclide to a disease site and the subsequent damage of the targeted cells and adjacent cells ^{∘}bystander effect). In targeted radio-therapy, also referred to as systemic targeted radionuclide therapy ^{∘}STaRT), the biological effect is obtained by energy absorbed from the radiation emitted by the radionuclide. Non-limiting exemplary radionuclides are Iodine-131, Astatine-211, Bismuth-213, Lutetium-177 or Yttrium-86. Exemplary radionuclides that can be used to damage cells, such as cancer cells, are high energy emitters. For example, a high energy radionuclide is selected and targeted to cancer cells. The high energy radionuclide preferably acts over a short range so that the cytotoxic effects are localized to the targeted cells. In this way, radio-therapy is delivered in a more localized fashion to decrease damage to non-cancerous cells.

The present invention also pertains to the use of the FRα binding agents described herein for disease diagnosis and/or prognosis and/or treatment prediction in a subject. As non-limiting example, a subject having cancer or prone to it can be determined based on the expression levels, patterns, or profile of FRα in a test sample from the subject compared to a predetermined standard or standard level in a corresponding non-cancerous sample. In other words, FRα polypeptides can be used as markers to indicate the presence or absence of cancer or the risk of having cancer, as well as to assess the prognosis of the cancer and for prediction of the most suitable therapy.

In a further related aspect, the disclosure contemplates a pharmaceutical composition comprising any of the FRα binding agent as described herein, in association with a pharmaceutically acceptable carrier. Therefore, the FRα binding agent alone or coupled to chemical agent ^{∘}see above) may be formulated in a physiologically or pharmaceutically acceptable carrier suitable for in vivo administration. In certain embodiments, such compositions are suitable for oral, intravenous or intraperitoneal administration. In other embodiments, such compositions are suitable for local administration directly to the site of a tumor. In certain embodiments, such compositions are suitable for subcutaneous administration.

### Methods of Treatment

In another aspect of the application, a method of treating a subject is provided, said method comprising the step of administering to said patient a composition comprising one of the FRα binding agents of current application coupled to a cancer drug, wherein the subject is suffering from cancer.

Also a method of binding an FRα binding agent to a cancer tissue is provided, more particularly a FRα-expressing cancer tissue, comprising the step of administering a composition comprising one of the FRα binding agents of current application to the cancer tissue. Also a method of directing a compound to a cancer cell or tissue, more particularly a FRα-expressing cancer cell or tissue, comprising the step of administering a composition comprising the compound coupled to any of the FRα binding agents of current application to a cancer cell or tissue. In one embodiment, said cancer cell or tissue is present in a mammal, more particularly a human. In another embodiment, said cancer cell or tissue is an in vitro cancer cell or tissue. In yet another embodiment, said compound is any of the cytotoxic or chemotherapeutic compounds or any imaging compound herein described.

In one embodiment, a method of administering or transferring or directing a cancer drug or an imaging compound to a FRα expressing cancer cell is provided, comprising administering a composition to a subject comprising any of the herein disclosed FRα antibodies coupled to a cancer drug or an imaging compound.

In one embodiment of above methods, said composition is administered to said patient using a route selected from the list consisting of oral administration, nasal administration, intravenous administration, intramuscular administration, subcutaneous administration, transdermal administration, intradermal administration, topical administration and enteral administration. In one embodiment said composition is not administered intracerebrally or intracerebroventricularly or epidurally or not through any alternative direct administration to the brain.

### In Vivo Medical Imaging Method

In another aspect, the disclosure provides an in vivo medical imaging method. The method comprises administering to a subject, such as a human or non-human subject, an effective amount of the labelled FRα binding agent as described herein. The effective amount is the amount sufficient to label the desired cells and tissues so that the labelled structures are detectable over the period of time of the analysis. The method further comprises collecting one or more images of the subject and displaying the one or more images of the subject. The images may be taken over a period of time, including multiple images over a period of time. The collecting and displaying of said images is done by a commercially available scanner and the accompanying computer hardware and software. For example PET and SPECT scanners may be used. Said imaging compound can be any compound that allows efficient in vivo medical imaging. A non-limiting example is a radionuclide, e.g. Technetium ^{∘}99mTC) or Lutetium-177. Moreover, to further improve the usefulness of the images generated, CT, X-ray or MRI may be simultaneously or consecutively used to provide additional information, such as depiction of structural features of the subject. For example, dual PET/CT scanners can be used to collect the relevant data, and display images that overlay the data obtained from the two modalities. By way of example, when selecting a radionuclide for in vivo imaging, a gamma or positron emitting radionuclide or a radionuclide that decays by electron transfer may be preferred. Emissions can then be readily detected using, for example, positron emission tomography ^{∘}PET) or single photon emission computed tomography ^{∘}SPECT). Generally, it is desirable that the half-life of the radionuclide is long enough to be made and used in testing, but not so long that radioactivity lingers in the patient for a considerable period of time after the test has been performed. Moreover, the amount of radioactivity used to label can be modulated so that the minimum amount of total radiation is used to achieve the desired effect.

It is to be understood that although particular embodiments, specific configurations as well as materials and/or molecules, have been discussed herein for cells and methods according to the present invention, various changes or modifications in form and detail may be made without departing from the scope and spirit of this invention. The following examples are provided to better illustrate particular embodiments, and they should not be considered limiting the application. The application is limited only by the claims.

### EXAMPLES

### Example 1. Immunization of Alpacas with Folate Receptor Alpha (FRα) and Construction of VHH Libraries

Two llamas were subcutaneously injected on days 0, 7, 14, 21, 28 and 35, with a mixture of 50 μg human FRα ^{∘}Acro Biosystems, Cat No. FO1-H5229), 50 μg human FRα ^{∘}Sino Biological Inc., Cat No. 11241-H08H), 50 μg mouse FRα ^{∘}R&D Systems, Cat No. 6936-FR) and 50 μg mouse FRα ^{∘}Sino Biological Inc., Cat No. 50573-M08H) per injection. All proteins used for immunizations comprised a His6 tag and were emulsified with Gerbu adjuvant-P prior to injection. On day 40, blood was collected from the llamas in anti-coagulated tubes for subsequent lymphocyte preparation.

From the lymphocytes of each llama a separate VHH library was constructed to select and screen for the presence of antigen-specific VHHs. To this end, total RNA from peripheral blood lymphocytes was used as template for first strand cDNA synthesis with an oligo ^{∘}dT) primer. Using this cDNA, the VHH encoding sequences were amplified by PCR, digested with PstI and NotI, cloned into the PstI and NotI sites of the phagemid vector pMECS and transformed into E. coli to generate VHH libraries. To enrich these libraries for FRα ^{∘}or FOLR1, as used interchangeably herein)-specific VHHs, a combinatorial panning approach on recombinant human and mouse FOLR1 was used followed by random selection and screening of clones by ELISA for the presence of antigen-specific VHHs in their periplasmic extracts ^{∘}PEs). In order to gauge the cross-reactivity for human and mouse FOLR1, each clone was tested on both antigens.

### Example 2. Isolation of Human and Mouse FRα-Specific VHHs

After sequencing, 98 different binders were identified that could be grouped into 11 different families based on the sequence of their complementarity determining region ^{∘}CDR) 3. The majority of the VHHs belonged to one large family ^{∘}family 1), while the other families were made up of one to four VHHs.

Of the 98 VHHs, 91 were cross-reactive for both human and mouse FOLR1, including all members of family 1, 3, 7, 9 and 10. Family 5, 6, 8 and 11 each contained only one human FOLR1-specific VHH. Family 2 contained a single cross-reactive VHH and three human FOLR1-specific VHHs and family 4 was made up of a mouse FOLR1-specific and a human FOLR1-specific VHH. Based on these data, and making sure to include multiple VHHs with a wide range in off-rates from the large family 1, in combination with the absence of internal restriction sites in the VHH sequence, six VHHs from four different cross-reactive families were selected for further characterization ^{∘}Table 2). These VHHs were subcloned into an expression vector with a C-terminal cMyc and His8 tag, produced in E. coli BL21^{∘}DE3)pLysS cells and purified by immobilized metal affinity chromatography ^{∘}iMAC).

TABLE 2
FRα off-rates of selected, unpurified FRα binding VHHs.

| VHH | Family | FRα | PE-ELISA | k_{off} (s⁻¹) | SE k_{off} (s⁻¹) |
|---|---|---|---|---|---|
| 2HFO19 | 1 | mouse | 14.2 | 2.79E−03 | 2.21E−05 |
| | | human | 13.8 | 1.30E−02 | 5.77E−05 |
| 2MFR7 | | mouse | 14.1 | 5.42E−02 | 8.99E−04 |
| | | human | 12.4 | 1.08E−01 | 8.11E−04 |
| 3HFO26 | | mouse | 4.9 | 1.35E−02 | 1.51E−04 |
| | | human | 4.8 | 5.82E−02 | 4.24E−04 |
| 2HFO42 | 3 | mouse | 12.2 | 8.81E−03 | 4.56E−05 |
| | | human | 11.7 | 2.66E−02 | 2.63E−04 |
| 3HFO28 | 7 | mouse | 34.9 | 4.25E−03 | 3.05E−05 |
| | | human | 35.6 | 8.27E−03 | 1.33E−04 |
| 3MFR60 | 10 | mouse | 4.9 | 2.87E−03 | 3.38E−05 |
| | | human | 4.8 | 4.49E−02 | 6.52E−04 |

After panning of core76/77 on human and mouse folate receptor alpha ^{∘}FRα), 95 colonies were randomly selected from the second and third rounds and analyzed by ELISA on both human and mouse FRα for the presence of FRα-specific VHHs in their periplasmic extracts ^{∘}PE). This led to the identification of 98 hits. For these 98 hits, the off-rate ^{∘}k_{off}) in the PE was determined by biolayer interferometry ^{∘}BLI) on both mouse and human FRα that was directly immobilized onto AR2G biosensors. The off-rates were calculated using a 1:1 binding model. For the PE-ELISA, the ratio between the signal from the human or mouse FRα coated well and the uncoated, blocked well ^{∘}negative control) is displayed. Abbreviations: SE: standard error.

### Example 3. FRα Binding VHHs Bind Recombinant and Native FRα and do not Compete for Folic Acid (FA) Binding

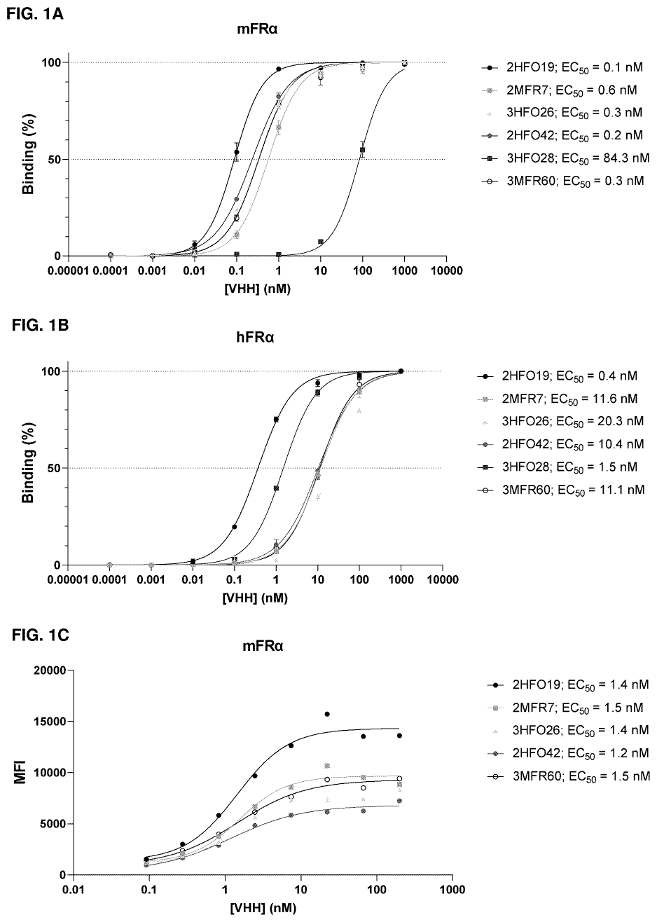

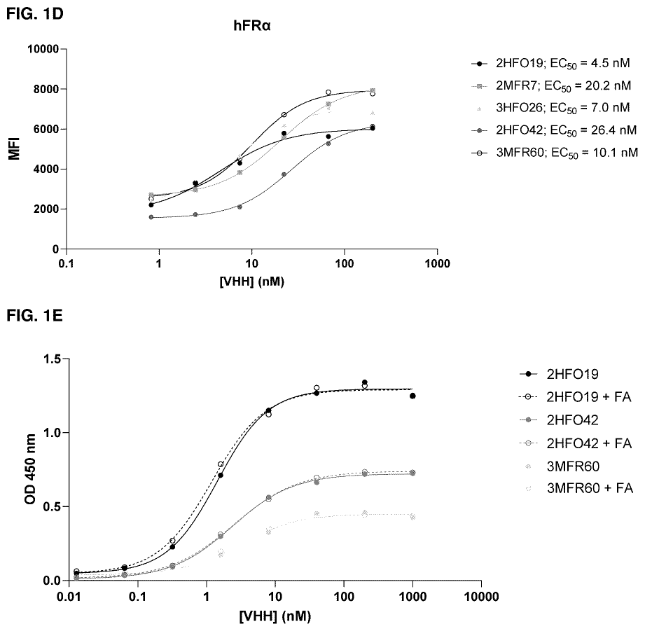

In a first step, binding affinity and cross-reactivity of the purified VHHs to human and mouse FRα was assessed by ELISA, using detection of the cMyc tag ^{∘}FIG. 1A-B). All VHHs bound to human and mouse FRα with an EC₅₀ in the nM to pM range comparable to the natural affinities of folic acid^{∘} oxidized folate; <1 nM) and 5-methyltetrahydrofolate ^{∘}reduced folate; 1-10 nM) ^{∘}Wang et al 1992 Biochemical pharmacology 44:1898-1901; Gates et al 1996 Clinical cancer research 2:1135-1141; Kamen and Caston 1986 Biochemical pharmacology 35:2323-2329) for human FRα. Most of the tested VHHs showed stronger binding to mouse FRα compared to human FRα. 2HFO19 ^{∘}family 1) was the best binder of all tested VHHs to both mouse and human FRα with an EC₅₀ of 0.1 nM and 0.4 nM respectively ^{∘}FIG. 1A-B). 2HFO42 ^{∘}family 3) and 3MFR60 ^{∘}family 10) had binding affinities comparable to 2MFR7 ^{∘}family 1) for both human and mouse FRα. Next, binding to human and mouse folate receptor beta ^{∘}FRβ encoded by FOLR2, and as used interchangeably herein) was investigated to assess the specificity of the VHHs, since there is a high level of sequence conservation between FRα and FRβ. In contrast to FOLR1, FOLR2 is mainly expressed in myeloid cells such as neutrophils and macrophages ^{∘}O'Shannessy et al 2015 Journal of ovarian research 8:29; Ross et al 1999 Cancer 85:348-57). None of the VHHs bound to human FRβ ^{∘}data not shown), indicating that the VHHs are highly specific for FRα, reducing the possibility of off-target effects.

As a next step, the kinetic binding constants were determined using BLI ^{∘}Table 3). Within the panel, 2HFO19 and 3MFR60 had the highest affinity for mouse FRα with an equilibrium dissociation constant ^{∘}K_{dis} or K_{D}) of 2.97 nM and 2.59 nM, respectively. The lowest K_{D} was observed for 2HFO42 ^{∘}23.8 nM) which is still a good binder. Similar to the ELISA results ^{∘}FIG. 1A-B), higher binding affinities were measured for mouse FRα than human FRα, with K_{D} values increasing one order of magnitude in most cases. 3HFO26 and 3MFR60 even showed and almost 100-fold gap in K_{D} between mouse and human FRα, going from 7.86 nM to 662 nM and from 2.59 nM to 169 nM respectively. For 3HFO28, binding to human FRα could not be confirmed by BLI, and this VHH was excluded from further characterization.

TABLE 3
Binding kinetics of FRα binding VHHs on recombinant FRα.

| | mFRα | | | hFRα | | |
|---|---|---|---|---|---|---|
| VHH | kₒₙ ^{°}M⁻¹s⁻¹) | k_{off} ^{°}s⁻¹) | K_{D} ^{°}M) | kₒₙ ^{°}M⁻¹s⁻¹) | k_{off} ^{°}s⁻¹) | K_{D} ^{°}M) |
| 2HFO19 | 1.32E+06 | 3.93E−03 | 2.97E−09 | 2.33E+05 | 1.49E−02 | 6.37E−08 |
| 2MFR7 | 3.15E+06 | 5.27E−02 | 1.67E−08 | 1.20E+06 | 8.49E−02 | 7.09E−08 |
| 3HFO26 | 2.50E+06 | 1.97E−02 | 7.86E−09 | 2.75E+05 | 1.82E−01 | 6.62E−07 |
| 2HFO42 | 2.03E+05 | 4.67E−03 | 2.38E−08 | 1.01E+05 | 2.76E−02 | 2.74E−07 |
| 3HFO28 | 4.64E+05 | 5.82E−03 | 1.26E−08 | NA | 1.36E−03 | NA |
| 3MFR60 | 1.15E+06 | 2.97E−03 | 2.59E−09 | 9.38E+05 | 1.59E−01 | 1.69E−07 |

The on- and off-rate ^{∘}kₒₙ and k_{off} respectively) and equilibrium dissociation constant ^{∘}K_{D}) of the anti-folate receptor alpha ^{∘}FRα) VHHs as determined by biolayer interferometry ^{∘}BLI) on both mouse and human ^{∘}m/h) FRα that was directly immobilized onto AR2G biosensors at 10 μg/ml. The kinetic parameters were calculated using a 1:1 binding model.

Furthermore, since binding to solid phase immobilized recombinant material does not necessarily reflect the native conformation of the antigen, binding to FRα expressed on the cell membrane was assessed by flow cytometry. HEK293T cells that do not endogenously express FRα were transiently transfected with mouse or human FOLR1, and dose-dependent VHH binding was detected based on the His tag ^{∘}FIG. 1C-D). All VHHs were able to bind cell-expressed mouse FRα with EC₅₀ values in the low nM range and native human FRα with EC₅₀ values ranging from 4.5 nM to 26.4 nM.

As interference with the endogenous transport of folates via FRα could have serious implications on brain homeostasis, competition between representative family members and FA for mouse FRα binding was assessed via ELISA ^{∘}FIG. 1E). After pre-incubation of the receptor with an excess of FA to ensure complete receptor saturation, the tested VHHs retained full binding capacity, showing that they do not compete with FA for mouse FRα binding.

### Example 4. FRα Binding VHHs Bind Partially Overlapping Epitopes

VHHs that share high sequence identity in the CDR3 region, a VHH family, are derived from the same B-cell lineage and diverged from each other due to somatic hypermutation or affinity maturation. Typically, VHHs from the same family recognize the same epitope on the target, but may differ in other characteristics ^{∘}De Genst et al 2006 PNAS 103:4586-4591; De Gents et al 2005 J Biol Chem 280:14114-14121). To study whether the VHHs from the different families bound different epitopes, epitope binning was performed using a tandem binding approach in BLI. One VHH from each family was allowed to bind to mouse FRα on the biosensor, followed by binding of the second VHH ^{∘}Table 4).

TABLE 4
Epitope binning of FRα binding VHHs.

| | | VHH in 1st association | | | |
|---|---|---|---|---|---|
| | | 2HFO19 | 2HFO42 | 3MFR60 | 2MF832 |
| VHH in 2nd | 2HFO19 | 0% | 12% | −7% | 100% |
| association | 2HFO42 | 1% | 0% | −2% | 41% |
| | 3MFR60 | 6% | 34% | 0% | 75% |
| | 2MFR32 | 100% | 100% | 100% | 0% |

Competition between VHHs from different complementarity determining region ^{∘}CDR) 3 families was assessed in a tandem binding assay using biolayer interferometry ^{∘}BLI). Streptavidin biosensors were loaded with biotinylated mouse FRα ^{∘}Acro Biosystems, Cat.nr. FO1-M82E9), followed by binding of anti-FRα VHHs ^{∘}VHH in first association). The biosensors were then transferred to a solution containing a second VHH mixed 1:1 with the first VHH ^{∘}VHH in second association). Using the FortéBio Data Analysis Software the absolute additional binding signal for the second association on top of the first association was calculated. The data were normalized per VHH in first association using the following formula: ^{∘}signal-self)/^{∘}maximum-self) with self being the value of the VHH in first association in combination with the same VHH in second association. The values are displayed as percentages.

2MFR32 ^{∘}SEQ ID NO: 96) showed additional binding with all of the other VHHs, indicating that it recognizes a separate epitope on mFRα. 2HFO19, 2HFO42, and 3MFR60 did not simultaneously bind to mFRα and showed competition in all the different combinations, suggesting that they bind to similar epitopes. These epitopes can either be identical, partially overlapping or in mutually exclusive conformations, since for FRβ different conformational states were reported ^{∘}Wibowo et al 2013 PNAS 110:15180-15188). The VHHs can thus be pooled into two distinct epitope bins: 2HFO19, 2HFO42, and 3MFR60 that recognize ^{∘}partially) overlapping epitopes and 2MFR32 that binds a different epitope.

### Example 5. Human FRα Q141 is Essential for Binding of the Anti-FRα VHHs

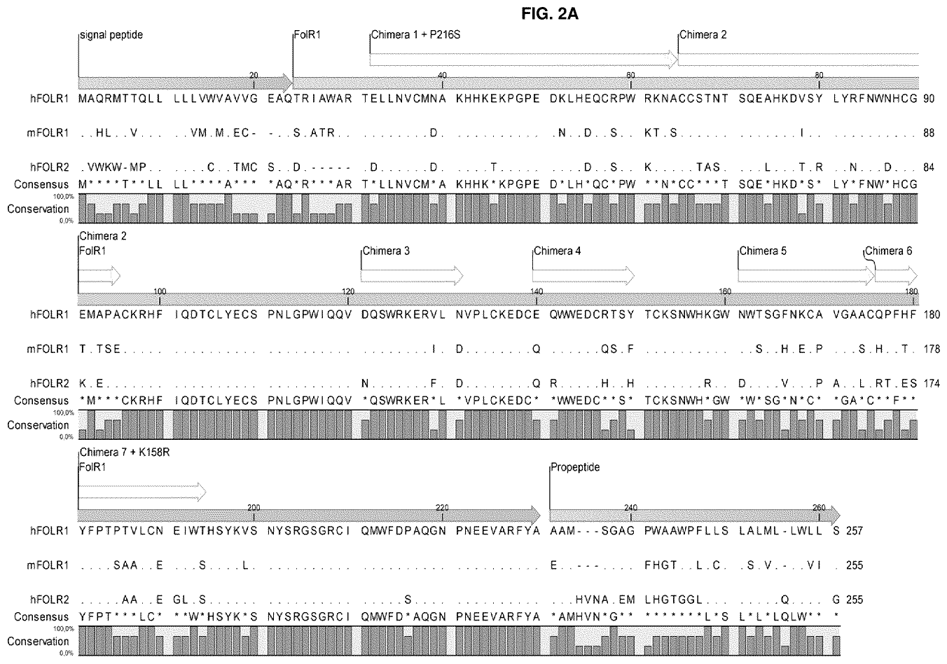

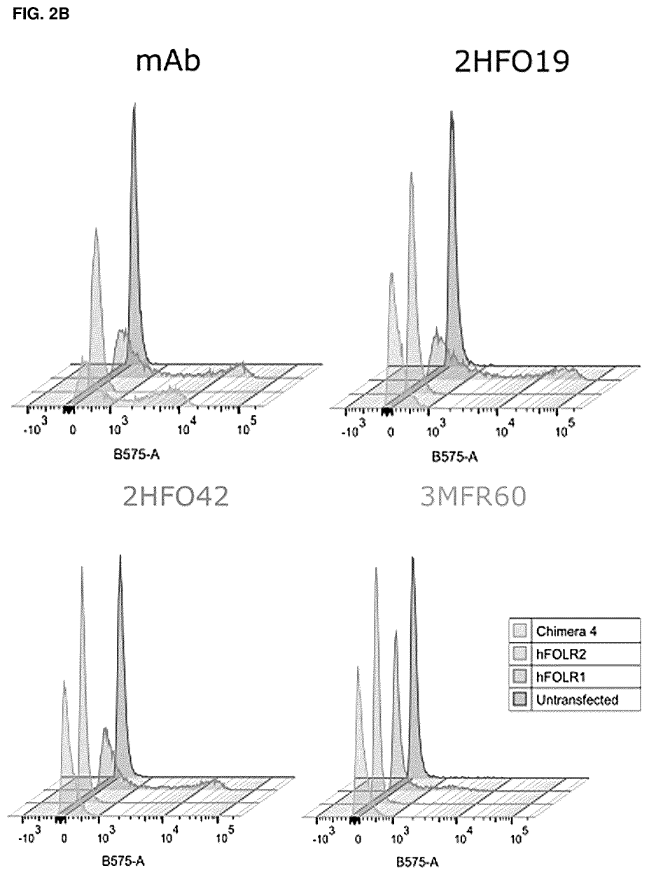

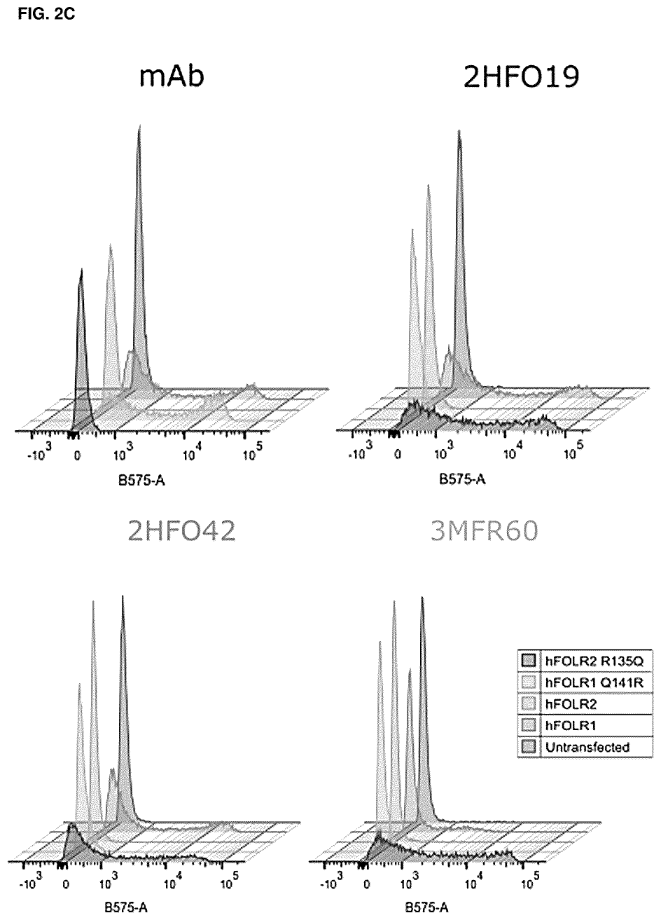

To identify the exact region in which the VHHs bind to FRα, epitope mapping experiments were performed. Since the VHHs are able to bind human FRα, but not FRβ, we generated seven human FOLR1/2 chimeric constructs where regions of human FOLR1 were replaced by the corresponding human FOLR2 ^{∘}FRβ) regions ^{∘}FIG. 2A): chimera 1 SEQ ID No. 67), chimera 2 ^{∘}SEQ ID No. 68), chimera 3 ^{∘}SEQ ID No. 69), chimera 4 ^{∘}SEQ ID No. 70), chimera 5 ^{∘}SEQ ID No. 71), chimera 6 ^{∘}SEQ ID No. 72) and chimera 7 ^{∘}SEQ ID No. 73). Three additional chimera 1 constructs were made: chimera 1a ^{∘}replacing fragment 31-40; SEQ ID No. 74), chimera 1b ^{∘}replacing fragment 41-50; SEQ ID No. 75) and chimera 1c ^{∘}replacing fragment 51-64; SEQ ID NO. 76). The constructs were transiently transfected into HEK293T cells and dose-dependent binding of the VHHs to the wild-types FRα and FRβ ^{∘}WTs) or the chimeric constructs was assessed via flow cytometry via detection of the tag ^{∘}FIG. 2B). Expression of the constructs was verified with an anti-FRα antibody ^{∘}mAb) and most of the chimeras showed similar expression levels. Chimera 6 ^{∘}SEQ ID No. 72) could not be detected with the mAb, but the tested VHHs did show binding, indicating that this region is the epitope of the antibody ^{∘}data not shown). Different anti-FRα VHHs were able to bind to cell-expressed FRα-FRβ chimeras to a similar level as WT FRα, with the exception of chimera 4 ^{∘}SEQ ID No. 70) where the binding was completely abolished for all VHHs. This implies that this region ^{∘}amino acids 140-150) plays an important role in the binding of the VHHs to their target.

To further narrow down which specific residues might play a role, single residues of human FRα in the chimera 4 region that are not conserved between FRα and FRβ were mutated to their human FRβ counterpart. While all VHHs retained binding to human FRα E140Q SEQ ID No. 77), R147H ^{∘}SEQ ID No. 79), and Y150H ^{∘}SEQ ID No. 80) ^{∘}data not shown), binding was completely abolished when the Q141R mutation ^{∘}SEQ ID No. 78) was introduced ^{∘}FIG. 2C). To verify the role of this residue in the binding of the VHHs, the R135 residue in human FRβ was replaced by glutamine ^{∘}SEQ ID No. 81) and binding of the VHHs was analyzed. Surprisingly, introduction of this single residue in human FRβ was sufficient for the VHHs to regain binding capacity. In addition to having ^{∘}partially) overlapping epitopes, the VHHs thus all require residue Q141 for binding.

### Example 6. Anti-FRα VHH 2HFO42 is Able to Cross the Blood-CSF Barrier In Vivo

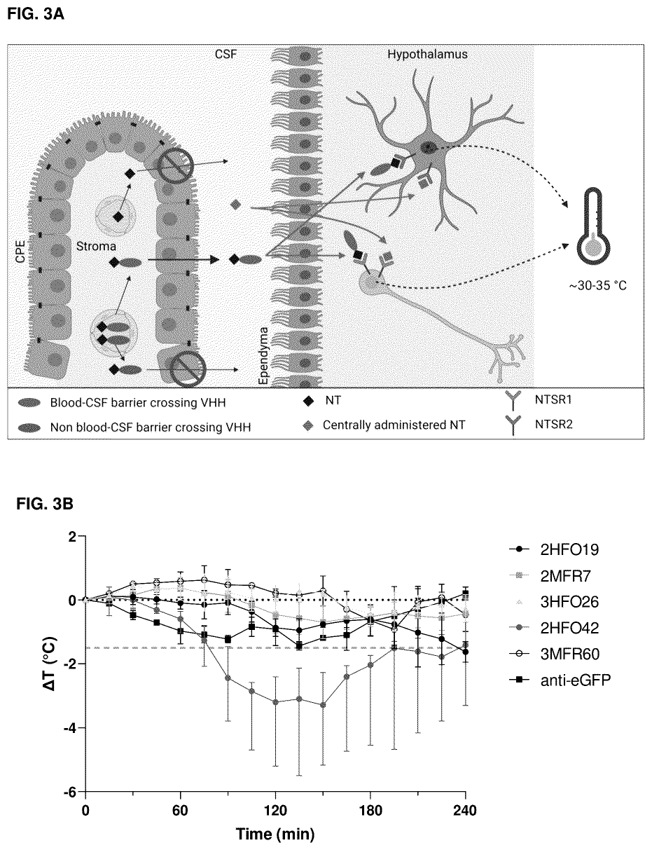

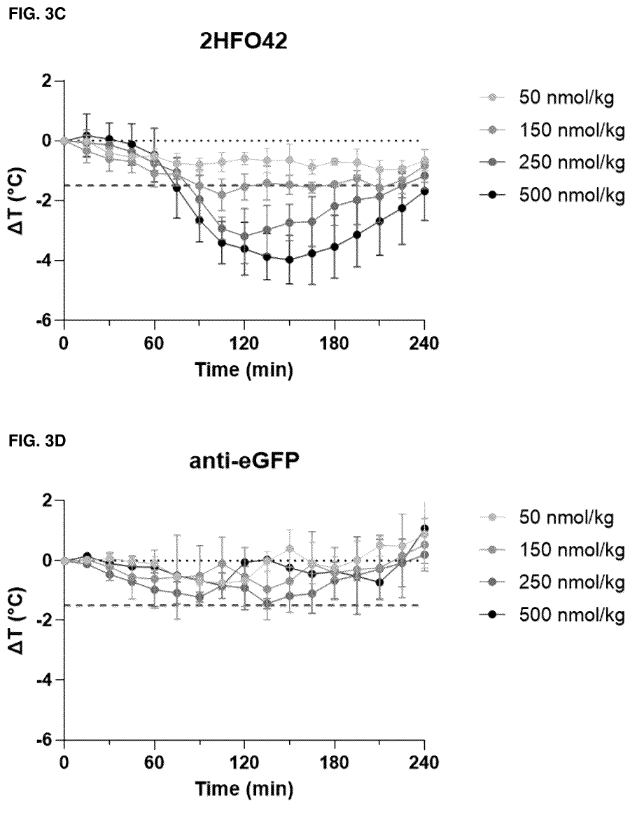

To investigate the in vivo blood-CSF barrier crossing potential of the human-mouse FRα cross-reactive VHHs, we set up an in vivo screening as described previously in Wouters et al ^{∘}2022 Fluids and barriers of the CNS; 19^{∘}1):79). In short, the FRα binding VHHs were genetically fused to the neuroactive tridecapeptide neurotensin ^{∘}NT; FIG. 3A). Upon engagement of the neurotensin receptor ^{∘}NTSR) in the hypothalamus after central administration, NT causes a measurable hypothermic effect in mice ^{∘}Bissette et al 1976 Nature 262:607-609; Nemeroff et al 1979 PNAS 76:5368-5371; Prange et al 1979 Pharmacology, biochemistry, and behavior 11:473-477). This hypothermic effect is mediated by NT binding to both neuronal NTSR1 and astrocytic NTSR2 ^{∘}Tabarean 2020 Neuropharmacology 171:108069). However, upon peripheral administration, NT as such is not able to cross the brain barriers ^{∘}Pardridge 1998 Journal of neurochemistry 70:1781-1792). Therefore, the presence of a hypothermic effect after intravenous ^{∘}iv) injection of a VHH-NT fusion protein, indicates barrier crossing potential of the VHH followed by target engagement of the NT. Since FOLR1 is only expressed at the CPE cells forming the blood-CSF interface and not at the BBB ^{∘}Weitman et al 1992 Cancer research 52:3396-3401; Weitman et al 1992 Cancer research 52:6708-6711; O'Shannessy et al 2011 Oncotarget 2:1227-1243; Grapp et al 2013 Nature Comm 4:2123), a drop in body temperature suggests that the VHHs reach the brain by crossing the blood-CSF barrier rather than the BBB.

To analyse the in vivo crossing capacity, the different human/mouse cross-reactive anti-FRα VHH-NT and control VHH-NT ^{∘}anti-eGFP VHH) fusion proteins were injected iv at a dose of 250 nmol/kg ^{∘˜}4.2 mg/kg) in TLR4−/− mice, and the body temperature was measured continuously using implanted temperature probes until 4 hours after the injection ^{∘}FIG. 3B). Of the tested VHHs, only 2HFO42-NT showed a clear drop in body temperature compared to the negative control VHH, indicating receptor engagement by NT in the brain and implying blood-CSF barrier crossing potential of 2HFO42. The integrity of the VHH-NT fusion proteins was verified by mass spectrometry analysis ^{∘}data not shown), confirming that the absence of a drop in body temperature was due to lack of crossing potential and not due to loss of NT. Next, we studied the dose dependency of the hypothermic response. 2HFO42-NT and anti-eGFP-NT were injected iv at doses of 50, 150, 250, and 500 nmol/kg and body temperature was measured ^{∘}FIG. 3C-D). For 2HFO42, the magnitude of the drop in body temperature increased with increasing doses, while the anti-eGFP control VHH was unable to induce a body temperature drop at all tested doses, confirming that there is no passive uptake in the CNS.

These results show that from the tested human-mouse cross-reactive anti-FRα VHHs, only 2HFO42 is able to cross the blood-CSF barrier. This indicates that the ability of crossing the BCSFB is not solely related to the affinity of the VHH towards the mFRα, since 2HFO19 ^{∘}family 1) and 2HFO42 ^{∘}family 3) have similar k_{off} values ^{∘}3.93×10⁻³/s versus 4.67×10⁻³/s respectively) and 2MFR7 ^{∘}family 1) and 2HFO42 ^{∘}family 3) have similar K_{D} values 1.67×10⁻⁸ versus 2.38×10⁻⁸ respectively). Interestingly, other members of the 2HFO42 family 3 neither induced hypothermia in mice, indicating that also the epitope alone is neither the sole determining factor for the BCSFB crossing ability.

### Example 7. Binding Off-Rate Requirements for Blood-CSF Barrier Crossing of Family 3 VHHs

Within VHH family 3, 2HFO42 is the only member identified so far to cross the BCSFB in mice, meaning that crossing ability relies also on other characteristics besides the CDR3 sequence and epitope recognition. It was therefore hypothesized that the binding kinetic parameters would impact the receptor mediated transcytosis and crossing ability, similar to what is known for BBB-crossing compounds.

The binding results obtained by ELISA, BLI and FACS allowed to rank the VHHs according to their optimum affinity for the mFRα, with the affinity of 2HFO9 ^{∘}fam 3) to mFRα being too low ^{∘}K_{D} 213 nM; koff 8.2×10⁻³/s), while that of 2MFR67 too high ^{∘}K_{D} 4.3 nM; koff 9.87×10⁻⁴/s) to allow functional crossing, as compared to 2HFO42 ^{∘}K_{D} 23.3 nM; koff 2.22×10⁻³/s). Since the sequences of the 3 family members are conserved in CDR3, the residues in 2HFO9 and 2MFR67 that differ with respect to 2HFO42 were systematically mutated to the corresponding residue of 2HFO42 in all possible combinations to investigate whether an affinity more similar to that of 2HFO42 determines the crossing capacity in the NT model.

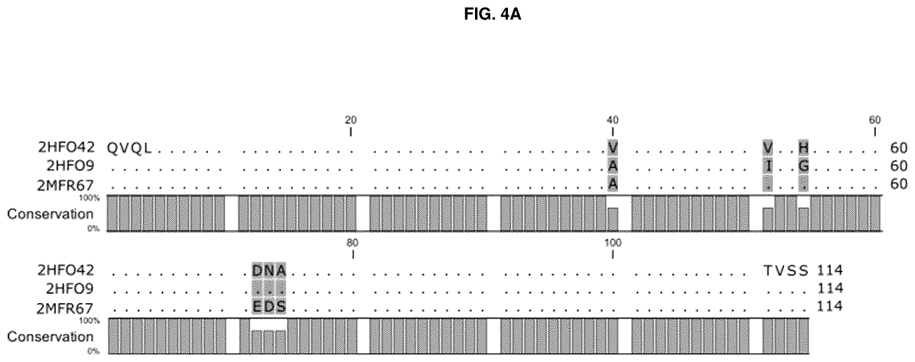

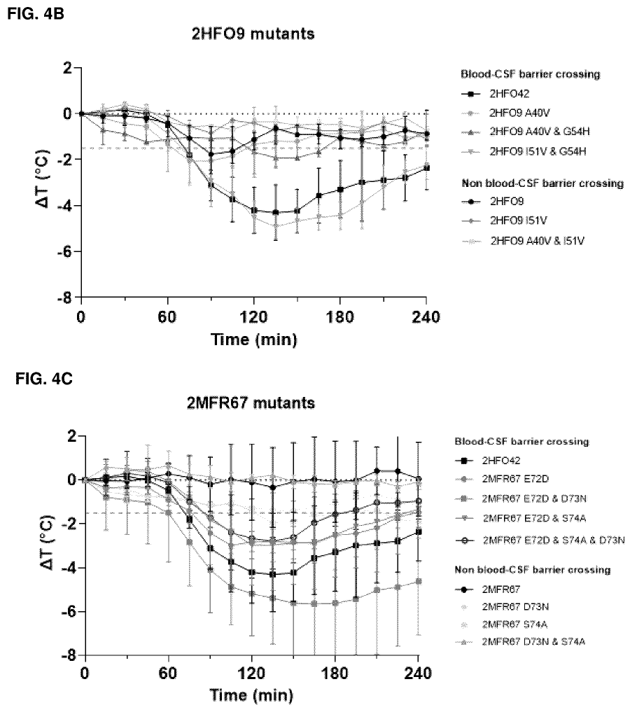

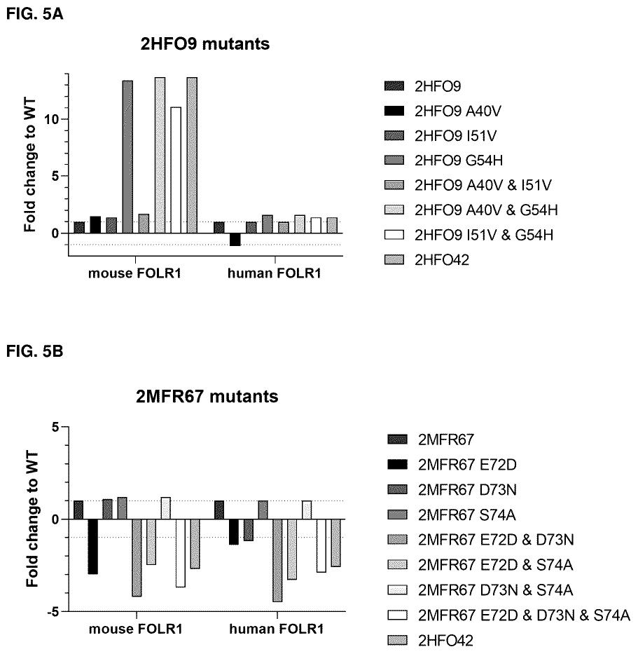

2HFO9 differs from 2HFO42 in three residues at positions 40, 51 and 54 ^{∘}according to Kabat numbering) ^{∘}FIG. 4A). The different single, double and triple mutants were generated and produced as a NT fusion protein in E. coli as described in Wouters et al. Fluids Barriers CNS. 2022; 19^{∘}1):79). Integrity of the fusion proteins was verified by SDS-PAGE and mass spectrometry analysis, and binding to human and mouse FRα was determined by ELISA ^{∘}data not shown) and BLI ^{∘}FIG. 5A). Looking at the off-rates for the 2HFO9 variants on mouse FRα, introduction of G54H in both single and double mutant form lead to improvement in off-rate ^{∘}k_{off} 2.04 10⁻³ to 2.36×10⁻³/s), bringing it in the same range as the 2HFO42 off-rate. In contrast, introduction of A40V and/or I51V did not affect the off-rate.

Next, the different mutant NT-fusions were checked for their BCSFB crossing ability in TLR4−/− mice by IV injection at a dose of 250 nmol/kg and the change in body temperature was measured. In contrast to 2HFO9, the 2HFO9^{∘}I51V-G54H) variants with k_{off} values similar to 2HFO42 showed a drop in body temperature similar to 2HFO42-NT ^{∘}FIG. 4B). 2HFO9^{∘}G54H)-NT production was not successful in this experiment, but its crossing capacity was later confirmed using a HEK293-F cell-based production. 2HFO9^{∘}I51V) and 2HFO9^{∘}A40V, I51V) did not show a clear drop in body temperature, meaning that these VHHs are not able to cross to the brain. These results indicate that the presence of H54 is favourable for crossing and that it increases the affinity to FRα.

2MFR67 differs with 2HFO42 at four positions 40, and 72 to 74. It was found that the E72D mutation decreased the affinity of 2MFR67 on mouse FRα for the single, double, and triple mutant forms to around a 2.5- to 4-fold ^{∘}FIG. 5B), resulting in off-rates in the same range as that of 2HFO42. Introduction of the E72D mutation in 2MFR67 leads to BCSFB crossing of the single, double and triple mutant form as determined by a clear drop in body temperature upon iv injection ^{∘}FIG. 4C). In contrast, mutants D73N and S74A in 2MFR67 were not able to cross the BCSFB in mice as determined by the lack of body temperature drop when fused to NT. These results indicate that the presence of A74 is unfavourable for crossing, and that A74 slightly increases the affinity to FRα.

In conclusion, results with point mutants suggest that within the VHHs of family 3 the off-rate is an important determinant, and point to a narrow window in K_{D}/k_{off}-rates for optimal dissociation rates that support BCSFB crossing of family 3 VHH variants in the mouse system. An overview of the k_{off} value for mouse and human FRα can be found in Table 5 as well as the BCSFB crossing ability of the NT-fusions in mice.

TABLE 5
Binding of family 3 VHH variants to the mouse and human FOLR1 by BLI.

| | Mouse Fol1R | | | Human Fol1R | | | BCSFB |
|---|---|---|---|---|---|---|---|
| | | Fold to | Fold to | | Fold to | Fold | crossing |
| ID | koff ^{°}s − 1) | WT | 2HF042 | koff ^{°}s − 1) | WT | hu/ms | in mice |
| 2HFO42-NT | 2.22E−03 | | 1.0 | 1.54E−02 | | 7 | +++ |
| 2HFO9-NT | 8.2 E−03 | | 0.3 | 2.35E−02 | | 3 | − |
| 2MFR67-NT | 9.87E−04 | | 2.3 | 5.98E−03 | | 6 | − |
| 2HFO9 | 2.36E−03 | 0.3 | 0.9 | 1.45E−02 | 0.7 | 6 | +++ |
| ^{°}I51V,G54H)-NT | | | | | | | |
| 2HFO9^{°}G54H)-NT | 2.15E−03 | 0.3 | 1.0 | 1.33E−02 | 0.6 | 6 | + |
| 2HFO9^{°}I51V)-NT | 1.20E−02 | 1.8 | 0.2 | 2.05E−02 | 1.0 | 2 | − |
| 2HFO9 ^{°}A40A, I51V) | 1.13E−02 | 1.6 | 0.2 | 1.28E−02 | 0.6 | 1 | − |
| 2HFO9 | 2.04E−03 | 0.3 | 1.1 | 2.11E−02 | 1.0 | 10 | + |
| ^{°}A40V, G54H)-NT | | | | | | | |
| 2HFO9^{°}A40V) -NT | 1.24E−02 | 1.8 | 0.2 | 2.36E−02 | 1.1 | 2 | + |
| 2MFR67 ^{°}E72D)-NT | 2.49E−03 | 2.5 | 0.9 | 2.43E−02 | 4.1 | 10 | ++ |
| 2MFR67-NT ^{°}D73N) | 1.06E−03 | 1.1 | 2.1 | 7.41E−03 | 1.2 | 7 | − |
| 2MFR67-NT ^{°}S74A) | 9.26E−04 | 0.9 | 2.4 | 5.99E−03 | 1.0 | 6 | − |
| 2MFR67 | 2.90E−03 | 2.9 | 0.8 | 2.71E−02 | 4.5 | 9 | +++ |
| ^{°}E72D, D73N)-NT | | | | | | | |
| 2MFR67 | 1.01E−03 | 1.0 | 2.2 | 6.23E−03 | 1.0 | 6 | − |
| ^{°}D73N, S74A)-NT | | | | | | | |
| 2MFR67 | 2.34E−03 | 2.4 | 1.0 | 2.00E−02 | 3.3 | 9 | ++ |
| ^{°}E72D, S74A)-NT | | | | | | | |
| 2MFR67 | 2.58E−03 | 2.6 | 0.9 | 1.73E−02 | 2.9 | 7 | ++ |
| ^{°}E72D, D72N, S74A)- | | | | | | | |
| NT | | | | | | | |

### Example 8. 2HFO42 and 2MFR67 is Enriched in Non-Human Primate CSF Upon Peripheral Administration

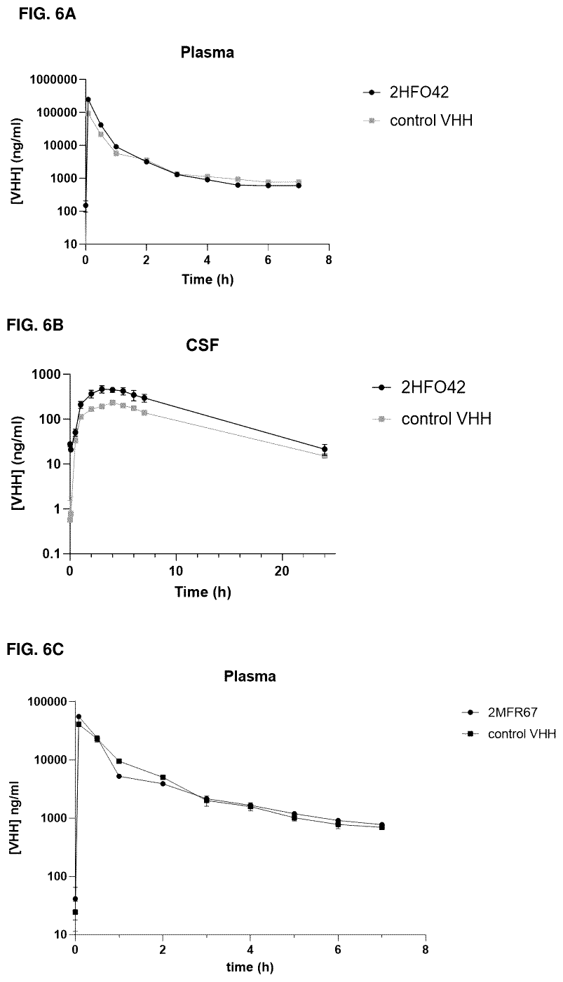

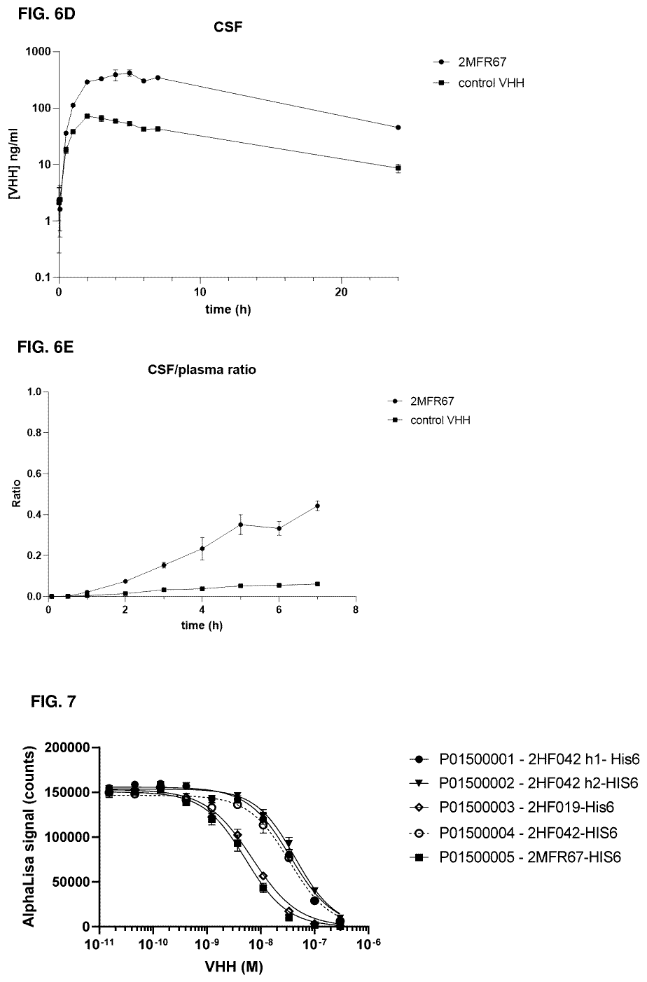

In a first step towards translation to the clinic, we assessed whether 2HFO42 that crosses the BCSFB in mice has also blood-CSF barrier crossing potential in non-human primates. First, binding to rhesus macaque FRα was confirmed by ELISA ^{∘}Table 6). Next, a rhesus macaque was implanted with a catheter close to the cisterna magna for CSF isolation. To analyse the CSF uptake over time, a mixture of 2HFO42 and an irrelevant VHH was iv injected at a dose of 8 mg/kg for each VHH and serial sampling of plasma and CSF ^{∘}FIG. 6A-B). The bioanalysis of VHHs was done via ELISA on the respective antigens, and detection of the tags. Both VHHs showed similar pharmacokinetic profiles in plasma, with a half-life of around 20 min, in line with expectations for a non-half-life extended VHH. Strikingly, higher 2HFO42 VHH levels were measured in the CSF compared to the control VHH levels, with detectable in the CSF 24 hours after the injection. These data demonstrate that 2HFO42 is able to retain blood-CSF barrier crossing capacity across different species.

2MFR67, a family member of 2HFO42, is not able to cross the blood-cerebrospinal fluid ^{∘}CSF) barrier in mice and its affinity for mFRα is much higher as compared to 2HFO42. However, its affinity and dissociation rate for human and rhesus monkey FRα ^{∘}Table 6) are in the range of 2HFO42 on mouse FRα ^{∘}Table 5). To assess whether this dissociation rate allows blood-CSF barrier crossing of 2MFR67 in non-human primates, the VHH together with a control VHH was tested according to the same procedure as explained above for 2HFO42 ^{∘}Error!Reference source not found.). Both 2MFR67 and control VHH showed a comparable kinetic profile in plasma with similar amounts of VHH detected upon intravenous administration ^{∘}FIG. 6C). Plasma levels of both 2MFR67 and the control VHH were below the lower limit of quantification at 24 h upon injection, indicating that the VHHs are cleared from circulation at that time point. In the CSF, clearly higher levels of 2MFR67 were detected compared to the control VHH, although the control VHH could also be detected in the CSF ^{∘}FIG. 6D). The CSF to plasma ratio of the VHH amount was also higher for 2MFR67 compared to the control VHH, indicating that the higher levels of 2MFR67 in the CSF were not due to a higher level in the plasma ^{∘}FIG. 6E). These data show that, although 2MFR67 does not show blood-CSF barrier crossing in mice, it is able to reach the CSF in rhesus monkey upon peripheral administration, indicating that this VHH has the optimal dissociation rate for blood-CSF barrier crossing in non-human primates and by extension humans and that the optimal dissociation rates for BCSFB crossing in non-human primates and mice overlap.

TABLE 6
Binding kinetics of 2HFO42 and 2MFR67 on recombinant FOLR1.

| | K_{D} ^{°}M) | SE K_{D} | kₒₙ ^{°}1/Ms) | SE kₒₙ | k_{off} ^{°}1/s) | SE k_{off} |
|---|---|---|---|---|---|---|
| 2HFO42 h2 | | | | | | |
| ^{°}SEQ ID No.42) | | | | | | |
| rhFolR1 | 2.77E−08 | 1.11E−09 | 5.77E+05 | 2.22E+04 | 1.60E−02 | 1.74E−04 |
| mFOLR1 | 6.77E−09 | 1.07E−10 | 3.67E+05 | 5.62E+03 | 2.49E−03 | 9.71E−06 |
| 2MFR67 | | | | | | |
| ^{°}SEQ ID No.44) | | | | | | |
| rhFolR1 | 1.38E−08 | 1.47E−10 | 3.15E+05 | 3.11E+03 | 4.34E−03 | 1.76E−05 |
| hFOLR1 | 8.78E−09 | 1.43E−10 | 3.53E+05 | 5.48E+03 | 3.10E−03 | 1.49E−05 |
| mFOLR1 | 2.52E−09 | 2.54E−11 | 2.27E+05 | 1.06E+03 | 5.71E−04 | 5.11E−06 |

The on- and off-rate ^{∘}kₒₙ and k_{off} respectively) and equilibrium dissociation constant ^{∘}K_{D}) of the anti-folate receptor alpha ^{∘}FOLR1) VHHs 2HFO42 and 2MFR67 was determined by biolayer interferometry ^{∘}BLI) on rhesus monkey, mouse and human ^{∘}rh/m/h) after capture of the VHH using an anti-FLAG tag antibody on mouse Fc biosensors. The kinetic parameters were calculated using a 1:1 binding model.

### Example 9. Sequence Optimisation of 2HFO42 Towards Human FRα

Given that the optimal affinity range for BCSFB crossing differs between mice and human, it was decided to set up an in vitro transcytosis assay using human CPE cells ^{∘}see Example 11). First, we set off to construct a plethora of sequences based on the current VHH data, and tested the impact of said variants on the affinities for human FRα. These optimized sequences are in particular humanized and sites prone to post-translational modifications were removed. During the design, it was intended to further optimize for human FRα binding, while retaining specificity, so avoid binding to human FRβ. Sequences can be found in Table 7.

TABLE 7
Sequences of optimized FRa binding VHHs.

| SEQ | | Description of VHH | |
|---|---|---|---|
| ID No. | ID construct | and substituted aa | Sequence |
| 2 | | 2HFO42 | QVQLQESGGGLVQAGGSLRLSCAASGSGFSGMIMGWYRQVPGKQRELV |
| | | | ATVTSHGTTNYADSVKGRFTISRDNAKNTVLLQMNSLKPEDTAVYYCNAH |
| | | | FPGIYWGKGTQVTVSS |
| 6 | | 2HF09 | QVQLQESGGGLVQAGGSLRLSCAASGSGFSGMIMGWYRQAPGKQRELV |
| | | | ATITSGGTTNYADSVKGRFTISRDNAKNTVLLQMNSLKPEDTAVYYCNAHF |
| | | | PGIYWGKGTQVTVSS |
| 36 | | 2MFR67 | QVQLQESGGGLVQAGGSLRLSCAASGSGFSGMIMGWYRQAPGKQRELV |
| | | | ATVTSHGTTNYADSVKGRFTISREDSKNTVLLQMNSLKPEDTAVYYCNAHF |
| | | | PGIYWGKGTQVTVSS |
| 37 | P01500001 = | 2HF042°Q1E, Q5V, | EVQLVESGGGLVQPGGSLRLSCAASGSGFSGMIMGWYRQAPGKQRELV |
| | humanised | A14P, V40A, L79Y, | ATVTSHGTTNYADSVKGRFTISRDNAKNTVYLQMNSLRPEDTAVYYCNAH |
| | variant 1 | K83R, Q108L)-HIS6 | FPGIYWGKGTLVTVSSAAAHHHHHH |
| | °h1) | | |
| | 2HFO42- | | |
| | His6 | | |
| 38 | P01500002 = | 2HF042°Q1E, Q5V, | EVQLVESGGGLVQPGGSLRLSCAASGSGFSGMIMGWYRQAPGKQ |
| | h2 variant | A14P, V40A, L79Y, | RELVATVTSHGTTNYADSVKGRFTISRDNAKNTVYLQMNSLRPEDT |
| | 2HFO42 | K83R, K105Q, | AVYYCNAHFPGIYWGQGTLVTVSSAAAHHHHHH |
| | | Q108L)-HIS6 | |
| 39 | P01500003 | 2HF019°Q1E, Q5V, | EVQLVESGGGLVQPGGSLRVSCAASGFPFSTVYMSWVRQAPGKG |
| | | Q108L)-HIS6 | LEWVSGINNDGGVIDYADSVKGRFTISRDNAKNTLYLQLNSLKPED |
| | | | TALYFCARGRSFVATLSSSLRGQGTLVTVSSAAAHHHHHH |
| 40 | P01500004 | 2HF042°Q1E, Q5V, | EVQLVESGGGLVQAGGSLRLSCAASGSGFSGMIMGWYRQVPGK |
| | | Q108L)-HIS6 | QRELVATVTSHGTTNYADSVKGRFTISRDNAKNTVLLQMNSLKPED |
| | | | TAVYYCNAHFPGIYWGKGTLVTVSSAAAHHHHHH |
| 41 | P01500005 | 2MFR67°Q1E, Q5V, | EVQLVESGGGLVQAGGSLRLSCAASGSGFSGMIMGWYRQAPGK |
| | | Q108L)-HIS6 | QRELVATVTSHGTTNYADSVKGRFTISREDSKNTVLLQMNSLKPED |
| | | | TAVYYCNAHFPGIYWGKGTLVTVSSAAAHHHHHH |
| 42 | P01500006 | 2HF042°Q1E, Q5V, | EVQLVESGGGLVQPGGSLRLSCAASGSGFSGMIMGWYRQAPGKQ |
| | | A14P, V40A, L79Y, | RELVATVTSHGTTNYADSVKGRFTISRDNAKNTVYLQMNSLRPEDT |
| | | K83R, Q108L)- | AVYYCNAHFPGIYWGKGTLVTVSSAAADYKDHDGDYKDHDIDYKD |
| | | FLAG3-HIS6 | DDDKGAAHHHHHH |
| 43 | P01500007 | 2HF042°Q1E, Q5V, | EVQLVESGGGLVQPGGSLRLSCAASGSGFSGMIMGWYRQAPGKQ |
| | | A14P, V40A, L79Y, | RELVATVTSHGTTNYADSVKGRFTISRDNAKNTVYLQMNSLRPEDT |
| | | K83R,K105Q, | AVYYCNAHFPGIYWGQGTLVTVSSAAADYKDHDGDYKDHDIDYK |
| | | Q108L)-FLAG3- | DDDDKGAAHHHHHH |
| | | HIS6 | |
| 44 | P01500008 | 2MFR67°Q1E, Q5V, | EVQLVESGGGLVQAGGSLRLSCAASGSGFSGMIMGWYRQAPGK |
| | | Q108L)-FLAG3- | QRELVATVTSHGTTNYADSVKGRFTISREDSKNTVLLQMNSLKPED |
| | | HIS6 | TAVYYCNAHFPGIYWGKGTLVTVSSAAADYKDHDGDYKDHDIDYK |
| | | | DDDDKGAAHHHHHH |
| 45 | P01500019 | 2HF09°Q1E, Q5V, | EVQLVESGGGLVQPGGSLRLSCAASESPFSGMIMGWYRQAPGKQ |
| | | G26E, G28P, 151V, | RELVATVTSHGTTNYADSVKGRFTISREGAKNTVYLQMNSLRPEDT |
| | | G54H, D72E, N73G, | AVYYCNAHFPGIYWGKGTLVTVSSAAAHHHHHH |
| | | Y79L,K83R,Q108L | |
| | | )-HIS6 | |
| 46 | P01500024 | 2HF09°Q1E, Q5V, | EVQLVESGGGLVQPGGSLRLSCAASGSGFSGMIMGWYRQAPGKQ |
| | | I51V, G54H, | RELVATVTSHGTTNYADSVKGRFTISRDNAKNTVYLQMNSLRPEDT |
| | | L79Y, K83R, Q108L)- | AVYYCNAHFPGIYWGKGTLVTVSSAAADYKDHDGDYKDHDIDYKD |
| | | FLAG3-HIS6-GS- | DDDKGAAHHHHHHGGGGSRRPYIL |
| | | NT°°8-13) | |
| 47 | P01500041 | P01500001°D72P, | EVQLVESGGGLVQPGGSLRLSCAASGSGFSGMIMGWYRQAPGKQ |
| | | N73G)-FLAG3- | RELVATVTSHGTTNYADSVKGRFTISRPGAKNTVYLQMNSLRPEDT |
| | | HIS6 | AVYYCNAHFPGIYWGKGTLVTVSSAAADYKDHDGDYKDHDIDYKD |
| | | | DDDKGAAHHHHHH |
| 48 | P01500042 | P01500001°G28T, | EVQLVESGGGLVQPGGSLRLSCAASGSTFIGMIMGWYRQAPGKQ |
| | | S301)-FLAG3-HIS6 | RELVATVTSHGTTNYADSVKGRFTISRDNAKNTVYLQMNSLRPEDT |
| | | | AVYYCNAHFPGIYWGKGTLVTVSSAAADYKDHDGDYKDHDIDYKD |
| | | | DDDKGAAHHHHHH |
| 49 | P01500043 | P01500001°G28T, | EVQLVESGGGLVQPGGSLRLSCAASGSTFSGMLMGWYRQAPGKQ |
| | | 133L, D72E)- | RELVATVTSHGTTNYADSVKGRFTISRENAKNTVYLQMNSLRPEDT |
| | | FLAG3-HIS6 | AVYYCNAHFPGIYWGKGTLVTVSSAAADYKDHDGDYKDHDIDYKD |
| | | | DDDKGAAHHHHHH |
| 50 | P01500044 | P01500001°S301, | EVQLVESGGGLVQPGGSLRLSCAASGSGFIGMIMGWYRQAPGKQ |
| | | D72E, N73G)- | RELVATVTSHGTTNYADSVKGRFTISREGAKNTVYLQMNSLRPEDT |
| | | FLAG3-HIS6 | AVYYCNAHFPGIYWGKGTLVTVSSAAADYKDHDGDYKDHDIDYKD |
| | | | DDDKGAAHHHHHH |
| 51 | P01500045 | P01500001°G26E, | EVQLVESGGGLVQPGGSLRLSCAASESGFSGMLMGWYRQAPGKQ |
| | | 133L)-FLAG3-HIS6 | RELVATVTSHGTTNYADSVKGRFTISRDNAKNTVYLQMNSLRPEDT |
| | | | AVYYCNAHFPGIYWGKGTLVTVSSAAADYKDHDGDYKDHDIDYKD |
| | | | DDDKGAAHHHHHH |
| 52 | P01500046 | P01500001°N73P) | EVQLVESGGGLVQPGGSLRLSCAASGSGFSGMIMGWYRQAPGKQ |
| | | -FLAG3-HIS6 | RELVATVTSHGTTNYADSVKGRFTISRDPAKNTVYLQMNSLRPEDT |
| | | | AVYYCNAHFPGIYWGKGTLVTVSSAAADYKDHDGDYKDHDIDYKD |
| | | | DDDKGAAHHHHHH |
| 53 | P01500047 | P01500001°G26E, | EVQLVESGGGLVQPGGSLRLSCAASESTFIGMLMGWYRQAPGKQ |
| | | G28T, S301, I33L)- | RELVATVTSHGTTNYADSVKGRFTISRDNAKNTVYLQMNSLRPEDT |
| | | FLAG3-HIS6 | AVYYCNAHFPGIYWGKGTLVTVSSAAADYKDHDGDYKDHDIDYKD |
| | | | DDDKGAAHHHHHH |
| 54 | P01500048 | P01500001°S30I, | EVQLVESGGGLVQPGGSLRLSCAASGSGFIGMIMGWYRQAPGKQ |
| | | D72P)-FLAG3- | RELVATVTSHGTTNYADSVKGRFTISRPNAKNTVYLQMNSLRPEDT |
| | | HIS6 | AVYYCNAHFPGIYWGKGTLVTVSSAAADYKDHDGDYKDHDIDYKD |
| | | | DDDKGAAHHHHHH |
| 55 | P01500049 | P01500001°G26E) | EVQLVESGGGLVQPGGSLRLSCAASESGFSGMIMGWYRQAPGKQ |
| | | -FLAG3-HIS6 | RELVATVTSHGTTNYADSVKGRFTISRDNAKNTVYLQMNSLRPEDT |
| | | | AVYYCNAHFPGIYWGKGTLVTVSSAAADYKDHDGDYKDHDIDYKD |
| | | | DDDKGAAHHHHHH |
| 56 | P01500050 | P01500001°G28T, | EVQLVESGGGLVQPGGSLRLSCAASGSTFSGMIMGWYRQAPGKQ |
| | | D72P)-FLAG3- | RELVATVTSHGTTNYADSVKGRFTISRPNAKNTVYLQMNSLRPEDT |
| | | HIS6 | AVYYCNAHFPGIYWGKGTLVTVSSAAADYKDHDGDYKDHDIDYKD |
| | | | DDDKGAAHHHHHH |
| 57 | P01500051 | P01500001°D72E, | EVQLVESGGGLVQPGGSLRLSCAASGSGFSGMIMGWYRQAPGKQ |
| | | N73G)-FLAG3- | RELVATVTSHGTTNYADSVKGRFTISREGAKNTVYLQMNSLRPEDT |
| | | HIS6 | AVYYCNAHFPGIYWGKGTLVTVSSAAADYKDHDGDYKDHDIDYKD |
| | | | DDDKGAAHHHHHH |
| 58 | P01500052 | P01500001°V51R, | EVQLVESGGGLVQPGGSLRLSCAASGSGFSGMIMGWYRQAPGKQ |
| | | D72E)-FLAG3- | RELVATRTSHGTTNYADSVKGRFTISRENAKNTVYLQMNSLRPEDT |
| | | HIS6 | AVYYCNAHFPGIYWGKGTLVTVSSAAADYKDHDGDYKDHDIDYKD |
| | | | DDDKGAAHHHHHH |
| 59 | P01500053 | P01500001°S301, | EVQLVESGGGLVQPGGSLRLSCAASGSGFIGMLMGWYRQAPGKQ |
| | | 133L, N73P)- | RELVATVTSHGTTNYADSVKGRFTISRDPAKNTVYLQMNSLRPEDT |
| | | FLAG3-HIS6 | AVYYCNAHFPGIYWGKGTLVTVSSAAADYKDHDGDYKDHDIDYKD |
| | | | DDDKGAAHHHHHH |
| 60 | P01500054 | P01500001°I33L, | EVQLVESGGGLVQPGGSLRLSCAASGSGFSGMLMGWYRQAPGK |
| | | N73P)-FLAG3- | QRELVATVTSHGTTNYADSVKGRFTISRDPAKNTVYLQMNSLRPED |
| | | HIS6 | TAVYYCNAHFPGIYWGKGTLVTVSSAAADYKDHDGDYKDHDIDYK |
| | | | DDDDKGAAHHHHHH |
| 61 | P01500055 | P01500001°V51R, | EVQLVESGGGLVQPGGSLRLSCAASGSGFSGMIMGWYRQAPGKQ |
| | | D72E, N73G)- | RELVATRTSHGTTNYADSVKGRFTISREGAKNTVYLQMNSLRPEDT |
| | | FLAG3-HIS6 | |
| | | | AVYYCNAHFPGIYWGKGTLVTVSSAAADYKDHDGDYKDHDIDYKD |
| | | | DDDKGAAHHHHHH |
| 62 | P01500056 | P01500001°133L, | EVQLVESGGGLVQPGGSLRLSCAASGSGFSGMLMGWYRQAPGK |
| | | D72P, N73G)- | QRELVATVTSHGTTNYADSVKGRFTISRPGAKNTVYLQMNSLRPED |
| | | FLAG3-HIS6 | TAVYYCNAHFPGIYWGKGTLVTVSSAAADYKDHDGDYKDHDIDYK |
| | | | DDDDKGAAHHHHHH |
| 63 | P01500057 | P01500001°G26E, | EVQLVESGGGLVQPGGSLRLSCAASESPFSGMIMGWYRQAPGKQ |
| | | G28P, D72E, | RELVATVTSHGTTNYADSVKGRFTISREGAKNTVYLQMNSLRPEDT |
| | | N73G)-FLAG3- | AVYYCNAHFPGIYWGKGTLVTVSSAAADYKDHDGDYKDHDIDYKD |
| | | HIS6 | DDDKGAAHHHHHH |
| 64 | P01500058 | P01500001°G26E, | EVQLVESGGGLVQPGGSLRLSCAASESPFSGMIMGWYRQAPGKQ |
| | | G28P)-FLAG3- | RELVATVTSHGTTNYADSVKGRFTISRDNAKNTVYLQMNSLRPEDT |
| | | HIS6 | AVYYCNAHFPGIYWGKGTLVTVSSAAADYKDHDGDYKDHDIDYKD |
| | | | DDDKGAAHHHHHH |
| 65 | P01500059 | P01500001°G26E, | EVQLVESGGGLVQPGGSLRLSCAASESGFSGMIMGWYRQAPGKQ |
| | | D72E)-FLAG3- | RELVATVTSHGTTNYADSVKGRFTISRENAKNTVYLQMNSLRPEDT |
| | | HIS6 | AVYYCNAHFPGIYWGKGTLVTVSSAAADYKDHDGDYKDHDIDYKD |
| | | | DDDKGAAHHHHHH |
| 82 | P01500031 | P01500001°G26E, | EVQLVESGGGLVQPGGSLRLSCAASGSGFSGILMGWYRQAPGKQ |
| | | G28P, M321, | RELVATVTSHGTTNYADSVKGRFTISRPGAKNTVYLQMNSLRPEDT |
| | | D72E, N73G - | AVYYCNAHFPGIYWGKGTLVTVSSAAADYKDHDGDYKDHDIDYKD |
| | | Flag3-His6-NT8- | DDDKGAAHHHHHHGGGGSRRPYIL |
| | | 13 | |
| 83 | P01500032 | P01500001°G26E, | EVQLVESGGGLVQPGGSLRLSCAASGSGFSGPLMGWYRQAPGKQ |
| | | G28P, M32P, D72E, | RELVATVTSHGTTNYADSVKGRFTISRPGAKNTVYLQMNSLRPEDT |
| | | N73G-Flag3- | AVYYCNAHFPGIYWGKGTLVTVSSAAADYKDHDGDYKDHDIDYKD |
| | | His6-NT8-13 | DDDKGAAHHHHHHGGGGSRRPYIL |
| 84 | P01500019 | 2HF09°Q1E, Q5V, | EVQLVESGGGLVQPGGSLRLSCAASESPFSGMIMGWYRQAPGKQ |
| | | A14P, G26E, G28P, | RELVATVTSHGTTNYADSVKGRFTISREGAKNTVYLQMNSLRPEDT |
| | | I51V, G54H, D72E, | AVYYCNAHFPGIYWGKGTLVTVSSAAAHHHHHH |
| | | N73G, L79Y, K83R, | |
| | | Q108L)-HIS6 | |
| 85 | P01500031 | 2HF09 Q1E, Q5V, | EVQLVESGGGLVQPGGSLRLSCAASESPFSGIIMGWYRQAPGKQR |
| | | A14P, G26E, G28P, | ELVATVTSHGTTNYADSVKGRFTISREGAKNTVYLQMNSLRPEDTA |
| | | M321, I51V, G54H, | VYYCNAHFPGIYWGKGTLVTVSSAAADYKDHDGDYKDHDIDYKDD |
| | | D72E, N73G, L79Y, | DDKGAAHHHHHHGGGGSRRPYIL |
| | | K83R, Q108L)- | |
| | | FLAG3-HIS6-4GS- | |
| | | NT°8-13) | |
| 86 | P01500032 | 2HF09 Q1E, Q5V, | EVQLVESGGGLVQPGGSLRLSCAASESPFSGPIMGWYRQAPGKQR |
| | | A14P, G26E, G28P, | ELVATVTSHGTTNYADSVKGRFTISREGAKNTVYLQMNSLRPEDTA |
| | | M32P, I51V, G54H, | VYYCNAHFPGIYWGKGTLVTVSSAAADYKDHDGDYKDHDIDYKDD |
| | | D72E, N73G, L79Y, | DDKGAAHHHHHHGGGGSRRPYIL |
| | | K83R, Q108L)- | |
| | | FLAG3-HIS6-4GS- | |
| | | NT°8-13) | |
| 87 | P01500070 | P01500001[E1D, | DVQLVESGGGLVQPGGSLRLSCAASGSGFSGMFMGWYRQAPGK |
| | | I33F, N73P]Flag3- | QRELVATVTSHGTTNYADSVKGRFTISRDPAKNTVYLQMNSLRPED |
| | | His6-GS-NT | |
| | | | TAVYYCNAHFPGIYWGKGTLVTVSSAAADYKDHDGDYKDHDIDYK |
| | | | DDDDKGAAHHHHHHGGGGSRRPYIL |
| 88 | P01500071 | P01500001[E1D, | DVQLVESGGGLVQPGGSLRLSCAASESGFSGMFMGWYRQAPGK |
| | | G26E, I33F] Flag3- | QRELVATVTSHGTTNYADSVKGRFTISRDNAKNTVYLQMNSLRPE |
| | | His6-GS-NT | DTAVYYCNAHFPGIYWGKGTLVTVSSAAADYKDHDGDYKDHDIDY |
| | | | KDDDDKGAAHHHHHHGGGGSRRPYIL |
| 89 | P01500072 | P01500001 [E1D, | DVQLVESGGGLVQPGGSLRLSCAASESPFSGMIMGWYRQAPGKQ |
| | | G26E, G28P, D72E, | RELVATVTSHGTTNYADSVKGRFTISREGAKNTVYLQMNSLRPEDT |
| | | N73G]Flag3-His6- | AVYYCNAHFPGIYWGKGTLVTVSSAAADYKDHDGDYKDHDIDYKD |
| | | GS-NT | DDDKGAAHHHHHHGGGGSRRPYIL |
| 90 | P01500073 | P01500001 [E1D, | DVQLVESGGGLVQPGGSLRLSCAASGSTFIGMIMGWYRQAPGKQ |
| | | G28T, S301]Flag3- | RELVATVTSHGTTNYADSVKGRFTISRDNAKNTVYLQMNSLRPEDT |
| | | His6-GS-NT | AVYYCNAHFPGIYWGKGTLVTVSSAAADYKDHDGDYKDHDIDYKD |
| | | | DDDKGAAHHHHHHGGGGSRRPYIL |
| 91 | P01500074 | P01500001 [E1D, | DVQLVESGGGLVQPGGSLRLSCAASESGFSGMLMGWYRQAPGK |
| | | G26E, I33L]Flag3- | QRELVATVTSHGTTNYADSVKGRFTISRDNAKNTVYLQMNSLRPE |
| | | His6-GS-NT | DTAVYYCNAHFPGIYWGKGTLVTVSSAAADYKDHDGDYKDHDIDY |
| | | | KDDDDKGAAHHHHHHGGGGSRRPYIL |
| 92 | P01500075 | P01500001 [E1D, | DVQLVESGGGLVQPGGSLRLSCAASGSGFSGMLMGWYRQAPGK |
| | | I33L, N73P]Flag3- | QRELVATVTSHGTTNYADSVKGRFTISRDPAKNTVYLQMNSLRPED |
| | | His6-GS-NT | TAVYYCNAHFPGIYWGKGTLVTVSSAAADYKDHDGDYKDHDIDYK |
| | | | DDDDKGAAHHHHHHGGGGSRRPYIL |
| 93 | P01500076 | P01500001 [E1D, | DVQLVESGGGLVQPGGSLRLSCAASESTFIGMLMGWYRQAPGKQ |
| | | G26E, G28T, S301, | RELVATVTSHGTTNYADSVKGRFTISRDNAKNTVYLQMNSLRPEDT |
| | | I33L]Flag3-His6- | AVYYCNAHFPGIYWGKGTLVTVSSAAADYKDHDGDYKDHDIDYKD |
| | | GS-NT | DDDKGAAHHHHHHGGGGSRRPYIL |
| 94 | P01500077 | P01500001 [E1D, | DVQLVESGGGLVQPGGSLRLSCAASGSTFIGMIMGWYRQAPGKQ |
| | | G28T, S301, N73P] | RELVATVTSHGTTNYADSVKGRFTISRDPAKNTVYLQMNSLRPEDT |
| | | Flag3-His6-GS-NT | AVYYCNAHFPGIYWGKGTLVTVSSAAADYKDHDGDYKDHDIDYKD |
| | | | DDDKGAAHHHHHHGGGGSRRPYIL |
| 95 | P01500079 | P01500001 [E1D] | DVQLVESGGGLVQPGGSLRLSCAASGSGFSGMIMGWYRQAPGK |
| | | Flag3-His6-GS-NT | QRELVATVTSHGTTNYADSVKGRFTISRDNAKNTVYLQMNSLRPE |
| | | | DTAVYYCNAHFPGIYWGKGTLVTVSSAAADYKDHDGDYKDHDIDY |
| | | | KDDDDKGAAHHHHHHGGGGSRRPYIL |

The binding of humanized variants of 2HFO42 was assessed by kinetic binding analysis to mouse and human FRα protein, biotinylated via an Avi-tag, captured on streptavidin biosensors by biolayer interferometry ^{∘}BLI), essentially as described earlier. VHHs were analysed at 100 nM concentration, and off-rates were determined using fitting of 1:1 langmurian interaction. Dissociation constants were compared to 2HFO42^{∘}Q1E, Q5V, Q108L) ^{∘}P101500004) and 2MFR67 ^{∘}Q1E, Q5V, Q108L) ^{∘}P0150000), respectively. Results are depicted in Table 8.

Secondly, the variants were analysed for the capacity to compete with binding of family 3 VHH 2MFR67 to human FRα in a competition AlphaLISA, which is a homogeneous assay without wash steps. In here, human FRα biotinylated through an Avi-tag ^{∘}FO1-H82E2, AcroBiosystems) is captured on streptavidin coated Alpha Donor beads ^{∘}Perkin Elmer, Cat nr. 6760002), while the 2MFR67 is captured on anti-Flag antibody AlphaLISA acceptor beads ^{∘}Perkin Elmer, Cat nr. AL112C). Binding of 2MFR67 to the FRα leads to an energy transfer from one bead to the other, ultimately producing a fluorescent signal. To each well of white low binding 384-well microtitre plates ^{∘}F-bottom, Greiner Cat nr 781904), 5 μl of serial-diluted VHHs are mixed with 5 μl of 2.5 nM monovalent 2MFR67-Flag3-His6 and 5 μl of 15 nM biotinylated human FRα protein. After an incubation for 1 hour at room temperature, streptavidin coated Alpha Donor beads and anti-Flag AlphaLISA acceptor beads were added to a final concentration of 20 μg/mL each in a final volume of 25 μl for an incubation of 1 hour at room temperature in the dark. Interaction between beads was assessed after illumination at 680 nm and reading at 615 nm of on an Ensight instrument. Curve fitting is done in Graphpad Prism 9.0 using 4PL non-linear regression analysis.

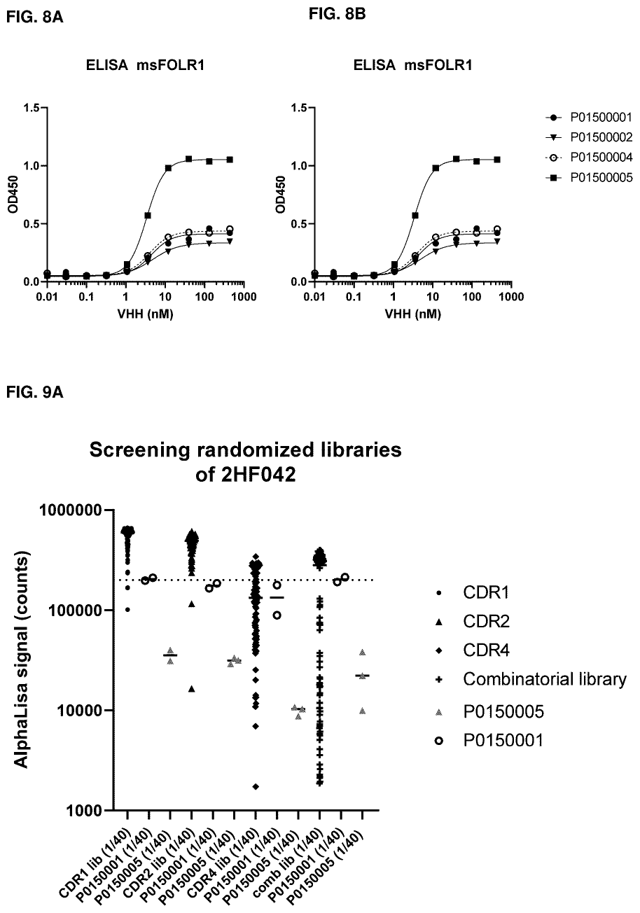

Dose-dependent competition of different FRα binding VHHs was assessed to determine the respective IC50 values ^{∘}Table 8). FIG. 7 shows dose-dependent inhibition of different VHHs competing for binding of MFR67 ^{∘}family 3) to hFRα in AlphaLISA. Results confirm that 2HFO19^{∘}Q1E, Q5V, Q108L) ^{∘}P01500003, fam 1) competes with 2MFR67 indicating that epitopes of these VHHS overlap. In addition, dose-dependent binding to mouse and cynomolgus FRα was assessed in ELISA ^{∘}FIG. 8A-B). The thermal stability of variants was analysed by testing the melting temperature in PBS at neutral pH and in acetate buffer at pH 5.5 ^{∘}Table 8).

TABLE 8
Overview in vitro characterization of humanized FRα binding variants
of 2HFO42 ^{°}fam3) variants, and 2HFO19 ^{°}fam1).

| | | | cAlphaLISA | | |
|---|---|---|---|---|---|
| | h FRα | m FRα | hFRα | Tm ^{°}°C.) | |
| | Koff ^{°}s−1) | Koff ^{°}s−1) | IC50 ^{°}M) | pH 7.4 | pH 5.5 |
| P01500001 | 1.10E−02 | 1.26E−03 | 3.73E−08 | 74.3 | 72.9 |
| P01500002 | 1.32E−02 | 3.07E−03 | 4.37E−08 | 73.0 | 71.5 |
| P01500003 | 4.99E−03 | 5.20E−04 | 7.55E−09 | | |
| P01500004 | 1.02E−02 | 1.93E−03 | 3.90E−08 | 70.6 | 68.5 |
| P01500005 | 3.53E−03 | 5.32E−04 | 5.53E−09 | 67.5 | 66.8 |
| P01500006 | 1.20E−02 | 2.52E−03 | | | |
| P01500007 | 1.64E−02 | 2.92E−03 | | | |
| P01500008 | 3.04E−03 | 4.20E−04 | | | |

### Example 10. Affinity Optimization of 2HFO42 Variants Towards Human FRα

Further, affinity optimization to human FOLR1 was done using a library approach by substituting amino acids in the CDR1, CDR2 and the positions forming the extra loop in framework 3 aka ^{∘}CDR4′ loop of the humanized variant of 2HFO42 ^{∘}P01500001)). Indeed, the FR3 region of heavy chain antibodies corresponds in its 3D structure to a 4^{th} loop region located on the same side of the Ig fold, the DE-loop, and is therefore considered as a potential ‘CDR4’ region, potentially involved in, or affecting, the antibody/antigen interaction. The FR3 region forming the DE loop contains positions 71-78 according to Kabat numbering ^{∘}Kelow et al., 2020; MABS 12/1, e1840005), and as previously observed, at least the 72-74 residue substitutions had an impact on affinity and/or crossing. So for affinity optimization efforts on 2HFO42 the CDR3 was not altered to avoid issues with binding specificity, but the CDR1, CDR2 and so-called CDR4 region were modified as to analyze the importance of these sequence regions of 2HFO42 for the functionality of this VHH in complex with FRα.

In a first round, the amino acids of the different CDR regions were substituted to all 20 amino acids, i.e. in separate single site saturation libraries. For the AffMatCDR1 library, nine residues were substituted ^{∘}G26-I33, G35), for AffMatCDR2 also nine residues ^{∘}T50-N58), for AffMatCDR4 6 positions ^{∘}D72-T77). After screening, the mutations of interest were combined in a new combinatorial library, that was again screened for improved binding towards human FOLR1.

For the generation of single site saturation libraries, mutations were introduced by site-directed mutagenesis PCR. For the single site saturation libraries, the codon of interest is mutated by three different primers containing the degenerative codons NDT, VHG and TGG in a 12:9:1 molar ratio ^{∘}22-ctrick method). This combination of primers codes for all 20 amino acids without any stop codon and only one redundant set for valine ^{∘}GTT and GTG) and leucine ^{∘}CTT and CTG). Individual PCR reactions for each position using h1 2HFO42 ^{∘}P01500001) as template were set-up. PCR products were purified using a gel extraction kit, and equimolar amounts of the purified PCR product were pooled per CDR region. In each library pool, the parental plasmid DNA was removed by performing a DpnI digestion for 60 min at 37° C., and subsequently purified using a PCR purification kit ^{∘}Qiagen). The newly synthesized DNA was ligated using T4 ligase and transformed into electrocompetent E. coli TOP10, generating a library size that covers at least 5× the theoretical diversity. For each AffMat library, as quality control 90 colonies were sequenced to confirm the library diversity.

Single colonies were picked and subsequently grown in 96-wells plates. To have a good coverage of the different variants, 4 times excess of the library diversity was picked. For each of the AffMatCDR1 and AffMatCDR2 libraries, 9 96-well plates were generated with each 90 clones per plate to allow the screening of single site variants. For AFFMATCDR4 library 5 96-wells plates were generated. As in-plate controls, the parental P01500001 and the family member 2MFR67 P01500005 were included in the same plate. Variants were produced from glycerol stocks in 96-Deep well plates containing 1 ml of 2×TY/Kanamycin overnight at 37° C., 250 rpm, after which periplasmic extracts were prepared from the bacterial cell pellets.

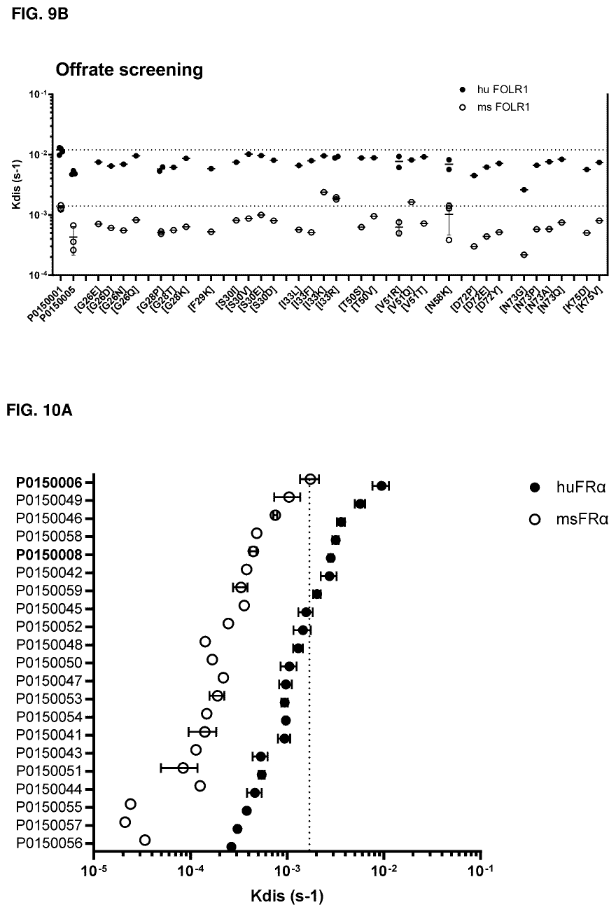

Variants were screened in competition AlphaLISA ^{∘}FIG. 9). The clones with an improved affinity to human FOLR1 compared to the in-plate control P01500001 were subsequently selected for sequence analysis, to deconvolute the mutation. Hits were sequenced, and subsequently single clones were analysed for off rates to human and mouse FRα in BLI. A selection of variants with >1.5-fold improved off-rates on human FOLR1 was also analysed for binding to human FOLR2 in BLI, to verify if the introduced mutation resulted in binding to human FOLR2. None of the point mutants showed detectable binding to human FOLR2.

Selected mutations identified to improve the off-rate to human FRα were combined in one combinatorial library. For the combinatorial library, dedicated mutations at 8 different positions were introduced in different rounds of site-directed mutagenesis. In CDR4, the mutations were pair-wise introduced in 6 combinations to prevent the introduction of unwanted potential post-translational modification sites. As a first step, the combinations of D72-N73, D72-P73, E72-G73, P72-G73, and P72-N73 were introduced in P01500001 in 6 separate PCR reactions using specific primers. Subsequently, the CDR1 and CDR2 mutations are added in each of these 6 reactions, resulting in a theoretical library size of 576. Similar as done for the single site CDR libraries, single colonies were picked for the generation of in total 12 96-wells plates of the AffMatCombo library for screening purposes. One plate was sequenced to assess the library quality.

Screening of the AffMatCombo library was done using competition AlphaLISA, in a dilution of 1:80 of periplasmatic extracts. FIG. 9A shows a representative example of the screening results in competition AlphaLISA of the combinatorial library, in comparison to the CDR1, CDR2, and CDR4 libraries.

The clones with an improved affinity to human FOLR1 compared to the in-plate controls P0150001- and P0150005 were sequenced to deconvolute the combinatorial mutations. A selection of 100 clones was subjected to off-rate analysis for binding to human FOLR1, mouse FOLR1 in BLI, using biotinylated FOLR1 proteins captured on streptavidin biosensors.

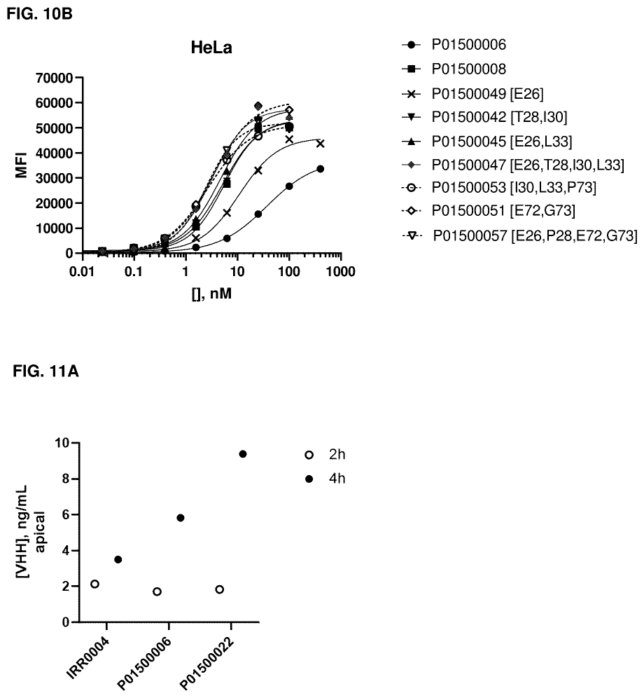

Selected affinity optimised variants with different combinations of up to 4 substitutions in the CDR1, CDR2 and CDR4 regions with a range of affinities to human FRα were produced as purified flag3-His6 tagged VHHs for further characterization ^{∘}FIG. 10, Table 9). Nanobodies were produced in TG-1 E. coli at 0.5 L scale, and purified from the periplasmatic fraction using standard affinity chromatography on 2 mL Ni Sepharose FastFlow columns, followed by desalting. Purity was determined by SIDS-PAGE.

Off-rates of purified variants were determined to human, mouse, and cynomolgus FRα in BLI. In addition, binding of the affinity optimised variants to cell-expressed human FRα on HeLa cells was determined by flow cytometry ^{∘}FIG. 10 B).

Results indicate that substitutions in the so called CDR4 region, in particular D72E-N73G and D72P-N73G substitutions gave the strongest improvements in off-rate to human FRs compared to the basic variant ^{∘}P01500006).

TABLE 9
Dissociation rates of affinity optimized variants of humanized 2HF042
to human and mouse FRα ^{°}biotinylated via avi-tag) by BLI.

| | hFRα | mFRα | | | | | | | | | |
|---|---|---|---|---|---|---|---|---|---|---|---|
| ID | Kdis (1/s) | Kdis (1/s) | Mutations | # | 26 | 28 | 30 | 33 | 51 | 72 | 73 |
| P0150006 | 9.67E−03 | 1.89E−03 | | | . | . | . | . | . | . | . |
| P0150019 | 2.06E−04 | 8.33E−05 | G26E, G28P, | 4 | E | P | . | . | . | E | G |
| | | | D72E, N73G | | | | | | | | |
| P0150056 | 2.95E−04 | 3.38E−05 | I33L, D72P, | 3 | . | . | . | L | . | P | G |
| | | | N73G | | | | | | | | |
| P0150057 | 3.16E−04 | 2.11E−05 | G26E, G28P, | 4 | E | P | . | . | . | E | G |
| | | | D72E, N73G | | | | | | | | |
| P0150055 | 3.82E−04 | 2.40E−05 | V51R, D72E, | 3 | . | . | . | . | R | E | G |
| | | | N73G | | | | | | | | |
| P0150051 | 4.74E−04 | 4.99E−05 | D72E, N73G | 2 | | | | | | E | G |
| P0150051 | 5.55E−04 | 8.33E−05 | D72E, N73G | 2 | . | . | . | . | . | E | G |
| P0150044 | 5.55E−04 | 1.26E−04 | S30I, D72E, | 3 | . | . | I | . | . | E | G |
| | | | N73G | | | | | | | | |
| P0150043 | 6.39E−04 | 1.14E−04 | G28T, I33L, | 3 | . | T | . | L | . | E | |
| | | | D72E | | | | | | | | |
| P0150053 | 9.22E−04 | 1.67E−04 | S30I, I33L, | 3 | . | . | I | L | . | . | P |
| | | | N73P | | | | | | | | |
| P0150054 | 9.37E−04 | 1.47E−04 | I33L, N73P | 2 | . | . | . | L | . | . | P |
| P0150053 | 1.05E−03 | 2.14E−04 | S30I, I33L, | 3 | . | . | I | L | . | . | P |
| | | | N73P | | | | | | | | |
| P0150041 | 1.13E−03 | 1.91E−04 | D72P, N73G | 2 | . | . | . | . | . | P | G |
| P0150047 | 1.19E−03 | 2.19E−04 | G26E, G28T, | 4 | E | T | I | L | . | . | |
| | | | S30I, I33L | | | | | | | | |
| P0150050 | 1.28E−03 | 1.68E−04 | G28T, D72P | 2 | . | T | . | . | . | P | . |
| P0150048 | 1.47E−03 | 1.42E−04 | S30I, D72P | 2 | . | . | I | . | . | P | . |
| P0150052 | 1.80E−03 | 2.46E−04 | V51R, D72E | 2 | . | . | . | . | R | E | . |
| P0150045 | 1.87E−03 | 3.60E−04 | G26E, I33L | 2 | E | . | . | L | . | . | . |
| P0150059 | 1.90E−03 | 2.92E−04 | G26E, D72E | 2 | E | . | . | . | . | E | . |
| P0150058 | 2.85E−03 | 4.84E−04 | G26E, G28P | 2 | E | P | . | . | . | . | . |
| P0150042 | 3.38E−03 | 3.80E−04 | G28T, S30I | 2 | . | T | I | . | . | . | . |
| P0150046 | 3.71E−03 | 7.77E−04 | N73P | 1 | . | . | . | . | . | . | P |
| P0150046 | 4.00E−03 | 7.31E−04 | N73P | 1 | . | . | . | . | . | . | P |
| P0150049 | 6.48E−03 | 8.26E−04 | G26E | 1 | E | . | . | . | . | . | . |

HeLa cells

| | | | | cyFO | | |
|---|---|---|---|---|---|---|
| | | BLI | | LR1 | | |
| | Substitutions in | huFOLR1 | mFOLR1 | Kdis | | EC₅₀ |
| ID | P01500001 | K_{dis} (s − 1) | K_{dis} (s − 1) | (s − 1) | MFI top | (nM) |
| P01500056 | [I33L, D72P, | 2.65E−04 | 3.38E− | 3.40E− | 55336 | 2.52 |
| | N73G] | | 05 | 04 | | |
| P01500057 | [G26E, G28P, | 3.07E−04 | 2.11E− | 2.34E− | 53784 | 2.56 |
| | D72E, N73G] | | 05 | 04 | | |
| P01500055 | [V51R, D72E, | 3.82E−04 | 2.40E− | 4.24E− | 58975 | 3.77 |
| | N73G] | | 05 | 05 | | |
| P01500044 | [S30I, D72E, | 4.64E−04 | 1.26E− | 7.37E− | 64056 | 3.29 |
| | N73G] | | 04 | 04 | | |
| P01500051 | [D72E, N73G] | 5.46E−04 | 8.41E− | 7.80E− | 61005 | 3.31 |
| | | | 05 | 04 | | |
| P01500043 | [G28T, I33L, | 5.34E−04 | 1.14E− | 4.28E− | 60215 | 2.40 |
| | D72E] | | 04 | 04 | | |
| P01500041 | [D72P, N73G] | 9.40E−04 | 1.40E− | 1.40E− | 63050 | 4.20 |
| | | | 04 | 04 | | |
| P01500054 | [I33L, N73P] | 9.68E−04 | 1.47E− | 1.47E− | 55840 | 5.18 |
| | | | 04 | 04 | | |
| P01500053 | [S301, I33L, | 9.43E−04 | 1.91E− | 1.67E− | 51210 | 2.63 |
| | N73P] | | 04 | 04 | | |
| P01500047 | [G26E, G28T, | 9.71E−04 | 2.19E− | 2.19E− | 59061 | 3.19 |
| | S301, I33L] | | 04 | 04 | | |
| P01500050 | [G28T, D72P] | 1.05E−03 | 1.68E− | 1.09E− | 62536 | 3.61 |
| | | | 04 | 03 | | |
| P01500048 | [S301, D72P] | 1.30E−03 | 1.42E− | 1.33E− | 59849 | 2.46 |
| | | | 04 | 03 | | |
| P01500052 | [V51R, D72E] | 1.46E−03 | 2.46E− | 2.81E− | 53942 | 4.35 |
| | | | 04 | 03 | | |
| P01500045 | [G26E, I33L] | 1.57E−03 | 3.60E− | 3.60E− | 58907 | 4.71 |
| | | | 04 | 04 | | |
| P01500059 | [G26E, D72E] | 2.04E−03 | 3.33E− | 2.92E− | nd | nd |
| | | | 04 | 04 | | |
| P01500042 | [G28T, S30I] | 2.74E−03 | 3.80E− | 3.80E− | 54691 | 5.19 |
| | | | 04 | 04 | | |
| P01500008 | | 2.82E−03 | 4.47E− | 4.54E− | 54862 | 5.6 |
| | | | 04 | 04 | | |
| P01500058 | [G26E, G28P] | 3.18E−03 | 4.84E− | 3.69E− | nd | nd |
| | | | 04 | 03 | | |
| P01500046 | [N73P] | 3.61E−03 | 7.54E− | 3.60E− | 50778 | 6.91 |
| | | | 04 | 03 | | |
| P01500049 | [G26E] | 5.69E−03 | 1.05E− | 3.93E− | 46582 | 10.9 |
| | | | 03 | 04 | | |
| P01500006 | | 9.47E−03 | 1.74E− | 1.58E− | 35302 | 32.4 |
| | | | 03 | 03 | | |

### Example 11. In Vitro Transcytosis Assay Using Human HIPCPP Cells

To assess the capacity of FRα binding VHHs to cross the BCSFB in a human setting, an in vitro transcytosis assay with immortalized HIBCPP cells was done, modified from Dinner et al. ^{∘}2016 J Vis Exp e54061). As controls for transcytosis served an anti-Tfr1 Nb188 ^{∘}De Wilde et al. 2020), and an irrelevant control VHH ^{∘}IRR4). All samples were analysed in replicates.

For VHH uptake and transport measurements, 1×10⁵ HIBCPP cells were seeded in transwell culture inserts ^{∘}growth area 0.3 cm², pore size 0.4 mm, pore density 4×10⁶ pores/cm², polyester membrane). Cells are trypsinized with trypsin 0.25% for 20 min at 37° C., washed hereafter and seeded onto filters ^{∘}0.1 ml, at a seeding density of 1×10⁵ cells/insert). For the standard cell culture, 0.5 ml of medium is added to 24-well plate and inserts are placed in each well. For the inverted cell culture, cells are plated in previously described inserts that are flipped over and placed in a medium flooded 12-well plate. Cells are incubated for 24 h at 37° C. in 5% CO₂. At the following day, cells cultured in standard set-up are fed by adding 0.1 ml of fresh medium to each insert. For the inverted set-up, culture inserts are flipped over again and transferred to 24-well plate ^{∘}containing 1 ml of cell culture medium) and 0.2 ml of fresh medium is added to each insert. HIBCPP are cultured in DMEM/F12+GlutaMax ^{∘}Gibco, 31331) supplemented with 10% FBS and 5 μg/mL of insulin during 3 days subsequently it is replaced to folate-free RPMI medium ^{∘}Gibco, 27016) supplemented with 1% FBS and 5 μg/mL of insulin. Upon confluency and when cells reach TEER values of 500 Ω·cm², transcytosis was assessed by addition of 100 nM VHH in culture medium to the basolateral compartment, and culturing for 4 hours at 37° C. in 5% CO₂. Samples from basolateral and apical side are recovery and stored at −20° C. until further analysis.

To quantify the VHHs in the cell culture medium from apical side, a sandwich ELISA approach was used. In 96-Well Nunc-Immuno™ ^{∘}MaxiSorp) plates were coated with 80 ng of AffiniPure Goat Anti-Alpaca IgG, VHH domain, polyclonal antibody ^{∘}Jackson ImmunoResearch, cat n. 128-005-232) at 4° C., O/N. Samples were incubated for 1 h at RT. Detection was performed by using 100 ng of anti-His Antibody [HRP], mAb, mouse ^{∘}GenScript, A00612), 30 min. at RT. Interpolation was done to a standard curve using 4PL analysis in GraphPad Prism 9.0.

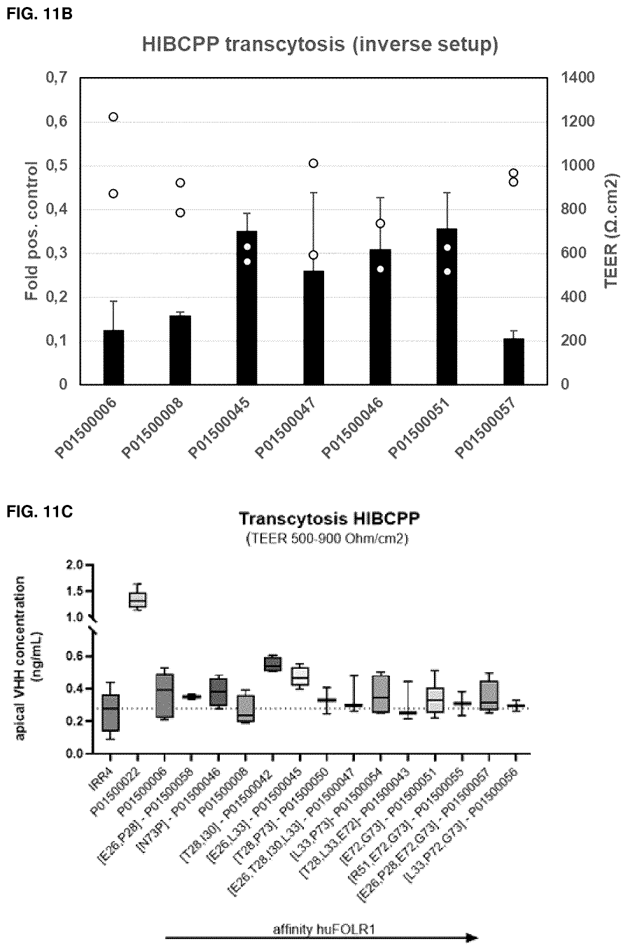

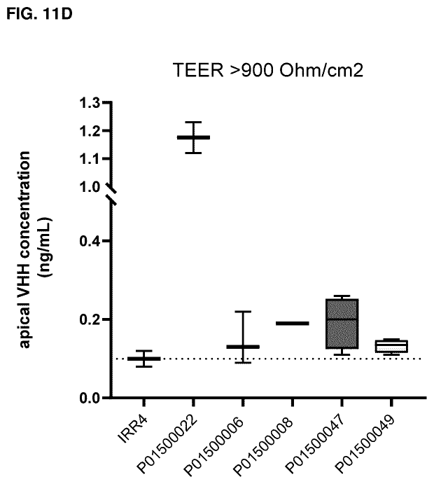

The panel of affinity optimised variants of 2HFO42 was analysed in the HIBCPP transcytosis assay. In addition to the anti-hTfr Nb188 ^{∘}P01500022) used as positive control, P01500042, P01500045, P01500047 and P01500006, were found to be able to get transported to the apical compartment ^{∘}FIG. 11).

### Example 12. Co-Crystal Structures of FRα and FRα Binding VHHs

Co-crystallization studies were performed on human FRα to determine the exact epitope of the FRα binding agents herein disclosed. Recombinant human FRα ^{∘}29-234) protein was produced in special low-glycosylation HEK293 cell lines. Proteins are fully deglycosylated using EndoH. Nanobodies were produced in TG-1 E. coli at 2 L scale, and purified from the periplasmatic fraction using standard affinity chromatography on 2 mL Ni Sepharose FastFlow columns. After analysis on SDS-PAGE, the pool was injected on a Superdex 75 size exclusion column equilibrated in 50 mM MES pH 6.0, 150 mM NaCl, and concentrated to 10 mg/mL.

Co-crystallization structures of hFRα with 2HFO42 ^{∘}Q1E, Q5V, Q108L) ^{∘}P01500004), and 2HFO19 ^{∘}Q1E, Q5V, Q108L)_P01500003), and 2MFR67 ^{∘}Q1E, Q5V, Q108L) ^{∘}P01500005) were initiated. Nanobodies were added to FOLR1 protein in a 1.2 times molar excess. Crystallisation was performed using the sitting drop vapour diffusion technique, using the Mosquito crystallisation robot to set up crystallisation drops containing 0.1 ul protein sample+0.1 ul bottom solution. Crystallisation screening was performed using commercially available screening kits from Molecular Dimensions. X-ray data were collected at 100 K at the Soleil and Diamond synchrotron facilities. X-ray data were processed either using the xdsme processing pipeline ^{∘}Legrand, 2017) or the autoPROC+Staraniso processing pipeline ^{∘}Vonrhein et al., 2011, Vonrhein et al., 2018).

The structures of the 2HFO19-FOLR1 and 2HFO42-FOLR1 complexes were solved using molecular replacement using PDB entries 4lrh and 7s0e as models for FOLR1 and 2HF)19, respectively, using the Phaser program ^{∘}McCoy et al., 2007) from the Phenix suite ^{∘}Adams et al., 2010). The structure was further manually build in Coot ^{∘}Emsley and Cowtan, 2004) and refined using phenix.refine ^{∘}Afonine et al., 2012) from the Phenix suite. The structure of the 2MFR67-FOLR1 complex was subsequently solved using molecular replacement, using the 2HFO42-FOLR1 structure, and also further build manually in Coot and refined using phenix.refine.

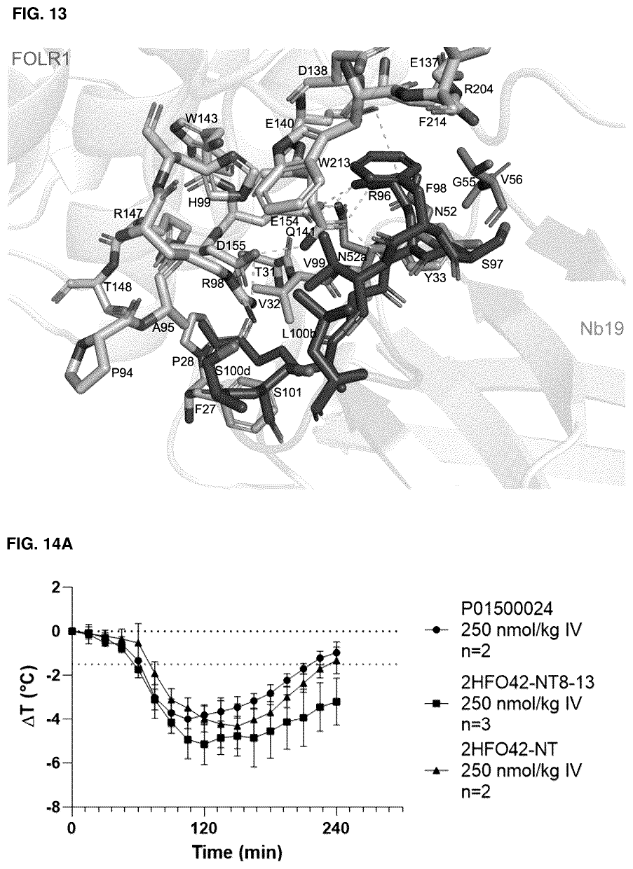

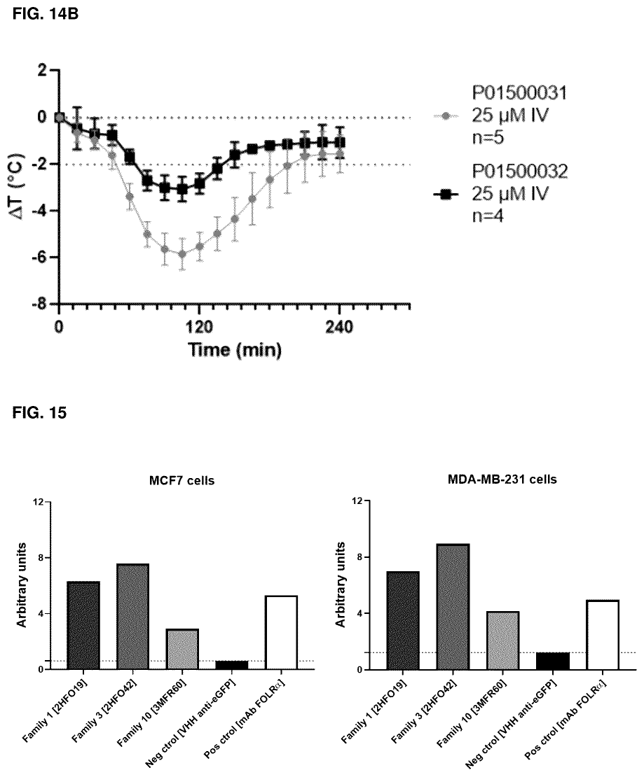

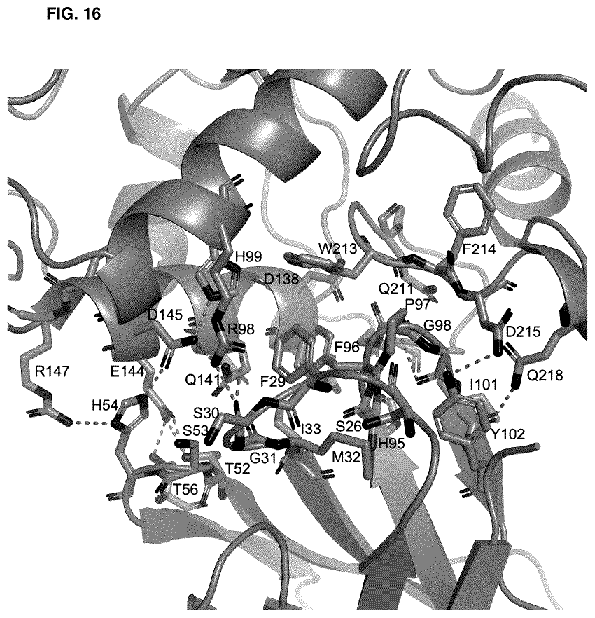

2HFO19 ^{∘}P01500003)-FOLR1 was crystallized at neutral pH ^{∘}6.5) at 2.8 Å resolution. These experiments revealed that 2HFO19 binds a conformational epitope on hFRα comprising the amino acid residues P94, A95, R98, H99, E137, D138, E140, Q141, W143, E144, D145, R147, T148, R204, W213 and F214 ^{∘}FIG. 13). The paratope of 2HFO19 comprises amino acid residues with the CDR1, CDR2 and CDR3 regions, more particularly F27, P28, T31, V32 and Y33 ^{∘}from CDR1), N52, N53, G56 and V57 ^{∘}from CDR2) and R98, R99, R100, S101, F102, V103, L106, S108 and S109 ^{∘}from CDR3).

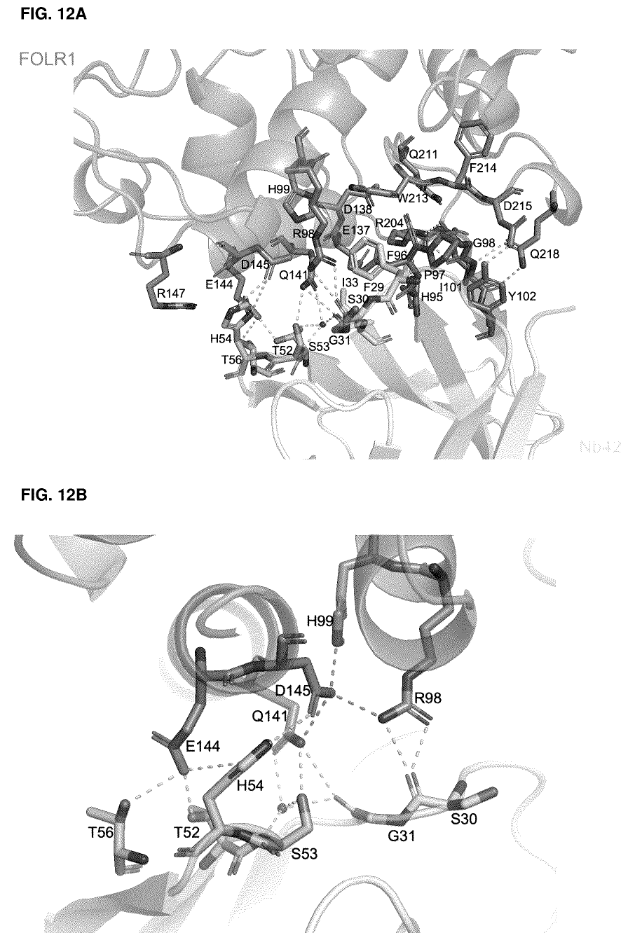

2HFO42 ^{∘}P01500004)-FOLR1 was crystallized at neutral pH ^{∘}6.5) at 2.23 Å resolution. The AU contains 2 copies of FOLR1 and 2 copies of Nb42, forming 2 Nb42-FOLR1 complexes. The BCSFB crossing 2HFO42 binds a conformational epitope on hFRα comprising the amino acid residues R98, H99, E137, D138, Q141, E144, D145, R204, G205, Q211, W213, F214, D215, A217 and Q218 ^{∘}FIG. 12). The paratope of 2HFO42 comprises the amino residues F29, S30, G31 and I33 ^{∘}from CDR1), T52, S53, H54 and T56 ^{∘}from CDR2), and H95, F96, P97, G98, I101, Y102^{∘} from CDR3, according to Kabat numbering).

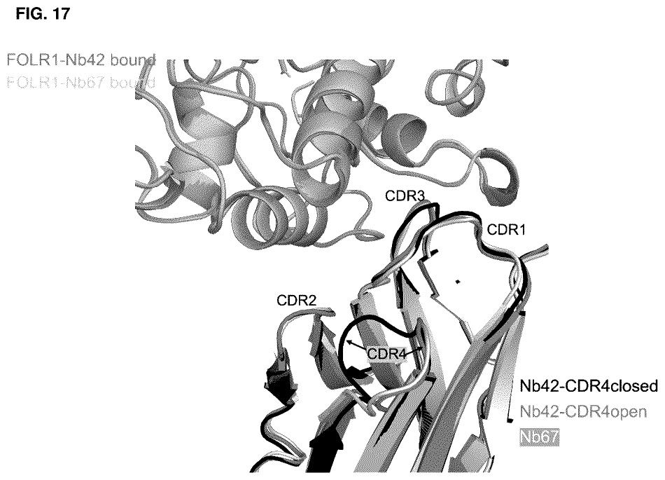

In the two copies of 2HFO42 a different conformation of the CDR4 region located within framework 3 is observed, indicating flexibility of this loop. The CDR4 is either pointed towards the CDR2 region in a “closed” conformation, or the CDR4 is directed away in an “open” conformation, in which K75 forms H-bonds to S30. The conformation of the CDR4 does not affect the conformation of CDR1 or CDR2. In both conformations, H-bonds are formed between the CDR1 and CDR2 backbones and the side chains of R71 and N76 in CDR4. In the closed conformation, an extra H-bond is formed between the backbone of A74 in the CDR4 and the backbone of 553 in CDR2. While the conformation of CDR4 does not affect the interface with FOLR1, it result in a different surface and the “open” conformation leaves a positive charged cavity between CDR1-CDR2 and CDR4 ^{∘}FIG. 17).

To address the relevance of the CDR4 amino acids and orientation for functionality, the co-crystal structure of human FOLR1 extracellular domain with 2MFR67 was determined at 3.09 Å resolution at neutral pH 6.5. The sequence of 2MFR67 differs from 2HFO42 at position 40 and 72, 73, and 74 in the CDR4 loop within framework 3. The interface with human FOLR1 is similar for both 2HFO42 and 2MFR67 family 3 members: H-bonds are formed between 2MFR67 S30, T52, H54, T56, H98, P97, G98, I101 and Y102 ^{∘}according to Kabat numbering), and FOLR1 R98, Q141, E144, D145, R147, R204, D215 and Q218. The structure shows that in 2MFR67 the CDR4 region is exclusively found in the open conformation, directed away from the CDR regions, and also in this case it does not make an interaction with FOLR1.

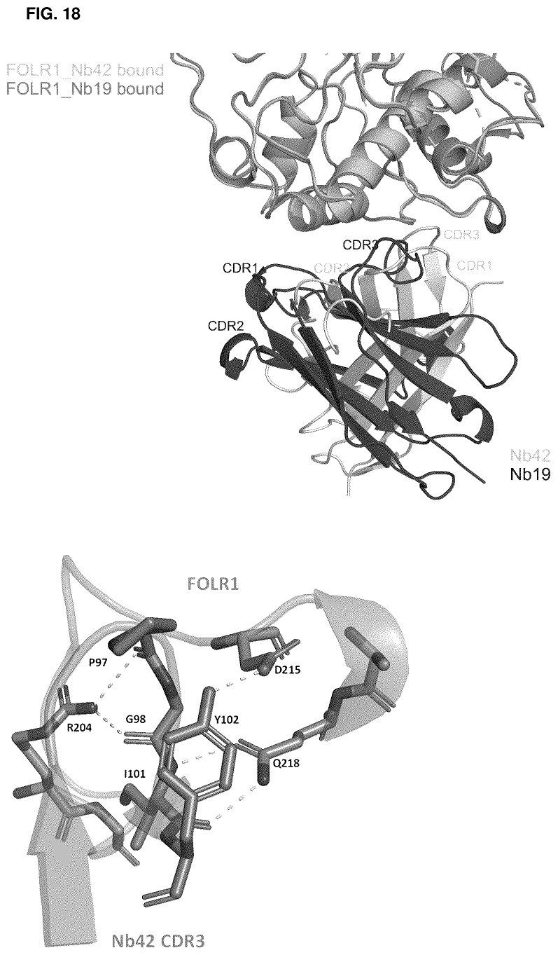

2MFR67 and 2HFO42 bind FOLR1 in the same mode, different from the binding mode of 2HFO19 Nanobody family 1. While they all bind to the “core epitope” surrounding human FOLR1 Q141, the paratopes are different, and 2HFO42 and 2MFR67 pick up extra interactions with FOLR1 residues 204 and 213-218 through their CDR3 region ^{∘}FIG. 18).

### Example 13. Binding to FRα Expressing Breast Cancer Derived Cell Lines

It has been demonstrated that cancers of epithelial origin, such as cancers of the ovary, breast, pleura, lung, cervix, endometrium, colon, kidney, bladder and brain overexpress FOLR1. Given that the VHHs herein described bind to a new FRα epitope, we assessed whether the FRα binding VHHs demonstrate improved binding characteristics towards FOLR1 expressing cancer cells, more particularly to FOLR1-positive breast cancer derived cell lines. This was evaluated by dot blot in 0.5 μg of human MCF7 and MDA-MB-231 cell lysates. Human FOLR1-expressing MCF7 and MDA-MB-231 breast tumor derived cells were homogenized in 0.5% CHAPS/PBS at 20 Hz during 3 min with Tissue Lyser II and afterwards centrifuged at 13.000 rpm 4° C. during 5 min. Cell lysate (500 ng) or lysis buffer ((−) control) alone were spotted in a nitrocellulose membrane, dried during 15 min and blocked during 1 hour at room temperature with 3% BSA in TBS 0.1% Tween (TBS-T). Subsequently, membranes were incubated ON at 4° C. with 75 μg/ml of VHH or 0.75 μg/ml monoclonal antibody in 1.5% BSA in TBST. After 3 washes with TBST, membranes were incubated with an anti-His 1:5000 antibody during 1 h at room temperature. Membranes were washed 3× with TBST and incubated next with a secondary antibody conjugated with Alexa Fluor 680 during 1 h at room temperature. After 3 washes with TBST, the blots were scanned and quantified with ImageJ software. A human FRα binding monoclonal antibody (mAb) was used as positive control. The loss of binding of the monoclonal antibody to FRα chimera 6 (as explained in Example 5) demonstrates that the mAb binds a different epitope. Surprisingly, as can be seen in FIG. 15, the family 1 and family 3 FRα binding VHHs recognizing the novel FRα epitope showed improved binding to MCF7 and MDA-MD-231 cells compared to the positive control.

### Example 14: Isothermal Titration Colorimetry Studies

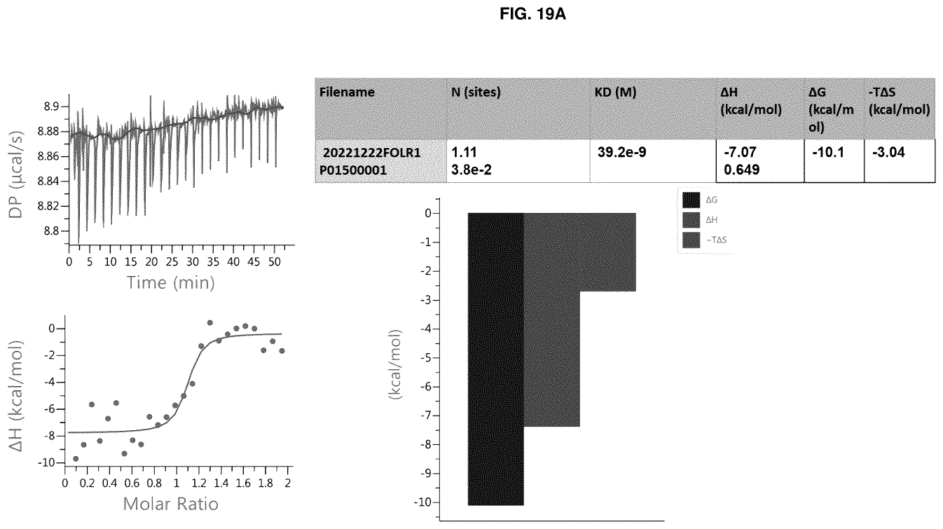

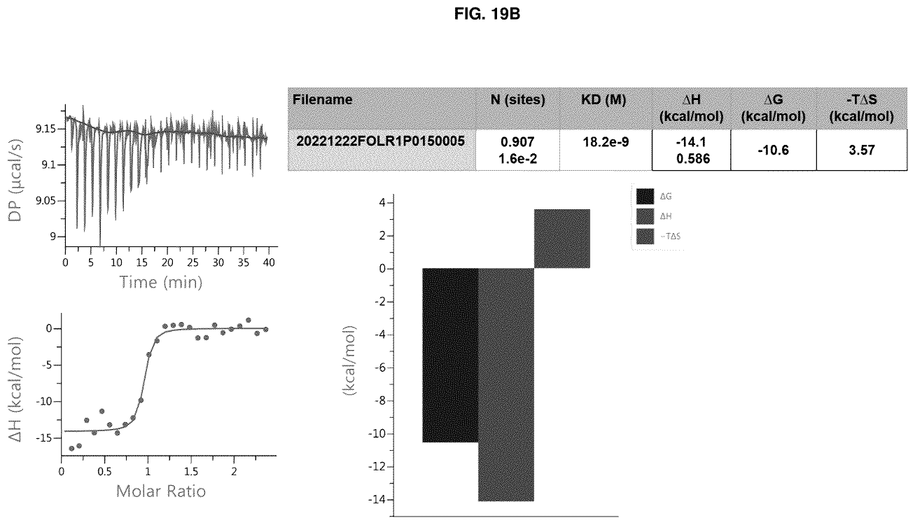

The structure indicates that the higher affinity of 2MFR67 towards human FOLR1 is not mediated by direct interaction of CDR4 residues to the receptor, but suggests that the residues have impact on the conformation of the CDR4 region. To exclude the presence of a potential additional binding site mediated by the CDR4 region, we determined the stoichiometry if the humanized variant h1 2HFO42 (P01500001) and h1 2MFR67 (P01500005) interaction with human FOLR1 in isothermal titration colorimetry (ITC) studies, using glycosylated human FOLR protein (AcroBiosystems Cat. Nr F01-H52H1). All proteins were dialysed overnight to PBS buffer and concentrated using Amicon Ultra 10 kDa cut-off centrifugal filter devices. Human FOLR1 was used at a concentration of 6.5 μM in the cell. Titrations comprised 26×1.5 μL injections of VHH into the protein, with 90 s intervals. An initial injection of ligand (0.4 μL) was made and discarded during data analysis. All data were performed at room temperature. The data were fitted to a single binding site model using the PEAQ ITC analysis software provided by the manufacturer. Results are shown in FIGS. 19A and B.

ITC measurements show that the stoichiometry of the 2HFO42-FOLR1 interaction is 1:1, indicating that there is no 2^{nd} interaction occurring in solution. Similarly, a stoichiometry of 1:1 was determined for the 2MFR67-FOLR1 interaction. When comparing the thermodynamic parameters, binding of 2MFR67 to FOLR1 comes with an entropic penalty, whereas the entropic contribution for humanised 2HFO42-FOLR1 binding is favorable. This could be a consequence of the CDR4 loop having more freedom in 2MFR67, resulting in an entropic penalty upon binding. On the other hand, this degree of freedom also allows the paratope to find a better fit, which in turn results in a higher enthalpic contribution. In conclusion, these data confirm that the CDR4 region within 2MFR67 is not involved in direct receptor interaction, and that there is a 1:1 stoichiometry.

### Example 15: Biophysical Characterization of Affinity Optimized 2HFO42 Variants

Protein stability during the transcytosis process with acidification during trafficking is important for functionality. To assess the impact of the introduced substitutions on the stability of the affinity optimised variants, we determined the temperature-induced unfolding (Tₘ) and aggregation (T_{agg}). Intrinsic tryptophan-fluorescence was monitored upon temperature-induced protein unfolding in the UNcle instrument (Unchained Labs; Pleasanton, CA, USA). Briefly, 10 μL of variants at 0.5 mg/mL in PBS (pH 7.4), and 10 mM acetate buffer (pH 5.5) was applied to the sample cuvette, and a linear temperature ramp was initiated from 25 to 95° C. at a rate of 0.1° C./min, with a pre-run incubation for 180 s. The barycentric mean (BCM) and static light scattering (SLS at 266 nm and 473 nm) signals were plotted against temperature in order to obtain melting temperatures (Tm) and aggregation onset temperatures (T_{agg}), respectively.

Results are shown in Table 10. In conclusion, in the majority of variants the introduced substitutions are not greatly affecting the thermal stability, which exception of the V51R mutation which has a negative impact. The N73P and combined G28T-S301 substitutions result in the highest Tm values, irrespective of the type of tags.

TABLE 10
Melting temperatures (Tm) of affinity optimized 2HFO42 variants
and control VHHs. Tm was determined at neutral and acidic
conditions, respectively, on UNcle instrument.

| | | | Sequence | | |
|---|---|---|---|---|---|
| | | | annotation: | | |
| | | | Substitutions over | Tm | Tm |
| | | | P01500001 in [ ]; or | (° C.) | (° C.) |
| | | | name of different | pH | pH |
| ID | tag | Host | VHH sequence | 7.4 | 5.5 |
| P01500001 | His6 | E. coli | | 74.3 | 72.9 |
| P01500005 | His6 | E. coli | 2MFR67 | 67.5 | 66.8 |
| P01500019 | His6 | E. coli | [G26E, G28P, | 70.7 | 70.8 |
| | | | D72E, N73G] | | |
| P01500041 | Flag3-His6 | E. coli | [P72, N73G] | 71.7 | 71 |
| P01500042 | Flag3-His6 | E. coli | [G28T, S30I] | 76.7 | 76.1 |
| P01500043 | Flag3-His6 | E. coli | [G28T, I33L, | 71.8 | 71.3 |
| | | | D72E] | | |
| P01500044 | Flag3-His6 | E. coli | [S30I, D72E, | 71.8 | 72.3 |
| | | | N73G] | | |
| P01500045 | Flag3-His6 | E. coli | [G26E, I33L] | 71 | 71.4 |
| P01500046 | Flag3-His6 | E. coli | [N73P] | 76.3 | 76.7 |
| P01500047 | Flag3-His6 | E. coli | [G26E, G28T, | 71.2 | 71.2 |
| | | | S30I, I33L] | | |
| P01500048 | Flag3-His6 | E. coli | [S30I, D72P] | 70.4 | 71.2 |
| P01500049 | Flag3-His6 | E. coli | [G26E] | 72.8 | 73.7 |
| P01500050 | Flag3-His6 | E. coli | [G28T, D72P] | 72.1 | 71.7 |
| P01500051 | Flag3-His6 | E. coli | [D72E, N73G] | 73.1 | 72.5 |
| P01500052 | Flag3-His6 | E. coli | [V51R, D72E] | 63.3 | 62.3 |
| P01500053 | Flag3-His6 | E. coli | [S30I, I33L, | 75.3 | 75.5 |
| | | | N73P] | | |
| P01500054 | Flag3-His6 | E. coli | [I33L, N73P] | 74.9 | 76.2 |
| P01500055 | Flag3-His6 | E. coli | [V51R, D72E, | 62.9 | 62.4 |
| | | | N73G] | | |
| P01500056 | Flag3-His6 | E. coli | [I33L, D72P, | 69.1 | 69.5 |
| | | | N73G] | | |
| P01500057 | Flag3-His6 | E. coli | [G26E, G28P, | 70.0 | 70.7 |
| | | | D72E, N73G] | | |
| P01500058 | Flag3-His6 | E. coli | [G26E, G28P] | 73.9 | 73.8 |
| P01500059 | Flag3-His6 | E. coli | [G26E, D72E] | 69.9 | 70.7 |
| P01500070 | Flag3-His6- | HEK-F | [E1D, I33F, | 71.4 | 72.3 |
| | GS-NT | | N73P] | | |
| P01500071 | Flag3-His6- | HEK-F | [E1D, G26E, | 67.4 | 68.9 |
| | GS-NT | | I33F] | | |
| P01500072 | Flag3-His6- | HEK-F | [E1D, G26E, | 69.1 | 68.8 |
| | GS-NT | | G28P, D72E, | | |
| | | | N73G] | | |
| P01500073 | Flag3-His6- | HEK-F | [E1D, G28T, | 75.2 | 75.4 |
| | GS-NT | | S30I] | | |
| P01500074 | Flag3-His6- | HEK-F | [E1D, G26E, | 70.7 | 71.2 |
| | GS-NT | | I33L] | | |
| P01500075 | Flag3-His6- | HEK-F | [E1D, I33L, | 73.8 | 74.6 |
| | GS-NT | | N73P] | | |
| P01500076 | Flag3-His6- | HEK-F | [E1D, G26E, | 70.9 | 70.8 |
| | GS-NT | | G28T, S30I, | | |
| | | | I33L] | | |
| P01500077 | Flag3-His6- | HEK-F | [E1D, G28T, | 76.0 | 75.8 |
| | GS-NT | | S30I, N73P] | | |
| P01500079 | Flag3-His6- | HEK-F | [E1D] | 76.2 | 75.9 |
| | GS-NT | | | | |

### Example 16. In Vivo Crossing of Affinity Optimized Variants in Transgenic Human FOLR1 Mice Model

Selected affinity optimised variants of 2HFO42 were generated as Neurotensin fusions, to allow the testing of the respective BCSFB crossing capacity in transgenic mice containing the human FOLR1 knock-in mice. Variants with a broad range in off-rates to human FOLR1 were selected, based on the results of the in vitro transcytosis assay in HIBCPP cells, binding affinity, and the stability assessment. In particular, we included AffMat variants with substitutions in CDR1 only, as well as variants that contained mutations in both CDR1 and CDR4 regions. Variants were cloned into a pcDNA3.4 expression vector containing C-terminal Flag3-His6-GS-NT(8-13) tags for expression in HEK-F cells at 300 mL scale, to obtain endotoxin-free proteins P01500070-79. During the recloning an additional E1D mutation was introduced, to avoid pyroglutamate formation. Purification was done as described previously herein. All purified variants P01500070-79 were analysed on SDS-PAGE and MS analysis to confirm purity and correct mass. As quality control, P01500070-79 were analysed for binding to human and mouse FOLR1 in off-rate analysis in BLI, and thermal stability (Table 11).

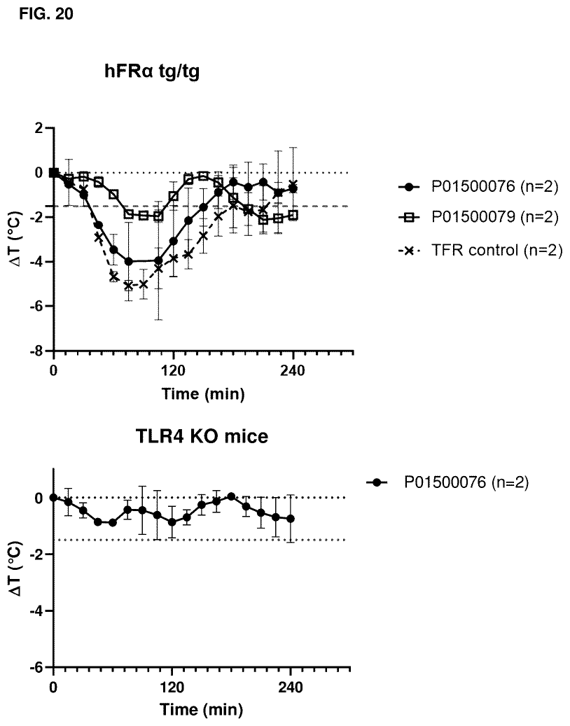

The generation of transgenic human FOLR1 mice was done using the CRISPR/Cas9 method. Since our VHHs are cross-reactive to mouse and human FOLR1, it was essential that the murine FRα is first knocked out and replaced by the human FRα, as to obtain the transgenic mice. Homozygeous hFOLR1 tg/tg mice were used for the Neurotensin hypothermia model, and implanted with probes, as described in example 6. FIG. 20 shows the results of body temperature changes after iv injection of different variants at 250 nmol/kg dose. The anti-mTFR VHH-NT served as system control, and showed hypothermia. The humanised 2HFO42, P01500079, was not inducing hypothermia in hFRα tg/tg mice, indicating that the affinity towards human FRα was not optimal. In contrast, the affinity optimised variant P01500076 containing 4 substitutions in the CDR1 [G26E, G28T, S301, 133L] induced hypothermia, indicating its off-rate to hFRα (1.12E-03 1/s) was optimal for BCSFB crossing in this humanised system. In the mouse system, no crossing was observed with P01500076, due to a too high affinity to mFRα.

TABLE 11
Binding analysis to human and mouse FOLR1 of affinity
optimized variants of 2HFO42 as Neurotensin-fusions
produced in Hek-F cells for in vivo studies (BLI).

| | Substitutions in | Ms FOLR1 | HuFOLR1 |
|---|---|---|---|
| ID | P01500001 | kdis(1/s) | kdis (1/s) |
| P01500072 | [E1D, G26E, G28P, D72E, | 2.1E−05 | 3.07E−04 |
| | N73G] | | |
| P01500076 | [E1D, G26E, G28T, S30I, | 1.64E−04 | 1.12E−03 |
| | I33L] | | |
| P01500075 | [E1D, I33L, N73P] | 1.25E−04 | 1.25E−03 |
| P01500070 | [E1D, I33F, N73P] | 1.92E−04 | 1.51E−03 |
| P01500074 | [E1D, G26E, I33L] | 3.28E−04 | 1.81E−03 |
| P01500077 | [E1D, G28T, S30I, N73P] | 3.35E−04 | 2.17E−03 |
| P01500071 | [E1D, G26E, I33F] | 3.26E−04 | 2.66E−03 |
| P01500073 | [E1D, G28T, S30I] | 4.97E−04 | 3.46E−03 |
| P01500079 | [E1D] | 2.24E−03 | 1.01E−02 |

### Example 17. Effect of Size on Crossing of 2HFO42

To analyse whether the potential use as shuttle to the CSF, we analysed the impact of size on the BCSFB crossing capacity of 2HFO42. To this end, 2HFO42 (P01500004) was engineered into a bivalent format by genetic fusion. In here, 2HFO42 was fused to a flexible [GGGGS]3 linker, followed by either an irrelevant VHH, anti-eGFP VHH, or a second 2HFO42 VHHs, and introduced in an expression vector containing a C-terminal Flag3-His6-GS-NT₈₋₁₃ peptide.

The NT-fusion proteins were produced in E. coli, and purified from the periplasmatic fraction using standard affinity chromatography on 2 mL Ni Sepharose FastFlow columns The purity and the integrity of the purified proteins were verified by SDS-Page and MS-analysis, respectively. For quality control, the binding to mouse FOLR1 protein was measured using BLI.

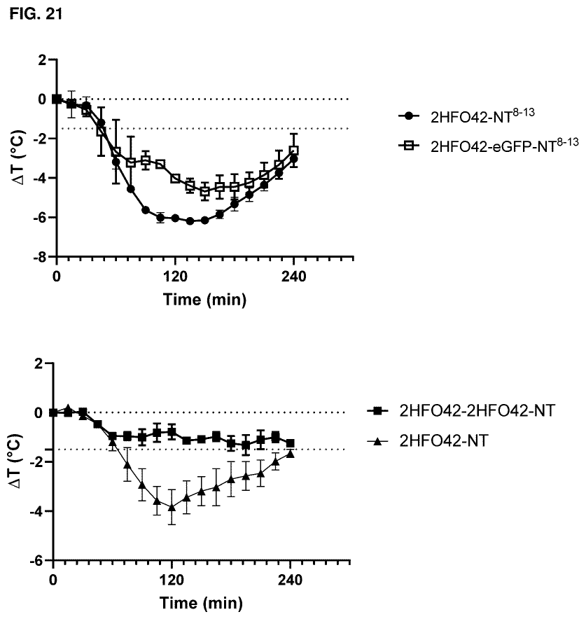

To assess functional crossing to brain, the bivalent 2HFO42 NT-fusions were analysed for the induction of hypothermia in the Neurotensin mouse model described in Example 6. Each construct was injected IV at a dose of 250 nmol/kg (n=4). As reference monovalent 2HFO42-NT was used. Results are shown in FIG. 21. These results indicate that a bivalent format of 2HFO42 fused to a non-FOLR1 VHH (anti-eGFP VHH) as cargo can cross the BCSFB, but that a bivalent 2HFO42 VHH is not showing a functional response in the NT model, likely due to such a high binding affinity of the bivalent 2HFO42 Nb to the receptor that release is not efficient.

### Example 18. Removal of Post-Translational Methionine Oxidation Site in 2HFO42

Within the CDR1 region of 2HFO42, the conserved methionine at position 32 is prone to oxidation in forced oxidation experiments, and substitution is desired for manufacturability. To this end single site substitution libraries were generated in which M32 was substituted to any other amino acid except for cysteine in the humanized variant of 2HFO42 (P0150001). All variants were subjected to off-rate analysis to mouse FOLR1 and human FOLR1 using BLI, as described before. Subsequently variants (Table 13) with the most beneficial substitutions, M32P, M32I and M32L were purified from large scale E. coli productions for further characterization (P01500012, P01500013, and P01500014, respectively). In addition, the M32I/P mutations were introduced in two VHHs that have different CDR4 residues and higher affinity to FOLRα, 2MFR67 (resulting in P01500016 and P01500017) and an affinity optimized variant P01500019 with [G26E, G28P, D72E, N73G] substitutions (resulting in P01500015 and P01500016, respectively).

Purified Nanobodies were characterized for binding to human and mouse FOLRα in BLI, to cynomolgus FOLRα in ELISA, and for stability by Tm analysis using Uncle. Binding results showed that in all backgrounds, the substitution of M32 to proline or isoleucine comes with a loss in binding to FolRα, with reduced binding levels and with faster dissociation rates. The penalty on binding appears larger for 2MFR67 and the affinity improved variant P01500019, which indicate that the local context of the CDR4 region is important. Stability assessment of the panel indicated that the M32P mutation gives a strong reduction in Tm compared to the wildtype variant, while the M32I mutation is benign and in case of P01500019 even increases the Tm value.

To overcome the negative impact of M32 mutation on stability and FOLRα binding affinity, we introduced additional compensatory mutations predicted by the algorithm FoldX based on the structure of 2HF42-hFOLR1 complex to compensate for either the M32P or M32I substitution (Schymkowitz et al. 2005; Nucleic Acids Res.; 33, Issue suppl_2, W382-W388). Variants listed in Table 12 have been generated as Flag3-His6 tagged proteins for characterization in binding assays and Tm determination, for which results are also indicated.

TABLE 12
Removal of Met32 PTM site in humanized 2HFO42.

| | | | BLI mFOLR1 | | BLI hFOLR1 | | |
|---|---|---|---|---|---|---|---|
| | | | | nm | | nm | Tm |
| | Mutations in | SEQ | | (Fold | | (Fold | pH |
| ID | P0150001 | ID | kdis(1/s) | wt) | kdis(1/s) | wt) | 7.4° C. |
| P01500006 | | 42 | 1.62E−03 | 1 | 1.32E−02 | 1 | 74.3 |
| P01500014 | M32P | 98 | 2.41E−03 | 1.5 | 2.55E−02 | 2 | 70.7 |
| P01500086 | A24L, S27A, M32P | 104 | 3.50E−03 | 2.1 | 2.40E−02 | 1.8 | 74.2 |
| P01500087 | A24L, S27A, M32P, N67E | 105 | 3.76E−03 | 2.3 | 2.69E−02 | 2 | 70.4 |
| P01500085 | A24V, S27L, M32P | 103 | 1.76E−03 | 1.1 | 1.59E−02 | 1.2 | 74.5 |
| P01500090 | A24V, S27L, M32P, N67E | 108 | 3.46E−03 | 2.1 | 2.59E−02 | 2 | 71 |
| P01500012 | M32I | 99 | 7.62E−03 | 4.7 | 7.12E−02 | 5.4 | 74.5 |
| P01500082 | A24V, S27L, M32I | 100 | 6.18E−02 | 38 | >> | > | 77.8 |
| P01500083 | A24V, S27L, M32I, N76D | 101 | 9.75E−02 | 60 | >> | > | 73.5 |
| P01500084 | A24V, S27T, M32I, N76D | 102 | 6.49E−03 | 4 | 3.36E−02 | 2.5 | 71.7 |
| P01500088 | S27T, M32I | 106 | 1.54E−02 | 9.4 | 3.81E−01 | 29 | 74.9 |
| P01500089 | S27T, M32I, N76D | 107 | 6.14E−03 | 3.8 | 7.47E−02 | 5.7 | 71.5 |

TABLE 13
Sequences of further optimized VHH constructs.

| SEQ ID No. | Name | Amino acid sequence |
|---|---|---|
| 98 | P01500014 | EVQLVESGGGLVQPGGSLRLSCAASGSGFSGPIMGWYRQAPGKQRELVATVTS |
| | | HGTTNYADSVKGRFTISRDNAKNTVYLQMNSLRPEDTAVYYCNAHFPGIYWGK |
| | | GTLVTVSSAAAHHHHHH |
| 99 | P01500012 | EVQLVESGGGLVQPGGSLRLSCAASGSGFSGIIMGWYRQAPGKQRELVATVTSH |
| | | GTTNYADSVKGRFTISRDNAKNTVYLQMNSLRPEDTAVYYCNAHFPGIYWGKGT |
| | | LVTVSSAAAHHHHHH |
| 100 | P01500082 | EVQLVESGGGLVQPGGSLRLSCAVSGLGFSGIIMGWYRQAPGKQRELVATVTSH |
| | | GTTNYADSVKGRFTISRDNAKNTVYLQMNSLRPEDTAVYYCNAHFPGIYWGKGT |
| | | LVTVSSAAADYKDHDGDYKDHDIDYKDDDDKGAAHHHHHH |
| 101 | P01500083 | EVQLVESGGGLVQPGGSLRLSCAVSGLGFSGIIMGWYRQAPGKQRELVATVTSH |
| | | GTTNYADSVKGRFTISRDNAKDTVYLQMNSLRPEDTAVYYCNAHFPGIYWGKGT |
| | | LVTVSSAAADYKDHDGDYKDHDIDYKDDDDKGAAHHHHHH |
| 102 | P01500084 | EVQLVESGGGLVQPGGSLRLSCAVSGTGFSGIIMGWYRQAPGKQRELVATVTSH |
| | | GTTNYADSVKGRFTISRDNAKDTVYLQMNSLRPEDTAVYYCNAHFPGIYWGKGT |
| | | LVTVSSAAADYKDHDGDYKDHDIDYKDDDDKGAAHHHHHH |
| 103 | P01500085 | EVQLVESGGGLVQPGGSLRLSCAVSGLGFSGPIMGWYRQAPGKQRELVATVTS |
| | | HGTTNYADSVKGRFTISRDNAKNTVYLQMNSLRPEDTAVYYCNAHFPGIYWGK |
| | | GTLVTVSSAAADYKDHDGDYKDHDIDYKDDDDKGAAHHHHHH |
| 104 | P01500086 | EVQLVESGGGLVQPGGSLRLSCALSGAGFSGPIMGWYRQAPGKQRELVATVTS |
| | | HGTTNYADSVKGRFTISRDNAKNTVYLQMNSLRPEDTAVYYCNAHFPGIYWGK |
| | | GTLVTVSSAAADYKDHDGDYKDHDIDYKDDDDKGAAHHHHHH |
| 105 | P01500087 | EVQLVESGGGLVQPGGSLRLSCALSGAGFSGPIMGWYRQAPGKQRELVATVTS |
| | | HGTTNYADSVKGRFTISRDNAKETVYLQMNSLRPEDTAVYYCNAHFPGIYWGKG |
| | | TLVTVSSAAADYKDHDGDYKDHDIDYKDDDDKGAAHHHHHH |
| 106 | P01500088 | EVQLVESGGGLVQPGGSLRLSCAASGTGFSGIIMGWYRQAPGKQRELVATVTSH |
| | | GTTNYADSVKGRFTISRDNAKNTVYLQMNSLRPEDTAVYYCNAHFPGIYWGKGT |
| | | LVTVSSAAADYKDHDGDYKDHDIDYKDDDDKGAAHHHHHH |
| 107 | P01500089 | EVQLVESGGGLVQPGGSLRLSCAASGTGFSGIIMGWYRQAPGKQRELVATVTSH |
| | | GTTNYADSVKGRFTISRDNAKDTVYLQMNSLRPEDTAVYYCNAHFPGIYWGKGT |
| | | LVTVSSAAADYKDHDGDYKDHDIDYKDDDDKGAAHHHHHH |
| 108 | P01500090 | EVQLVESGGGLVQPGGSLRLSCAVSGLGFSGPIMGWYRQAPGKQRELVATVTS |
| | | HGTTNYADSVKGRFTISRDNAKETVYLQMNSLRPEDTAVYYCNAHFPGIYWGKG |
| | | TLVTVSSAAADYKDHDGDYKDHDIDYKDDDDKGAAHHHHHH |
| 109 | P01500013 | EVQLVESGGGLVQPGGSLRLSCAASGSGFSGLIMGWYRQAPGKQRELVATVTS |
| | | HGTTNYADSVKGRFTISRDNAKNTVYLQMNSLRPEDTAVYYCNAHFPGIYWGK |
| | | GTLVTVSSAAAHHHHHH |
| 110 | P01500015 | EVQLVESGGGLVQPGGSLRLSCAASESPFSGIIMGWYRQAPGKQRELVATVTSH |
| | | GTTNYADSVKGRFTISREGAKNTVYLQMNSLRPEDTAVYYCNAHFPGIYWGKGT |
| | | LVTVSSAAAHHHHHH |
| 111 | P01500016 | EVQLVESGGGLVQPGGSLRLSCAASESPFSGPIMGWYRQAPGKQRELVATVTSH |
| | | GTTNYADSVKGRFTISREGAKNTVYLQMNSLRPEDTAVYYCNAHFPGIYWGKGT |
| | | LVTVSSAAAHHHHHH |
| 112 | P01500017 | EVQLVESGGGLVQAGGSLRLSCAASGSGFSGIIMGWYRQAPGKQRELVATVTSH |
| | | GTTNYADSVKGRFTISREDSKNTVLLQMNSLKPEDTAVYYCNAHFPGIYWGKGT |
| | | LVTVSSAAAHHHHHH |

1. A folate receptor alpha (FRα) binding agent comprising a means for specifically binding human FRα with a dissociation constant k_{off} of less than 3×10⁻²/s as determined by biolayer interferometry,
wherein the FRα binding agent specifically binds to the human FRα epitope comprising amino acid Q141 of SEQ ID NO: 1, and
wherein the FRα binding agent specifically binds human FRα at an epitope of at least two or more amino acids selected from R98, H99, E137, D138, 0141, E144, D145, R204, G205, Q211, W213, F214, D215, A217 and/or Q218 of SEQ ID NO: 1.

2. (canceled)

3. The FRα binding agent of claim 1, comprising an immunoglobulin single variable domain (ISVD) specifically binding human FRα.

4. The FRα binding agent of claim 3, wherein the ISVD binds the human FRα via the paratope comprising amino acid residue positions 29, 30, 31 and 33 in CDR1, and 52, 53, 54 and 56 in CDR2, and 95, 96, 97, 98, 101, and 102 in CDR3 according to Kabat numbering for VHH 2HFO42 presented in SEQ ID No. 2.

5. The FRα binding agent of claim 3, wherein the ISVD comprises amino acids D72 and N73, or E72 and G73, or P72 and G73, in FR3 according to Kabat numbering.

6. The FRα binding agent of claim 4, wherein the ISVD comprises amino acids R71, A74, K75, N76, and T77 in FR3, and D72 and N73, or E72 and G73, or P72 and G73, in FR3 according to Kabat numbering.

7. The FRα binding agent of claim 4, wherein the ISVD comprises a CDR3 sequence as presented in SEQ ID No. 5 or consists of an amino acid sequence with maximally two amino acids different from SEQ ID No. 5.

8. The FRα binding agent of claim 3, wherein the k_{off} is between 3×10⁻² and 1×10⁻³/s.

9. The FRα binding agent of claim 3, wherein the binding to human FRα does not interfere with folate binding and/or folate transport by the human FRα.

10. The FRα binding agent of claim 3, wherein the binding agent is capable of cross reacting with primate and mouse FRα.

11. The FRα binding agent of claim 3, wherein said binding agent comprises an ISVD comprising a CDR3 sequence according to SEQ ID No.5, a CDR2 sequence as depicted in SEQ ID No. 31 and/or SEQ ID No. 32, and/or SEQ ID No.4, and a CDR1 sequence as depicted in SEQ ID No.113, or SEQ ID No. 3.

12. The FRα binding agent of claim 3, wherein said ISVD comprises a CDR1, CDR2, CDR3 sequence as present in SEQ ID No.2, wherein the CDRs are annotated according to Chothia, AbM, MacCallum, IMGT or Kabat.

13. The FRα binding agent of claim 11, wherein the ISVD comprises a FR1 sequence according to SEQ ID No.114, a FR2 sequence according to SEQ ID No.115, a FR3 sequence according to SEQ ID No.116 and a FR4 sequence according to SEQ ID No.117, or a sequence with at least 90% identity over the full length of said sequence, wherein the CDR1, 2, 3 and 4 regions are identical.

14. The FRα binding agent of claim 3, wherein the ISVD comprises or consists of amino acid sequence SEQ ID No. 2, or SEQ ID No. 118-121, or a homologue with at least 90% identity thereof over the full length of said sequence, wherein the CDR1, 2, 3 and 4 regions are identical to any one of SEQ ID No. 2, or SEQ ID No.118-121, or a humanized variant thereof.

15. The FRα binding agent of claim 1, wherein said FRα binding agent when coupled to a chemical entity facilitates the uptake of the chemical entity into the cerebrospinal fluid (CSF) across the blood CSF barrier (BCSFB) or into FRα expressing cancer cells or enables the binding of the chemical entity to FRα expressing cancer cells.

16. The FRα binding agent of 15, wherein said chemical entity is a biological, small molecule, therapeutic agent, a radionuclide, an antisense oligonucleotide, imaging agent or test compound.

17. The FRα binding agent of claim 15, wherein said chemical entity is neurotensin or a neurotensin analogue.

18. The FRα binding agent of claim 1, wherein the binding agent comprises or consists of an antibody or an antibody fragment, and/or which is a multispecific binding agent.

19.-35. (canceled)

36. A nucleic acid molecule encoding the FRα binding agent of claim 1.

37. A vector comprising the nucleic acid molecule according to claim 36.

38. A host cell comprising the nucleic acid molecule according to claim 36.
